(19) United States
(12) Patent Application Publication
(10) Pub. No.: US 2014/0080732 A1
(43) Pub. Date: Mar. 20, 2014

(54) BLOOD TRANSCRIPTIONAL SIGNATURE OF ACTIVE VERSUS LATENT MYCOBACTERIUM TUBERCULOSIS INFECTION

(71) Applicants: Baylor Research Institute, Dallas, TX (US); Imperial College Healthcare NHS Trust, London (GB); Medical Research Council, Swindon (GB)

(72) Inventors: Jacques F. Banchereau, Montclair, NJ (US); Damien Chaussabel, Bainbridge Island, WA (US); Anne O'Garra, London (GB); Matthew Berry, London (GB); Onn Min Kon, London (GB)

(73) Assignees: Baylor Research Institute, Dallas, TX (US); Imperial College Healthcare NHS Trust, London (GB); Medical Research Council, Swindon (GB)

(21) Appl. No.: 14/024,142

(22) Filed: Sep. 11, 2013

Related U.S. Application Data
(63) Continuation of application No. 12/628,148, filed on Nov. 30, 2009, now abandoned.
Application No. 12/628,148 is a continuation-in-part of application No. 12/602,488, filed on Apr. 25, 2011.
(60) Provisional application No. 61/075,728, filed on Jun. 25, 2008.

(51) Int. Cl. C12Q 1/68

(52) U.S. Cl. CPC C12Q 1/689
USPC 506/9

(57) ABSTRACT

The present invention includes methods, systems and kits for distinguishing between active and latent mycobacterium tuberculosis infection in a patient suspected of being infected with Mycobacterium tuberculosis, the method including the steps of obtaining a patient gene expression dataset from a patient suspected of being infected with Mycobacterium tuberculosis; sorting the patient gene expression dataset into one or more gene modules associated with Mycobacterium tuberculosis infection; and comparing the patient gene expression dataset for each of the one or more gene modules to a gene expression dataset from a non-patient; wherein an increase or decrease in the totality of gene expression in the patient gene expression dataset for the one or more gene modules is indicative of active Mycobacterium tuberculosis infection.

### CROSS-REFERENCE TO RELATED APPLICATIONS

This application is a continuation of U.S. patent application Ser. No. 12/628,148, filed Nov. 30, 2009, which is a continuation-in-part of U.S. patent application Ser. No. 12/602,488, which is a 35 U.S.C. 371 National Phase filing of PCT Application No. PCT/US09/048,698 filed Jun. 25, 2009, which claims priority to U.S. Provisional Application Ser. No. 61/075,728, filed Jun. 25, 2008. The entire contents of these disclosures are incorporated herein by reference.

### STATEMENT OF FEDERALLY FUNDED RESEARCH

This invention was made with U.S. Government support under National Institutes of Health Contract Nos. R01-01 AR46589, CA78846 and U19 A1057234-02. The government has certain rights in this invention.

### TECHNICAL FIELD OF THE INVENTION

The present invention relates in general to the field of Mycobacterium tuberculosis infection, and more particularly, to a method, kit and system for the diagnosis, prognosis and monitoring of active Mycobacterium tuberculosis infection and disease progression before, during and after treatment that appears latent or asymptomatic.

### BACKGROUND OF THE INVENTION

Without limiting the scope of the invention, its background is described in connection with the identification and treatment of Mycobacterium tuberculosis infection.

Pulmonary tuberculosis (PTB) is a major and increasing cause of morbidity and mortality worldwide caused by Mycobacterium tuberculosis (M. tuberculosis). However, the majority of individuals infected with M. tuberculosis remain asymptomatic, retaining the infection in a latent form and it is thought that this latent state is maintained by an active immune response (WHO; Kaufmann, SH & McMichael, AJ., Nat Med, 2005). This is supported by reports showing that treatment of patients with Crohn's Disease or Rheumatoid Arthritis with anti-TNF antibodies, results in improvement of autoimmune symptoms, but on the other hand causes reactivation of TB in patients previously in contact with M. tuberculosis (Keane). The immune response to M. tuberculosis is multifactorial and includes genetically determined host factors, such as TNF, and IFN-γ and IL-12, of the Th1 axis (Reviewed in Casanova, Ann Rev; Newport). However, immune cells from adult pulmonary TB patients can produce IFN-γ, IL-12 and TNF, and IFN-γ therapy does not help to ameliorate disease (Reviewed in Reljic, 2007, J Interferon & Cyt Res., 27, 353-63), suggesting that a broader number of host immune factors are involved in protection against M. tuberculosis and the maintenance of latency. Thus, knowledge of host factors induced in latent versus active TB may provide information with respect to the immune response, which can control infection with M. tuberculosis.

The diagnosis of PTB can be difficult and problematic for a number of reasons. Firstly demonstrating the presence of typical M. tuberculosis bacilli in the sputum by microscopy examination (smear positive) has a sensitivity of only 50-70%, and positive diagnosis requires isolation of M. tuberculosis by culture, which can take up to 8 weeks. In addition, some patients are smear negative on sputum or are unable to produce sputum, and thus additional sampling is required by bronchoscopy, an invasive procedure. Due to these limitations in the diagnosis of PTB, smear negative patients are sometimes tested for tuberculin (PPD) skin reactivity (Mantoux). However, tuberculin (PPD) skin reactivity cannot distinguish between BCG vaccination, latent or active TB. In response to this problem, assays have been developed demonstrating immunoreactivity to specific M. tuberculosis antigens, which are absent in BCG. Reactivity to these M. tuberculosis antigens, as measured by production of IFN-γ by blood cells in Interferon Gamma Release Assays (IGRA), however, does not differentiate latent from active disease. Latent TB is defined in the clinic by a delayed type hypersensitivity reaction when the patient is intradermally challenged with PPD, together with an IGRA positive result, in the absence of clinical symptoms or signs, or radiology suggestive of active disease. The reactivation of latent/dormant tuberculosis (TB) presents a major health hazard with the risk of transmission to other individuals, and thus biomarkers reflecting differences in latent and active TB patients would be of use in disease management, particularly since anti-mycobacterial drug treatment is arduous and can result in serious side-effects.

The majority of individuals infected with M. tuberculosis remain asymptomatic, with a third of the world's population estimated to be latently infected with the bacteria, thus providing an enormous reservoir for spread of disease. Of these persons described as latently infected, 5-15% will develop active TB disease in their lifetime^{7,8}. Thus, latent TB patients represent a clinically heterogeneous classification, ranging from the majority who will remain asymptomatic throughout their lives, to those who will progress to disease reactivation. The diagnosis of latent TB is based solely on evidence of immune sensitization, classically by the skin reaction to M. tuberculosis antigens, a test whose specificity is compromised by positive reactions to non-pathogenic mycobacteria including the vaccine BCG. More recent assays that determine the secretion of IFN-γ by blood cells to specific M. tuberculosis antigens (IGRA) suffer this problem less but, like the skin test, cannot differentiate latent from active disease, nor clearly identify those patients who may progress to active disease. Identification of those most at risk of reactivation would help with targeted preventative therapy, of importance since anti-mycobacterial drug treatment is lengthy and can result in serious side-effects. Thus new tools for diagnosis, treatment and vaccination are urgently needed, but efforts to develop these have been limited by an incomplete understanding of the complex underlying pathogenesis of TB.

### SUMMARY OF THE INVENTION

The present invention includes methods and kits for the identification of latent versus active tuberculosis (TB) patients, as compared to healthy controls. In one embodiment, microarray analysis of blood of a distinct and reciprocal immune signature is used to determine, diagnose, track and treat latent versus active tuberculosis (TB) patients. The present invention provides for the first time the ability to distinguish between the heterogeneity of TB infections can be used to determine which individuals with latent TB should be given anti-mycobacterial chemotherapy due to active and not latent/asymptomatic TB infection.

In one embodiment, the present invention includes a method for predicting an active Mycobacterium tuberculosis infection that appears latent/asymptomatic comprising: obtaining a patient gene expression dataset from a patient suspected of being infected with Mycobacterium tuberculosis; sorting the patient gene expression dataset into one or more gene modules associated with Mycobacterium tuberculosis infection; and comparing the patient gene expression dataset for each of the one or more gene modules to a gene expression dataset from a non-patient also sorted into the same gene modules; wherein an increase or decrease in the totality of gene expression in the patient gene expression dataset for the one or more gene modules is indicative of active Mycobacterium tuberculosis infection rather than a latent/asymptomatic Mycobacterium tuberculosis infection. In one aspect, the method further comprises the step of using the determined comparative gene product information to formulate at least one of diagnosis, a prognosis or a treatment plan. In another aspect, the method may also include the step of distinguishing patients with latent TB from active TB patients. In one aspect, the patient gene expression dataset is from cells in at least one of whole blood, peripheral blood mononuclear cells, or sputum. In another aspect, the patient gene expression dataset is compared to at least 10, 20, 40, 50, 70, 80, 90, 100, 125, 150, 200, 250, 300, 350 or 393 genes selected from the genes in Table 2. In another aspect, the patient gene expression dataset is compared to at least 10, 20, 40, 50, 70, 80, 90, 100, 125, 150, 200, Modules M1.3, M2.8, M1.5, M2.6, M2.2 and 3.1. In another aspect, the gene modules associated with Mycobacterium tuberculosis infection are selected from the group consisting of Module M1.3, Module M2.8, Modules M1.5, Modules M2.6, Module M2.2 and Module 3.1. In another aspect, the gene modules associated with Mycobacterium tuberculosis infection are selected with changes in a decrease in B cell-related genes, a decrease in T cell-related genes, an increase in myeloid related genes, an increase in neutrophil related transcripts and interferon inducible (IFN) genes. In another aspect, the patient's disease state is further determined by radiological analysis of the patient's lungs. In another aspect, the method also includes the step of determining a treated patient gene expression dataset after the patient has been treated and determining if the treated patient gene expression dataset has returned to a normal gene expression dataset thereby determining if the patient has been treated.

In another embodiment the present invention is a method for distinguishing between active and latent Mycobacterium tuberculosis infection in a patient suspected of being infected with Mycobacterium tuberculosis, the method comprising: obtaining a first gene expression dataset obtained from a first clinical group with active Mycobacterium tuberculosis infection, a second gene expression dataset obtained from a second clinical group with a latent Mycobacterium tuberculosis infection patient and a third gene expression dataset obtained from a clinical group of non-infected individuals; generating a gene cluster dataset comprising the differential expression of genes between any two of the first, second and third datasets; and determining a unique pattern of expression/representation that is indicative of latent infection, active infection or being healthy, wherein the patient gene expression dataset comprises at least 6, 10, 20, 40, 50, 70, 80, 90, 100, 125, 150, or 200 genes obtained from the genes in at least one of Modules M1.3, M2.8, M1.5, M2.6, M2.2 and 3.1.

In yet another embodiment the present invention is a kit for diagnosing infection in a patient suspected of being infected with Mycobacterium tuberculosis, the kit comprising: a gene expression detector for obtaining a patient gene expression dataset from the patient wherein the genes expressed are obtained from the patient's whole blood; and a processor capable of comparing the gene expression dataset to a pre-defined gene module dataset associated with Mycobacterium tuberculosis infection and that distinguish between infected and non-infected patients, wherein whole blood demonstrates an aggregate change in the levels of polynucleotides in the one or more transcriptional gene expression modules as compared to matched non-infected patients, thereby distinguishing between active and latent Mycobacterium tuberculosis infection. In one aspect, the patient gene expression dataset is obtained from peripheral blood mononuclear cells. In another aspect, the patient gene expression dataset is compared to at least 10, 20, 40, 50, 70, 80, 90, 100, 125, 150, 200, 250, 300, 350 or 393 genes selected from the genes in Table 2. In another aspect, the patient gene expression dataset is compared to at least 10, 20, 40, 50, 70, 80, 90, 100, 125, 150, 200, Modules M1.3, M2.8, M1.5, M2.6, M2.2 and 3.1. In another aspect, the gene modules associated with Mycobacterium tuberculosis infection are selected from the group consisting of Module M1.3, Module M2.8, Modules M1.5, Modules M2.6, Module M2.2 and Module 3.1. In another aspect, the gene modules associated with Mycobacterium tuberculosis infection are selected with changes in a decrease in B cell-related genes, a decrease in T cell-related genes, an increase in myeloid related genes, an increase in neutrophil related transcripts and interferon inducible (IFN) genes. In another aspect, the genes are selected from PDL-1, CASP5, CR1, CASP5, TLR5, MAPK14, STX11, BCL6 and C5.

Another embodiment of the present invention is a system of diagnosing a patient with active and latent Mycobacterium tuberculosis infection comprising: a gene expression detector for obtaining a patient gene expression dataset from the patient wherein the genes expressed are obtained from the patient's whole blood; and a processor capable of comparing the gene expression dataset to a pre-defined gene module dataset associated with Mycobacterium tuberculosis infection and that distinguish between infected and non-infected patients, wherein whole blood demonstrates an aggregate change in the levels of polynucleotides in the one or more transcriptional gene expression modules as compared to matched non-infected patients, thereby distinguishing between active and latent Mycobacterium tuberculosis infection, wherein the gene module dataset comprises at least one of Modules M1.3, M2.8, M1.5, M2.6, M2.2 and 3.1. In one aspect, the patient gene expression dataset is compared to at least 10, 20, 40, 50, 70, 80, 90, 100, 125, 150, 200, 250, 300, 350 or 393 genes selected from the genes in Table 2. In another aspect, the patient gene expression dataset is compared to at least 10, 20, 40, 50, 70, 80, 90, 100, 125, 150, 200, Modules M1.3, M2.8, M1.5, M2.6, M2.2 and 3.1. In another aspect, the gene modules associated with Mycobacterium tuberculosis infection are selected from the group consisting of Module M1.3, Module M2.8, Modules M1.5, Modules M2.6, Module M2.2 and Module 3.1. In another aspect, the gene modules associated with Mycobacterium tuberculosis infection are selected with changes in a decrease in B cell-related genes, a decrease in T cell-related genes, an increase in myeloid related genes, an increase in neutrophil related transcripts and interferon inducible (IFN) genes. In another aspect, the genes are selected from PDL-1, CASP5, CR1, CASP5, TLR5, MAPK14, STX11, BCL6 and C5.

### BRIEF DESCRIPTION OF THE DRAWINGS

For a more complete understanding of the features and advantages of the present invention, reference is now made to the detailed description of the invention along with the accompanying figures and in which:

FIGS. 1a to 1c. A distinct whole blood transcriptional signature of active TB. Each row of the heatmap represents an individual gene and each column an individual participant. The relative abundance of transcripts throughout the paper is indicated by a colour scale at the base of the figure (red, high; yellow, median; blue, low). (1a) The 393 most significantly differentially expressed genes in the training set organized by hierarchical clustering. (1b) The same 393 transcript list, ordered in the same gene tree, was used to analyse the data from the independent Test Set, with hierarchical clustering by Spearman correlation with average linkage creating a condition tree (along the upper horizontal edge of the heatmap) and the study grouping (i.e. the clinical phenotype) presented as coloured blocks at the base of each profile. (1c) The independent Validation Set recruited in South Africa was analysed as above.

FIGS. 2a to 2c: The transcriptional signature of active TB correlates with the radiographic extent of disease. Chest radiographs for each patient in the Training and independent Test Sets were assessed by three independent clinicians (FIG. 9a) blinded to other data. (2a) The 393 transcript profiles are shown for each patient with active TB in the independent Test Set. Representative radiographic examples of Advanced disease, Moderate disease, Minimal disease and No disease are illustrated. (2b) Depicted are XRay Grade and Transcriptional Perturbation in relation to transcriptional variance and XRay score. (2c) Profiles were grouped according to radiographic extent of disease and the mean “Molecular Distance to Health” (Additional Methods) for each group compared using Kruskal-Wallis ANOVA, with Dunn's multiple comparison post hoc testing to compare between groups (***=p <0.0001).

FIGS. 3a to 3d. The transcriptional signature of active TB is diminished during successful treatment. (3a) 7 patients with active TB (Active) were re-sampled at 2 and 12 months following the initiation of anti-mycobacterial treatment and compared with healthy controls from the independent Test Set (Control, n=12). (3b) Chest radiographs at the time of diagnosis and 2 and 12 months following the initiation of anti-mycobacterial treatment, are shown for 2 of the 7 patients (labelled “4” or “7”). Profiles for these individuals are shown above marked by the same numerical indicator. (3c) “Molecular Distance to Health” for each patient was calculated at each timepoint and compared with time post initiation of treatment using Spearman correlation. (3d) The mean “Molecular Distance to Health” for each timepoint was compared using Friedman's test, with Dunn's multiple comparison post-hoc testing to compare between timepoints. Horizontal bars indicate the median, 5^{th} and 95^{th} percentiles.

FIGS. 4a to 4e. The whole blood transcriptional signature of active TB reflects both distinct changes in cellular composition and changes in the absolute levels of gene expression. (4a) Gene expression of active TB compared with healthy controls are mapped within a pre-defined modular framework. The intensity of the spot represents the proportion of significantly differentially expressed transcripts for each module (red=increased, blue=decreased, transcript abundance). Functional interpretations previously determined by unbiased literature profiling are indicated by the colour coded grid below (4b) Whole blood from Test Set healthy controls (Control) and active TB patients (Active) analysed by flow cytometry for CD3⁺CD4⁺ and CD3⁺CD8⁺ T cells and CD19⁺CD20⁺ B cells. Error bars=median. (4c) Whole blood from Test Set healthy controls (Control) and active TB patients (Active) analysed by flow cytometry for CD 14⁺ monocytes, CD14⁺CD16⁺ inflammatory monocytes and CD 16⁺ neutrophils. Error bars=median. (4d) The Ingenuity Pathways analysis canonical pathway for interferon signalling is displayed here with each gene product identified with a symbol corresponding to its function (legend on right) and transcripts over-represented in the Training Set active TB patients are shaded red. (4e) Serum levels of CXCL10 (IP10) from healthy controls (Control) and patients with active pulmonary TB (Active). Statistical comparison was performed using two-tailed Mann-Whitney test. The horizontal bar indicates the mean for each group, with the whiskers indicating the 95% confidence interval.

FIGS. 5a and 5b. Interferon-inducible gene expression in active TB. Interferon-inducible gene (5a) transcript abundance in whole blood samples from active TB (Training, Test and Validation Sets); and (5b) expression in separated blood leucocyte populations from Test Set blood. Gene abundance/expression is shown as compared to the median of the healthy controls (labelled as in FIG. 1). Numbers shown in the Test Set and the separated populations correspond to individual patients.

FIGS. 6a to 6d. PDL1 (CD274) is overabundant in whole blood of patients with active TB, predominantly due to its overexpression by neutrophils. (6a) Abundance of PDL1 (normalized to the median of all samples) in whole blood of active TB patients (Active) and healthy controls (Control) (or Latent South Africa). Also shown is the geometric mean fluorescence intensity (MFI) of PDL1 on whole blood leucocytes from a representative patient and control. MFI levels are linked to expression profiles for PDL1 by arrows. Graph shows pooled MFI data from 11 11 active TB patients and 11 health controls (error bars=mean±95% CI). (6b) The MFI of PDL1 on different cell sub-populations (blue), compared to PDL1 on total leucocytes (red) and isotype control of the total cells (green). Shown are a control and a patient. Graphs show pooled MFI data from the same number of active TB patients and healthy controls (error bars=mean±95% CI). (6c) The expression for PDL1, normalized to the median of all samples, is shown for 4 controls and 7 active TB patients in enriched cell sub-populations. (6d) The abundance of PDL1 in the whole blood of 7 patients with active TB (Active) is shown at 0, 2 and 12 months post anti-mycobacterial treatment, compared with 12 healthy controls from the Test Set (Control).

FIGS. 7a to 7c. Formation of the Training, Test and Validation Sets. Each cohort was not only independently recruited, but all stages of RNA processing and microarray analysis were also performed completely independently. (7a) The recruitment of the Training Set cohort in London, UK; (7b) The recruitment of the independent Test Set cohort in London, UK. (7c) The recruitment of the independent Validation Set cohort in Cape Town, South Africa.

FIGS. 8a to 8e. Hierarchical clustering of patient profiles. (8a) The 1836 transcript expression profiles for the Training Set were subjected to unsupervised hierarchical clustering by Spearman correlation with average linkage to create a condition tree (along the upper edge of the heatmap). These patient clusters can then be compared with the clinical and demographic parameters displayed in blocks underneath each profile along the lower edge of the heatmap. A key is provided at the bottom of the figure. Clusters were divided evenly according to distance. (8b) The 393 transcript expression profiles for the Test Set clustered by Pearson correlation with average linkage. (8c) The 393 transcript expression profiles for the validation set clustered by Pearson correlation with average linkage. (8d) The 393 transcript patient expression profiles for only those aged 22 to 34 years old in the Validation Set. (8e) The 393 transcript patient expression profiles for only those aged 22 to 34 years old in the Validation Set.

FIGS. 9a to 9c. A comparison of the transcriptional signature of Active TB with the radiographic extent of disease. (9a) The classification scheme used to grade chest radiographs according to extent of disease. (9b) The 393 transcript expression profiles for all 13 Active TB patients in the Training Set, along with their corresponding chest radiograph taken at the time of diagnosis, with both grouped according to X-ray Grade as per the classification scheme. The expression profile and radiograph of a given patient is given the same numerical indicator. (9c) The 393 transcript expression profiles and chest radiographs for the 21 Active TB patients in the Test Set.

FIGS. 10a to 10d. The whole blood transcriptional signature of active TB reflects both distinct changes in cellular composition and changes in the absolute levels of gene expression. Gene expression of active TB compared with healthy controls are mapped within a pre-defined modular framework. The intensity of the spot represents the proportion of significantly differentially expressed transcripts for each module (red=increased, blue=decreased, transcript abundance). Functional interpretations previously determined by unbiased literature profiling are indicated by the colour coded grid in main FIG. 4. Here is demonstrated the percentage of genes in each module that is over- (red) or under-represented (blue) in the (10a) Training Set; (10b) Test Set; (10c) Validation Set (SA). (10d) The weighted molecular distance to health was calculated for each patient at baseline pre-treatment (0 months), and at 2 and 12 months following the initiation of anti-mycobacterial therapy. The individual patient numbers correspond to those shown in FIGS. 3a to 3d.

FIGS. 11a to 11c. Analysis of lymphocytes in blood of active TB patients and controls. (11a) Shown are flow cytometric gating strategies used to analyse whole blood from Test Set healthy controls and active TB patients for T cells and B cells. The top row of panels shows the backgating strategy used to determine the lymphocyte FSC/SSC gate used in subsequent gating. A large FSC/SSC gate was set initially (left panel) and then analysed for CD45 vs CD3. CD45CD3 cells were gated (middle panel) and their FSC/SSC profile determined (right panel). This profile was then used to determine an appropriate lymphocyte FSC/SSC gate (see second row, left hand panel). This backgating procedure was also carried out gating on CD45⁺CD19⁺ (B cells) to ensure these cells were included in the lymphocyte gate (not shown). The second row of panels shows the gating strategy used to identify T cell populations. A lymphocyte FSC/SSC gate was set and these cells assessed for CD45 vs CD3 (2^{nd} panel from left). CD45⁺ cells were then gated and assessed for CD3 vs CD8. CD3⁺ T cells were gated and assessed for CD4 and CD8 expression. CD4⁺ and CD8⁺ subsets were then gated. Rows 3-6 show the gating strategy used to define T cell memory subsets. CD4 and CD8 T cells gated as in row 2 were assessed for CD45RA vs CCR7 expression and a quadrant set based on isotype controls (rows 5 & 6) to define naïve (CD45RA⁺CCR7⁺), central memory (CD45RA-CCR7⁺), effector memory (CD45RA⁻CCR7⁻) and in the case of CD8⁺ T cells, terminally differentiated effector (CD45RA⁺CCR7⁻) T cells. These subsets were also assessed for CD62L expression. The bottom row of panels shows the strategy used to gate B cells. A lymphocyte FSC/SSC gate was set and cells assessed for CD45 vs CD19. CD45⁺ cells were gated and assessed for CD19 and CD20. B cells were defined as CD19⁺CD20⁺. (11b) Whole blood from 11 test set healthy controls (Control) and 9 test set active TB patients (Active) was analysed by multi-parameter flow cytometry for T cell memory populations. Full flow cytometry gating strategy is shown in FIG. 11a. Graphs show pooled data of all individuals for percentages of naïve, central memory (TCM), effector memory (TEM) and terminally differentiated effector (TD, CD8⁺ T cells only) cell subsets (top row, each group) and cell numbers (×10⁶/ml) for each cell subset (bottom row, each group). Each symbol represents an individual patient. Horizontal line represents the median. (11c) Gene (i) T cell transcript abundance in whole blood samples from active TB (Training, Test and Validation Sets); and (ii) expression in separated blood leucocyte populations from Test Set blood. Gene abundance/expression is shown as compared to the median of the healthy controls (labelled as in FIG. 1). Numbers shown in the Test Set and the separated populations correspond to individual patients.

FIGS. 12a to 12c. Analysis of myeloid cells in blood of active TB patients and controls. (12a) Shown are flow cytometric gating strategies used to analyse whole blood from test set healthy controls and active TB patients for monocytes and neutrophils. A large FSC/SSC gate was set (top row, left panel) and was then analysed for CD45 vs CD14. CD45′ cells were gated (middle panel) and assessed for CD14 vs CD16. Monocytes were defined as CD 14⁺, inflammatory monocytes as CD ‘CD16’ and neutrophils as CD16⁺. Also shown in this figure is the gating strategy used to assess possible overlap between CD16′ neutrophils and CD16 expressing NK cells. A large FSC/SSC gate was set to encompass both neutrophils and NK cells. CD45′ cells were then assessed for CD16 vs CD56 (NK cell marker). CD16′ neutrophils expressed high levels of CD 16 and not CD56 (as shown by isotype control plot, bottom panel). CD56′ NK cells expressed intermediate levels of CD16 and did not overlap with CD16hi cells. CD56′CD16int cells and CD16hi cells had different FSC/SSC properties. (12b) Myeloid gene (i) transcript abundance in whole blood samples from active TB (Training, Test and Validation Sets); and (ii) expression in separated blood leucocyte populations from Test Set blood. Gene abundance/expression is shown as compared to the median of the healthy controls (labelled as in FIG. 1). Numbers shown in the Test Set and the separated populations correspond to individual patients. (12c) (i) T cell transcript abundance in whole blood samples from active TB (Training, Test and Validation Sets); and (ii) expression in separated blood leucocyte populations from Test Set whole blood. Gene abundance/expression is shown as compared to the median of the healthy controls. The expression profiles of a given patient are given the same numerical indicator.

FIGS. 13a to 13c. Ingenuity Pathways analysis of the 393-transcript signature. (13a) The probability (as a-log of the p-value calculated by Fischer's Exact test, with Benjamini-Hochberg multiple testing correction) that each canonical biological pathway is significantly over-represented is indicated by the orange squares. The solid coloured bars represent the percentage of the total number of genes comprising that pathway (given in bold at the right hand edge of each bar) present in the analysed gene list. The colour of the bar indicates the abundance of those transcripts in the whole blood of patients with Active TB compared with healthy controls in the training set. (13b) Serum levels of interferon-alpha 2a (IFN-α 2a), and (13c) interferon-gamma (IFN-γ) are shown here for the 12 healthy controls and 13 patients with Active TB used for the training set microarray analyses. No significant difference was observed between groups for either cytokine using two-tailed Mann-Whitney test. The horizontal line indicates the mean for each group and the whiskers indicate the 95% confidence interval.

FIGS. 14a to 14f. The Test Set (UK) and the 1836 list as well as the 393 list for the control (12) as well as active (21).

FIGS. 15a and 15b1-15b4. PDL1 (CD274) expression on whole blood and cell sub-populations from individual healthy controls and patients with active TB. (15a) (4-40 Whole blood from 11 Test Set healthy controls (Control) and 11 Test Set active TB patients (Active) was analysed by flow cytometry for expression of PDL 1. A large FSC/SSC gate was set to encompass total white blood cells and the geometric mean fluorescence intensity (MFI) of PDL1 (in red) as compared to isotype control (green) assessed. Each active TB patient was analysed on a different day, healthy controls were analysed in small groups (from left, samples 1 & 2, 3 & 4, 6-8 and 9-11 were run together, 5 was run singly) and samples within each group share an isotype control. (15b1-15b4) (4-410-Cell sub-populations from the blood of the same 11 Test Set healthy controls (Control) and 11 Test Set active TB patients (Active) as in part a. were also analysed by flow cytometry for expression of PDL 1. Cell sub-populations were defined as in FIG. 6b. and MFIs of PDL 1 (in red) as compared to isotype control (green) plotted.

FIGS. 16a to 16f. The Training Set 393-transcript profiles ordered according to study group are shown magnified with gene symbols are listed at the right of the figure. Key transcripts are highlighted by larger text. At the left of each figure the entire gene tree and heatmap is displayed, with the enlarged area marked by a black rectangle. The relative abundance of transcripts is indicated by a colour scale at the base of the figure (as in FIG. 1).

FIGS. 17A-B are tables of activity profiles of subjects.

FIG. 18 is a table of the activity profiles of subjects.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2d ed. 1994); The Cambridge Dictionary of Science and Technology (Walker ed., 1988); The Glossary of Genetics, 5TH ED., R. Rieger et al. (eds.), Springer Verlag (1991); and Hale & Marham, The Harper Collins Dictionary of Biology (1991).

Various biochemical and molecular biology methods are well known in the art. For example, methods of isolation and purification of nucleic acids are described in detail in WO 97/10365; WO 97/27317; Chapter 3 of Laboratory Techniques in Biochemistry and Molecular Biology: Hybridization with Nucleic Acid Probes, Part I. Theory and Nucleic Acid Preparation, (P. Tijssen, ed.) Elsevier, N.Y. (1993); Sambrook, et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements.

### BIOINFORMATICS DEFINITIONS

As used herein, an “object” refers to any item or information of interest (generally textual, including noun, verb, adjective, adverb, phrase, sentence, symbol, numeric characters, etc.). Therefore, an object is anything that can form a relationship and anything that can be obtained, identified, and/or searched from a source. “Objects” include, but are not limited to, an entity of interest such as gene, protein, disease, phenotype, mechanism, drug, etc. In some aspects, an object may be data, as further described below.

As used herein, a “relationship” refers to the co-occurrence of objects within the same unit (e.g., a phrase, sentence, two or more lines of text, a paragraph, a section of a webpage, a page, a magazine, paper, book, etc.). It may be text, symbols, numbers and combinations, thereof

As used herein, “meta data content” refers to information as to the organization of text in a data source. Meta data can comprise standard metadata such as Dublin Core metadata or can be collection-specific. Examples of metadata formats include, but are not limited to, Machine Readable Catalog (MARC) records used for library catalogs, Resource Description Format (RDF) and the Extensible Markup Language (XML). Meta objects may be generated manually or through automated information extraction algorithms.

As used herein, an “engine” refers to a program that performs a core or essential function for other programs. For example, an engine may be a central program in an operating system or application program that coordinates the overall operation of other programs. The term “engine” may also refer to a program containing an algorithm that can be changed. For example, a knowledge discovery engine may be designed so that its approach to identifying relationships can be changed to reflect new rules of identifying and ranking relationships.

As used herein, “semantic analysis” refers to the identification of relationships between words that represent similar concepts, e.g., though suffix removal or stemming or by employing a thesaurus. “Statistical analysis” refers to a technique based on counting the number of occurrences of each term (word, word root, word stem, n-gram, phrase, etc.). In collections unrestricted as to subject, the same phrase used in different contexts may represent different concepts. Statistical analysis of phrase co-occurrence can help to resolve word sense ambiguity. “Syntactic analysis” can be used to further decrease ambiguity by part-of-speech analysis. As used herein, one or more of such analyses are referred to more generally as “lexical analysis.” “Artificial intelligence (AI)” refers to methods by which a non-human device, such as a computer, performs tasks that humans would deem noteworthy or “intelligent.” Examples include identifying pictures, understanding spoken words or written text, and solving problems.

Terms such “data”, “dataset” and “information” are often used interchangeably, as are “information” and “knowledge.” As used herein, “data” is the most fundamental unit that is an empirical measurement or set of measurements. Data is compiled to contribute to information, but it is fundamentally independent of it and may be combined into a dataset, that is, a set of data. Information, by contrast, is derived from interests, e.g., data (the unit) may be gathered on ethnicity, gender, height, weight and diet for the purpose of finding variables correlated with risk of cardiovascular disease. However, the same data could be used to develop a formula or to create “information” about dietary preferences, i.e., likelihood that certain products in a supermarket have a higher likelihood of selling.

As used herein, the term “database” refers to repositories for raw or compiled data, even if various informational facets can be found within the data fields. A database may include one or more datasets. A database is typically organized so its contents can be accessed, managed, and updated (e.g., the database is dynamic). The term “database” and “source” are also used interchangeably in the present invention, because primary sources of data and information are databases. However, a “source database” or “source data” refers in general to data, e.g., unstructured text and/or structured data that are input into the system for identifying objects and determining relationships. A source database may or may not be a relational database. However, a system database usually includes a relational database or some equivalent type of database which stores values relating to relationships between objects.

As used herein, a “system database” and “relational database” are used interchangeably and refer to one or more collections of data organized as a set of tables containing data fitted into predefined categories. For example, a database table may comprise one or more categories defined by columns (e.g. attributes), while rows of the database may contain a unique object for the categories defined by the columns. Thus, an object such as the identity of a gene might have columns for its presence, absence and/or level of expression of the gene. A row of a relational database may also be referred to as a “set” and is generally defined by the values of its columns. A “domain” in the context of a relational database is a range of valid values a field such as a column may include.

As used herein, a “domain of knowledge” refers to an area of study over which the system is operative, for example, all biomedical data. It should be pointed out that there is advantage to combining data from several domains, for example, biomedical data and engineering data, for this diverse data can sometimes link things that cannot be put together for a normal person that is only familiar with one area or research/study (one domain). A “distributed database” refers to a database that may be dispersed or replicated among different points in a network.

As used herein, “information” refers to a data set that may include numbers, letters, sets of numbers, sets of letters, or conclusions resulting or derived from a set of data. “Data” is then a measurement or statistic and the fundamental unit of information. “Information” may also include other types of data such as words, symbols, text, such as unstructured free text, code, etc. “Knowledge” is loosely defined as a set of information that gives sufficient understanding of a system to model cause and effect. To extend the previous example, information on demographics, gender and prior purchases may be used to develop a regional marketing strategy for food sales while information on nationality could be used by buyers as a guideline for importation of products. It is important to note that there are no strict boundaries between data, information, and knowledge; the three terms are, at times, considered to be equivalent. In general, data comes from examining, information comes from correlating, and knowledge comes from modeling.

As used herein, “a program” or “computer program” refers generally to a syntactic unit that conforms to the rules of a particular programming language and that is composed of declarations and statements or instructions, divisible into, “code segments” needed to solve or execute a certain function, task, or problem. A programming language is generally an artificial language for expressing programs.

As used herein, a “system” or a “computer system” generally refers to one or more computers, peripheral equipment, and software that perform data processing. A “user” or “system operator” in general includes a person, that uses a computer network accessed through a “user device” (e.g., a computer, a wireless device, etc) for the purpose of data processing and information exchange. A “computer” is generally a functional unit that can perform substantial computations, including numerous arithmetic operations and logic operations without human intervention.

As used herein, “application software” or an “application program” refers generally to software or a program that is specific to the solution of an application problem. An “application problem” is generally a problem submitted by an end user and requiring information processing for its solution.

As used herein, a “natural language” refers to a language whose rules are based on current usage without being specifically prescribed, e.g., English, Spanish or Chinese. As used herein, an “artificial language” refers to a language whose rules are explicitly established prior to its use, e.g., computer-programming languages such as C, C++, Java, BASIC, FORTRAN, or COBOL.

As used herein, “statistical relevance” refers to using one or more of the ranking schemes (0/E ratio, strength, etc.), where a relationship is determined to be statistically relevant if it occurs significantly more frequently than would be expected by random chance.

As used herein, the terms “coordinately regulated genes” or “transcriptional modules” are used interchangeably to refer to grouped, gene expression profiles (e.g., signal values associated with a specific gene sequence) of specific genes. Each transcriptional module correlates two key pieces of data, a literature search portion and actual empirical gene expression value data obtained from a gene microarray. The set of genes that is selected into a transcriptional modules is based on the analysis of gene expression data (module extraction algorithm described above). Additional steps are taught by Chaussabel, D. & Sher, A. Mining microarray expression data by literature profiling. Genome Biol 3, RESEARCH0055 (2002), (http://genomebiology.com/2002/3/10/research/0055) relevant portions incorporated herein by reference and expression data obtained from a disease or condition of interest, e.g., Systemic Lupus erythematosus, arthritis, lymphoma, carcinoma, melanoma, acute infection, autoimmune disorders, autoinflammatory disorders, etc.).

The Table below lists examples of keywords that were used to develop the literature search portion or contribution to the transcription modules. The skilled artisan will recognize that other terms may easily be selected for other conditions, e.g., specific cancers, specific infectious disease, transplantation, etc. For example, genes and signals for those genes associated with T cell activation are described hereinbelow as Module ID “M 2.8” in which certain keywords (e.g., Lymphoma, T-cell, CD4, CD8, TCR, Thymus, Lymphoid, IL2) were used to identify key T-cell associated genes, e.g., T-cell surface markers (CD5, CD6, CD7, CD26, CD28, CD96); molecules expressed by lymphoid lineage cells (lymphotoxin beta, IL2-inducible T-cell kinase, TCF7; and T-cell differentiation protein mal, GATA3, STAT5B). Next, the complete module is developed by correlating data from a patient population for these genes (regardless of platform, presence/absence and/or up or downregulation) to generate the transcriptional module. In some cases, the gene profile does not match (at this time) any particular clustering of genes for these disease conditions and data, however, certain physiological pathways (e.g., cAMP signaling, zinc-finger proteins, cell surface markers, etc.) are found within the “Underdetermined” modules. In fact, the gene expression data set may be used to extract genes that have coordinated expression prior to matching to the keyword search, i.e., either data set may be correlated prior to cross-referencing with the second data set.

TABLE 1
Transcriptional Modules

| Example | | |
|---|---|---|
| Module | Example Keyword | |
| I.D. | selection | Gene Profile Assessment |
| M 1.1 | Ig, Immunoglobulin, | Plasma cells: Includes genes encoding for Immunoglobulin |
| | Bone, Marrow, PreB, | chains (e.g. IGHM, IGJ, IGLL1, IGKC, IGHD) and the |
| | IgM, Mu. | plasma cell marker CD38. |
| M 1.2 | Platelet, Adhesion, | Platelets: Includes genes encoding for platelet glycoproteins |
| | Aggregation, Endothelial, | (ITGA2B, ITGB3, GP6, GP1A/B), and platelet-derived |
| | Vascular | immune mediators such as PPPB (pro-platelet basic protein) |
| | | and PF4 (platelet factor 4). |
| M 1.3 | Immunoreceptor, BCR, | B-cells: Includes genes encoding for B-cell surface markers |
| | B-cell, IgG | (CD72, CD79A/B, CD19, CD22) and other B-cell associated |
| | | molecules: Early B-cell factor (EBF), B-cell linker (BLNK) |
| | | and B lymphoid tyrosine kinase (BLK). |
| M 1.4 | Replication, Repression, | Undetermined. This set includes regulators and targets of |
| | Repair, CREB, | cAMP signaling pathway (JUND, ATF4, CREM, PDE4, |
| | Lymphoid, TNF-alpha | NR4A2, VIL2), as well as repressors of TNF-alpha mediated |
| | | NF-KB activation (CYLD, ASK, TNFAIP3). |
| M 1.5 | Monocytes, Dendritic, | Myeloid lineage: Includes molecules expressed by cells of |
| | MHC, Costimulatory, | the myeloid lineage (CD86, CD163, FCGR2A), some of |
| | TLR4, MYD88 | which being involved in pathogen recognition (CD14, TLR2, |
| | | MYD88). This set also includes TNF family members |
| | | (TNFR2, BAFF). |
| M 1.6 | Zinc, Finger, P53, RAS | Undetermined. This set includes genes encoding for |
| | | signaling molecules, e.g., the zinc finger containing inhibitor |
| | | of activated STAT (PIAS1 and PIAS2), or the nuclear factor |
| | | of activated T-cells NFATC3. |
| M 1.7 | Ribosome, Translational, | MHC/Ribosomal proteins: Almost exclusively formed by |
| | 40S, 60S, HLA | genes encoding MHC class I molecules (HLA-A,B,C,G,E) + |
| | | Beta 2-microglobulin (B2M) or Ribosomal proteins (RPLs, |
| | | RPSs). |
| M 1.8 | Metabolism, | Undetermined. Includes genes encoding metabolic enzymes |
| | Biosynthesis, Replication, | (GLS, NSF1, NAT1) and factors involved in DNA |
| | Helicase | replication (PURA, TERF2, EIF2S1). |
| M 2.1 | NK, Killer, Cytolytic, | Cytotoxic cells: Includes cytotoxic T-cells and NK-cells |
| | CD8, Cell-mediated, T- | surface markers (CD8A, CD2, CD160, NKG7, KLRs), |
| | cell, CTL, IFN-g | cytolytic molecules (granzyme, perforin, granulysin), |
| | | chemokines (CCL5, XCL1) and CTL/NK-cell associated |
| | | molecules (CTSW). |
| M 2.2 | Granulocytes, | Neutrophils: This set includes innate molecules that are |
| | Neutrophils, Defense, | found in neutrophil granules (Lactotransferrin: LTF, |
| | Myeloid, Marrow | defensin: DEAF1, Bacterial Permeability Increasing protein: |
| | | BPI, Cathelicidin antimicrobial protein: CAMP). |
| M 2.3 | Erythrocytes, Red, | Erythrocytes: Includes hemoglobin genes (HGBs) and other |
| | Anemia, Globin, | erythrocyte-associated genes (erythrocytic alkirin: ANK1, |
| | Hemoglobin | Glycophorin C: GYPC, hydroxymethylbilane synthase: |
| | | HMBS, erythroid associated factor: ERAF). |
| M 2.4 | Ribonucleoprotein, 60S, | Ribosomal proteins: Including genes encoding ribosomal |
| | nucleolus, Assembly, | proteins (RPLs, RPSs), Eukaryotic Translation Elongation |
| | Elongation | factor family members (EEFs) and Nucleolar proteins |
| | | (NPM1, NOAL2, NAP1L1). |
| M 2.5 | Adenoma, Interstitial, | Undetermined. This module includes genes encoding |
| | Mesenchyme, Dendrite, | immune-related (CD40, CD80, CXCL12, IFNA5, IL4R) as |
| | Motor | well as cytoskeleton-related molecules (Myosin, Dedicator |
| | | of Cytokenesis, Syndecan 2, Plexin C1, Distrobrevin). |
| M 2.6 | Granulocytes, Monocytes, | Myeloid lineage: Related to M 1.5. Includes genes expressed |
| | Myeloid, ERK, Necrosis | in myeloid lineage cells (IGTB2/CD18, Lymphotoxin beta |
| | | receptor, Myeloid related proteins 8/14 Formyl peptide |
| | | receptor 1), such as Monocytes and Neutrophils: |
| M 2.7 | No keywords extracted. | Undetermined. This module is largely composed of |
| | | transcripts with no known function. Only 20 genes |
| | | associated with literature, including a member of the |
| | | chemokine-like factor superfamily (CKLFSF8). |
| M 2.8 | Lymphoma, T-cell, CD4, | T-cells: Includes T-cell surface markers (CD5, CD6, CD7, |
| | CD8, TCR, Thymus, | CD26, CD28, CD96) and molecules expressed by lymphoid |
| | Lymphoid, IL2 | lineage cells (lymphotoxin beta, IL2-inducible T-cell kinase, |
| | | TCF7, T-cell differentiation protein mal, GATA3, |
| | | STAT5B). |
| M 2.9 | ERK, Transactivation, | Undetermined. Includes genes encoding molecules that |
| | Cytoskeletal, MAPK, | associate to the cytoskeleton (Actin related protein 2/3, |
| | JNK | MAPK1, MAP3K1, RAB5A). Also present are T-cell |
| | | expressed genes (FAS, ITGA4/CD49D, ZNF1A1). |
| M 2.10 | Myeloid, Macrophage, | Undetermined. Includes genes encoding for Immune-related |
| | Dendritic, Inflammatory, | cell surface molecules (CD36, CD86, LILRB), cytokines |
| | Interleukin | (IL15) and molecules involved in signaling pathways (FYB, |
| | | TICAM2-Toll-like receptor pathway). |
| M 2.11 | Replication, Repress, | Undetermined. Includes kinases (UHMK1, CSNK1G1, |
| | RAS, | CDK6, WNK1, TAOK1, CALM2, PRKCI, ITPKB, SRPK2, |
| | Autophosphorylation, | STK17B, DYRK2, PIK3R1, STK4, CLK4, PKN2) and RAS |
| | Oncogenic | family members (G3BP, RAB14, RASA2, RAP2A, KRAS). |
| M 3.1 | ISRE, Influenza, | Interferon-inducible: This set includes interferon-inducible |
| | Antiviral, IFN-gamma, | genes: antiviral molecules (OAS1/2/3/L, GBP1, G1P2, |
| | IFN-alpha, Interferon | EIF2AK2/PKR, MX1, PML), chemokines (CXCL10/IP-10), |
| | | signaling molecules (STAT1, STAt2, IRF7, ISGF3G). |
| M 3.2 | TGF-beta, TNF, | Inflammation I: Includes genes encoding molecules |
| | Inflammatory, Apoptotic, | involved in inflammatory processes (e.g., IL8, ICAM1, |
| | Lipopolysaccharide | C5R1, CD44, PLAUR, IL1A, CXCL16), and regulators of |
| | | apoptosis (MCL1, FOXO3A, RARA, BCL3/6/2A1, |
| | | GADD45B). |
| M 3.3 | Granulocyte, | Inflammation II: Includes molecules inducing or inducible |
| | Inflammatory, Defense, | by Granulocyte-Macrophage CSF (SPI1, IL18, ALOX5, |
| | Oxidize, Lysosomal | ANPEP), as well as lysosomal enzymes (PPT1, CTSB/S, |
| | | CES1, NEU1, ASAH1, LAMP2, CAST). |
| M 3.4 | No keyword extracted | Undetermined. Includes protein phosphates (PPP1R12A, |
| | | PTPRC, PPP1CB, PPM1B) and phosphoinositide 3-kinase |
| | | (PI3K) family members (PIK3CA, PIK32A, PIP5K3). |
| M 3.5 | No keyword extracted | Undetermined. Composed of only a small number of |
| | | transcripts. Includes hemoglobin genes (HBA1, HBA2, |
| | | HBB). |
| M 3.6 | Complement, Host, | Undetermined. Large set that includes T-cell surface markers |
| | Oxidative, Cytoskeletal, | (CD101, CD102, CD103) as well as molecules ubiquitously |
| | T-cell | expressed among blood leukocytes (CXRCR1: fraktalkine |
| | | receptor, CD47, P-selectin ligand). |
| M 3.7 | Spliceosome, | Undetermined. Includes genes encoding proteasome subunits |
| | Methylation, Ubiquitin, | (PSMA2/5, PSMB5/8); ubiquitin protein ligases HIP2, |
| | Beta-catenin | STUB1, as well as components of ubiqutin ligase complexes |
| | | (SUGT1). |
| M 3.8 | CDC, TCR, CREB, | Undetermined. Includes genes encoding for several enzymes: |
| | Glycosylase | aminomethyltransferase, arginyltransferase, asparagines |
| | | synthetase, diacylglycerol kinase, inositol phosphatases, |
| | | methyltransferases, helicases . . . |
| M 3.9 | Chromatin, Checkpoint, | Undetermined. Includes genes encoding for protein kinases |
| | Replication, | (PRKPIR, PRKDC, PRKCI) and phosphatases (e.g., PTPLB, |
| | Transactivation | PPP1R8/2CB). Also includes RAS oncogene family |
| | | members and the NK cell receptor 2B4 (CD244). |

### BIOLOGICAL DEFINITIONS

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays” or “gene-chips” that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the sample by measuring the abundance and/or activity of cellular constituents, characterizing according to morphological phenotype or a combination of the methods for the detection of transcripts.

As used herein, the term “expression profile” refers to the relative abundance of RNA, DNA or protein abundances or activity levels. The expression profile can be a measurement for example of the transcriptional state or the translational state by any number of methods and using any of a number of gene-chips, gene arrays, beads, multiplex PCR, quantitiative PCR, run-on assays, Northern blot analysis, Western blot analysis, protein expression, fluorescence activated cell sorting (FACS), enzyme linked immunosorbent assays (ELISA), chemiluminescence studies, enzymatic assays, proliferation studies or any other method, apparatus and system for the determination and/or analysis of gene expression that are readily commercially available.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

As used herein, the term “modular transcriptional vectors” refers to transcriptional expression data that reflects the “proportion of differentially expressed genes.” For example, for each module the proportion of transcripts differentially expressed between at least two groups (e.g. healthy subjects vs patients). This vector is derived from the comparison of two groups of samples. The first analytical step is used for the selection of disease-specific sets of transcripts within each module. Next, there is the “expression level.” The group comparison for a given disease provides the list of differentially expressed transcripts for each module. It was found that different diseases yield different subsets of modular transcripts. With this expression level it is then possible to calculate vectors for each module(s) for a single sample by averaging expression values of disease-specific subsets of genes identified as being differentially expressed. This approach permits the generation of maps of modular expression vectors for a single sample, e.g., those described in the module maps disclosed herein. These vector module maps represent an averaged expression level for each module (instead of a proportion of differentially expressed genes) that can be derived for each sample.

Using the present invention it is possible to identify and distinguish diseases not only at the module-level, but also at the gene-level; i.e., two diseases can have the same vector (identical proportion of differentially expressed transcripts, identical “polarity”), but the gene composition of the vector can still be disease-specific. Gene-level expression provides the distinct advantage of greatly increasing the resolution of the analysis. Furthermore, the present invention takes advantage of composite transcriptional markers. As used herein, the term “composite transcriptional markers” refers to the average expression values of multiple genes (subsets of modules) as compared to using individual genes as markers (and the composition of these markers can be disease-specific). The composite transcriptional markers approach is unique because the user can develop multivariate microarray scores to assess disease severity in patients with, e.g., SLE, or to derive expression vectors disclosed herein. Most importantly, it has been found that using the composite modular transcriptional markers of the present invention the results found herein are reproducible across microarray platform, thereby providing greater reliability for regulatory approval.

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and modules of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. The modules of the present invention allow for the first time the design of simple, custom arrays that provide optimal data with the least number of probes while maximizing the signal to noise ratio. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, beads (e.g., Luminex), multiplex PCR, quantitiative PCR, run-on assays, Northern blot analysis, or even, for protein analysis, e.g., Western blot analysis, 2-D and 3-D gel protein expression, MALDI, MALDI-TOF, fluorescence activated cell sorting (FACS) (cell surface or intracellular), enzyme linked immunosorbent assays (ELISA), chemiluminescence studies, enzymatic assays, proliferation studies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or downregulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

For some disease states it is possible to identify cellular or morphological differences, especially at early levels of the disease state. The present invention avoids the need to identify those specific mutations or one or more genes by looking at modules of genes of the cells themselves or, more importantly, of the cellular RNA expression of genes from immune effector cells that are acting within their regular physiologic context, that is, during immune activation, immune tolerance or even immune anergy. While a genetic mutation may result in a dramatic change in the expression levels of a group of genes, biological systems often compensate for changes by altering the expression of other genes. As a result of these internal compensation responses, many perturbations may have minimal effects on observable phenotypes of the system but profound effects to the composition of cellular constituents. Likewise, the actual copies of a gene transcript may not increase or decrease, however, the longevity or half-life of the transcript may be affected leading to greatly increases protein production. The present invention eliminates the need of detecting the actual message by, in one embodiment, looking at effector cells (e.g., leukocytes, lymphocytes and/or sub-populations thereof) rather than single messages and/or mutations.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms; one or more module-level analytical processes; the characterization of blood leukocyte transcriptional modules; the use of aggregated modular data in multivariate analyses for the molecular diagnostic/prognostic of human diseases; and/or visualization of module-level data and results. Using the present invention it is also possible to develop and analyze composite transcriptional markers, which may be further aggregated into a single multivariate score.

An explosion in data acquisition rates has spurred the development of mining tools and algorithms for the exploitation of microarray data and biomedical knowledge. Approaches aimed at uncovering the modular organization and function of transcriptional systems constitute promising methods for the identification of robust molecular signatures of disease. Indeed, such analyses can transform the perception of large scale transcriptional studies by taking the conceptualization of microarray data past the level of individual genes or lists of genes.

The present inventors have recognized that current microarray-based research is facing significant challenges with the analysis of data that are notoriously “noisy,” that is, data that is difficult to interpret and does not compare well across laboratories and platforms. A widely accepted approach for the analysis of microarray data begins with the identification of subsets of genes differentially expressed between study groups. Next, the users try subsequently to “make sense” out of resulting gene lists using pattern discovery algorithms and existing scientific knowledge.

Rather than deal with the great variability across platforms, the present inventors have developed a strategy that emphasized the selection of biologically relevant genes at an early stage of the analysis. Briefly, the method includes the identification of the transcriptional components characterizing a given biological system for which an improved data mining algorithm was developed to analyze and extract groups of coordinately expressed genes, or transcriptional modules, from large collections of data.

Pulmonary tuberculosis (PTB) is a major and increasing cause of morbidity and mortality worldwide caused by Mycobacterium tuberculosis (M. tuberculosis). However, the majority of individuals infected with M. tuberculosis remain asymptomatic, retaining the infection in a latent form and it is thought that this latent state is maintained by an active immune response. Blood is the pipeline of the immune system, and as such is the ideal biologic material from which the health and immune status of an individual can be established. Here, using microarray technology to assess the activity of the entire genome in blood cells, we identified distinct and reciprocal blood transcriptional biomarker signatures in patients with active pulmonary tuberculosis and latent tuberculosis. These signatures were also distinct from those in control individuals. The signature of latent tuberculosis, which showed an over-representation of immune cytotoxic gene expression in whole blood, may help to determine protective immune factors against M. tuberculosis infection, since these patients are infected but most do not develop overt disease. This distinct transcriptional biomarker signature from active and latent TB patients may be also used to diagnose infection, and to monitor response to treatment with anti-mycobacterial drugs. In addition the signature in active tuberculosis patients will help to determine factors involved in immunopathogenesis and possibly lead to strategies for immune therapeutic intervention. This invention relates to a previous application that claimed the use of blood transcriptional biomarkers for the diagnosis of infections. However, this previous application did not disclose the existence of biomarkers for active and latent tuberculosis and focused rather on children with other acute infections (Ramillo, Blood, 2007).

The present identification of a transcriptional signature in blood from latent versus active TB patients can be used to test for patients with suspected Mycobacterium tuberculosis infection as well as for health screening/early detection of the disease. The invention also permits the evaluation of the response to treatment with anti-mycobacterial drugs. In this context, a test would also be particularly valuable in the context of drug trials, and particularly to assess drug treatments in Multi-Drug Resistant patients. Furthermore, the present invention may be used to obtain immediate, intermediate and long term data from the immune signature of latent tuberculosis to better define a protective immune response during vaccination trials. Also, the signature in active tuberculosis patients will help to determine factors involved in immunopathogenesis and possibly lead to strategies for immune therapeutic intervention.

The immune response to M. tuberculosis is complex and multifactorial. Although it is known that T cells and cytokines, such as TNF, IFN-γ and IL-12, are important for immune control of M. tuberculosis¹⁴⁻¹⁷ there remains an incomplete understanding of the host factors determining protection or pathogenesis¹⁶. Blood transcriptional profiling has been successfully applied to inflammatory diseases to improve diagnosis and the understanding of disease pathogenesis^{18,19}. However, the size and complexity of the data generated makes interpretation difficult, often forcing scientists to focus on a handful of candidate genes for further study²⁰, which may not be sufficient as specific biomarkers for diagnosis, and provide little information with respect to disease pathogenesis. Using independent and complementary bioinformatics techniques we have defined a transcriptional signature for active TB patients, which has driven further immunological analysis. Our comprehensive unbiased survey provides important insights into the immunopathogenesis of this complex disease, an improved understanding of which will aid advances in TB control.

A distinct whole blood transcriptional signature of active tuberculosis.

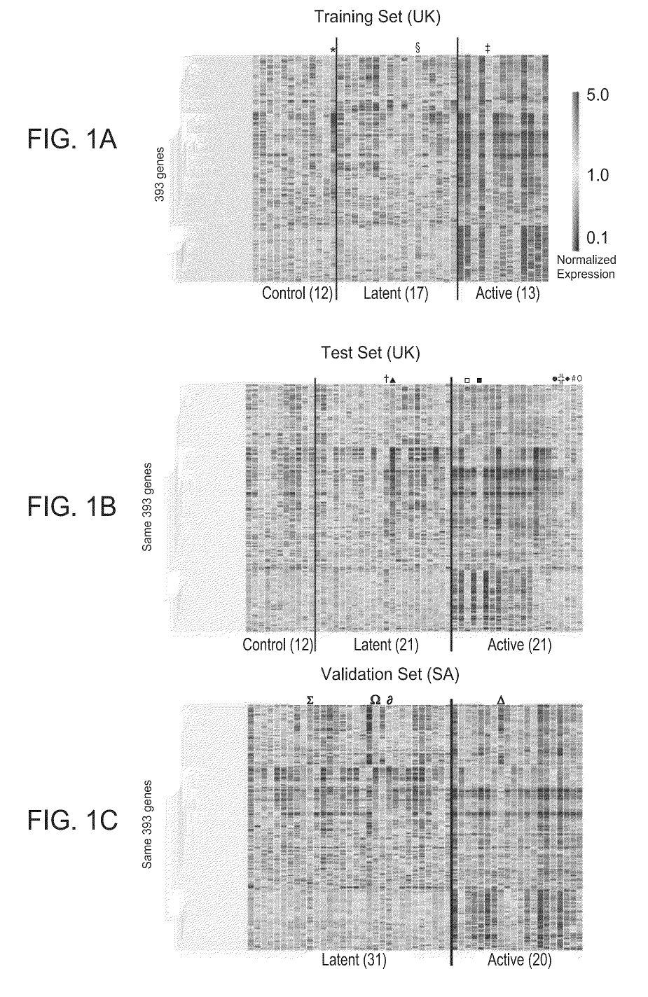

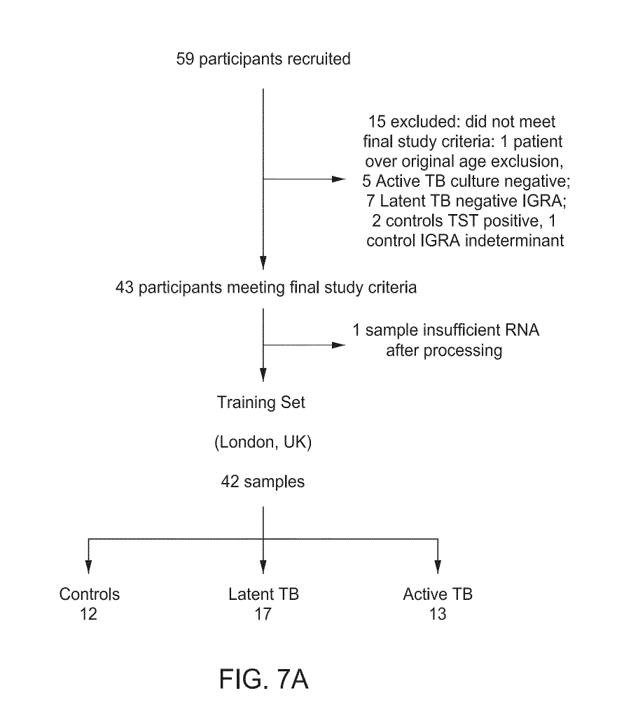

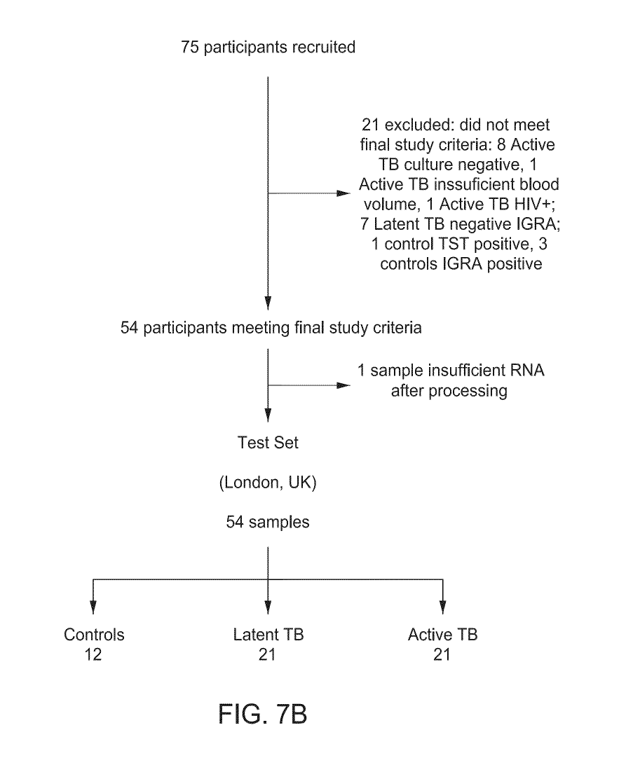

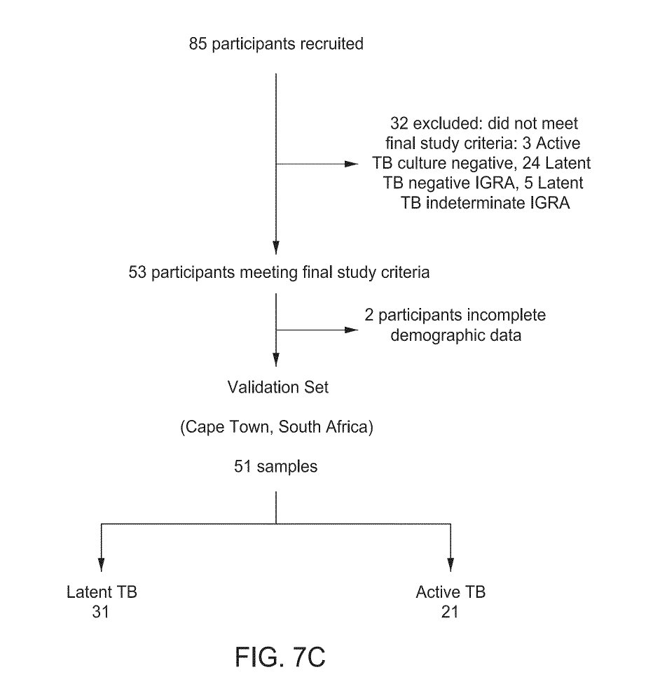

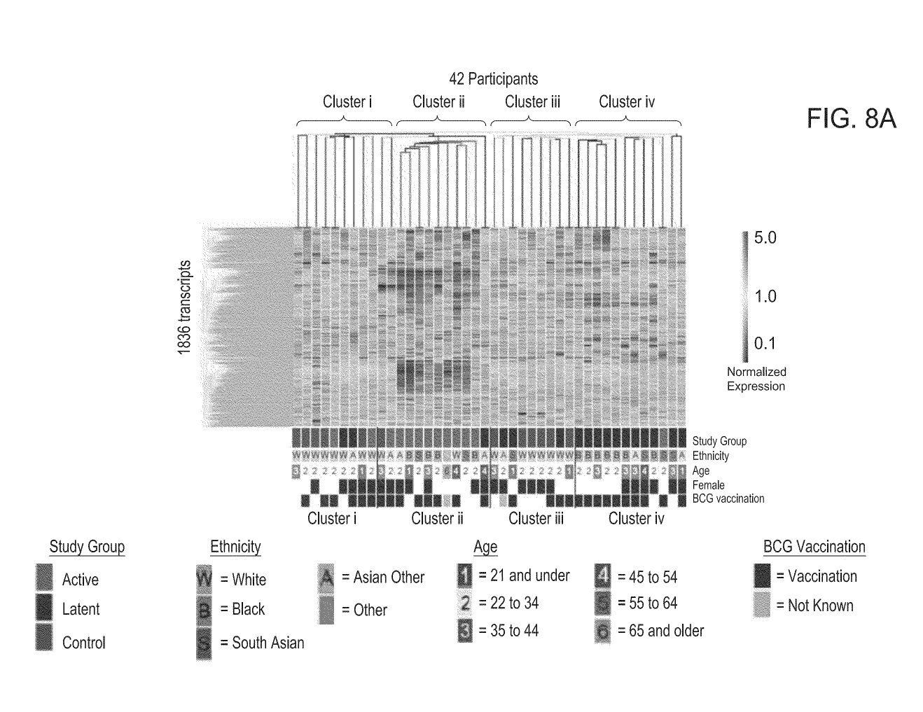

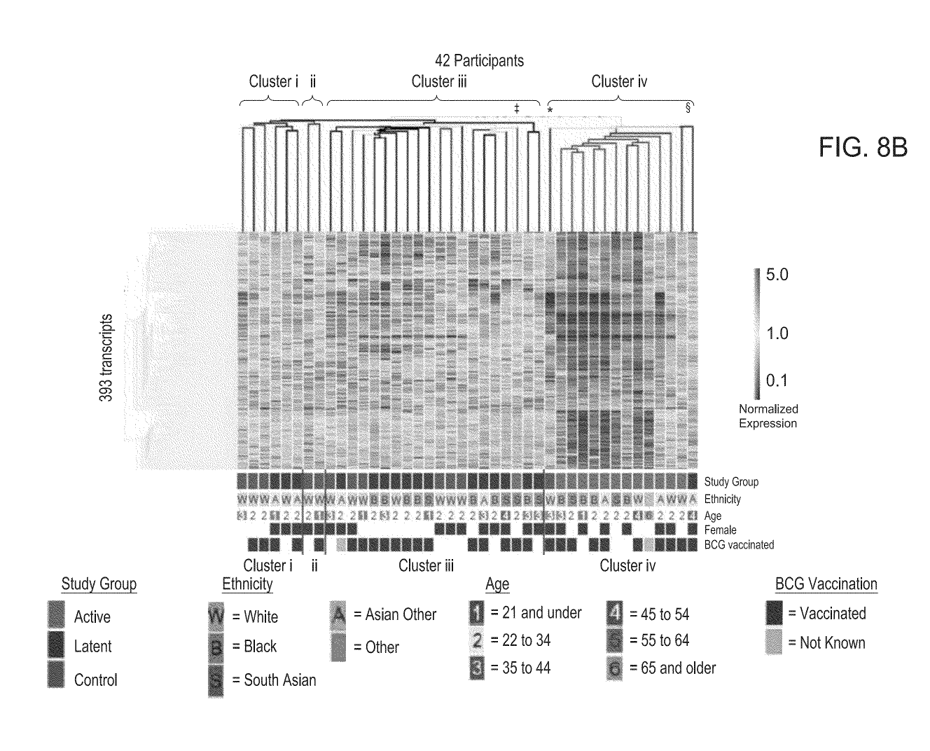

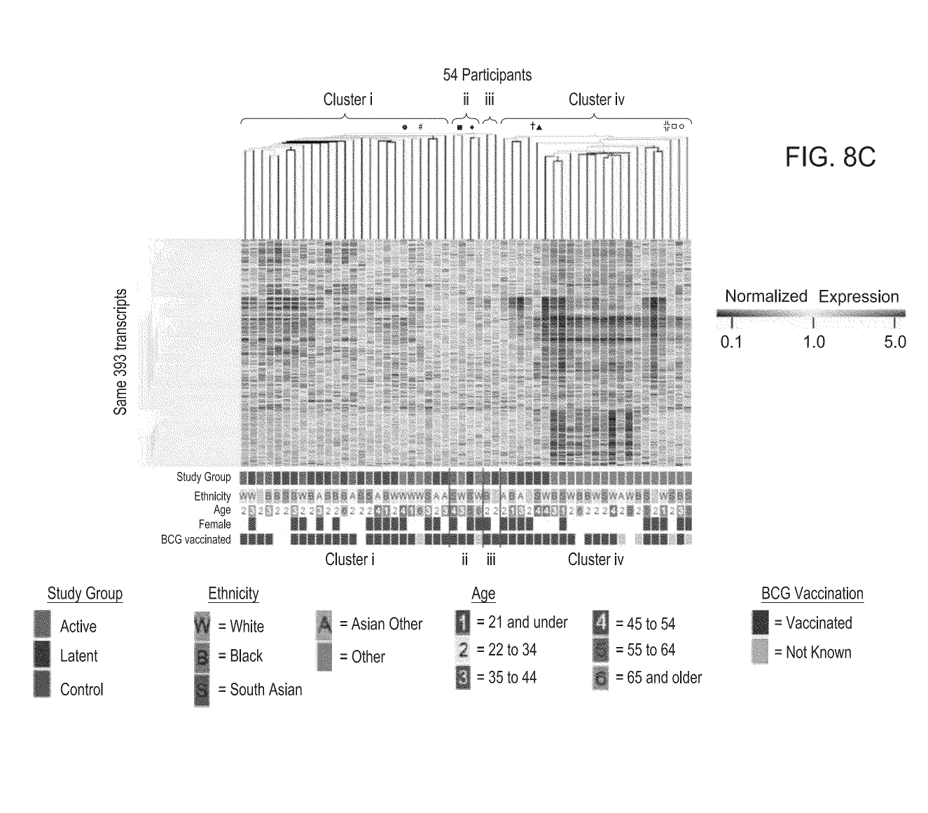

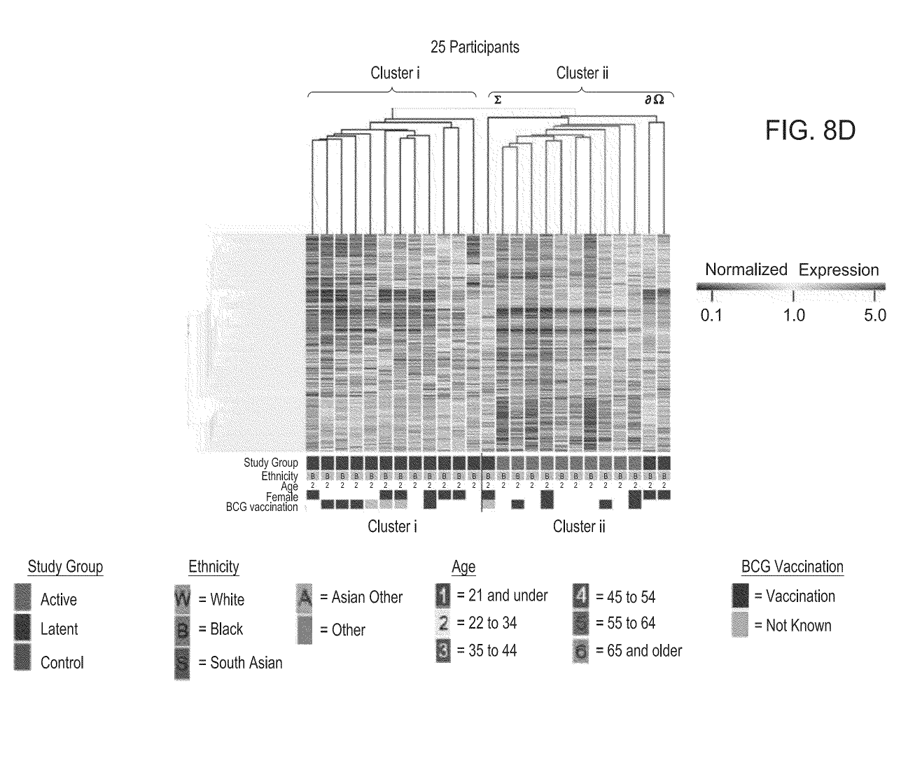

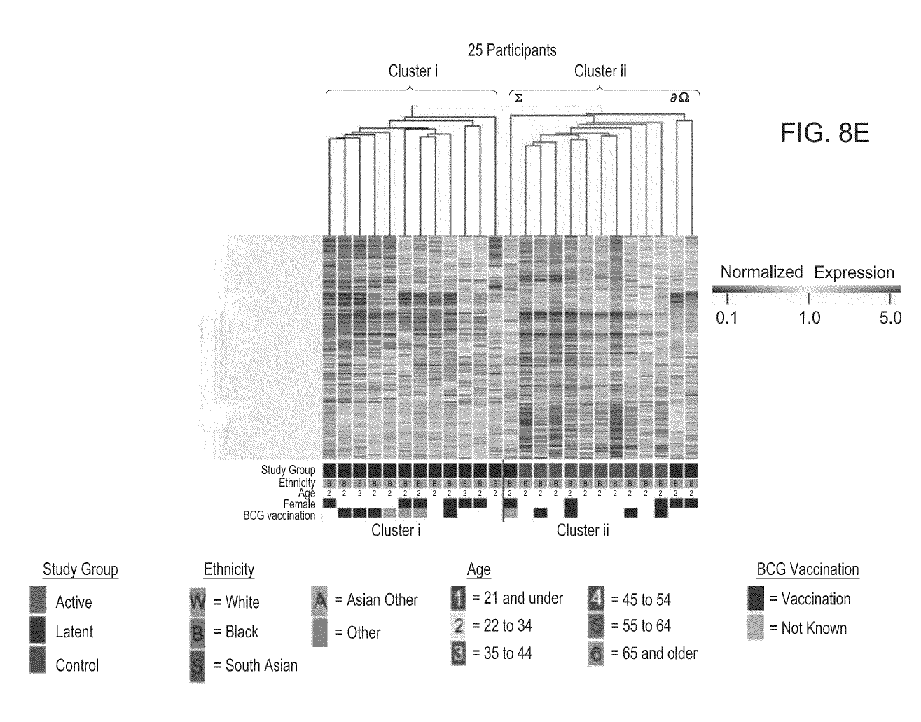

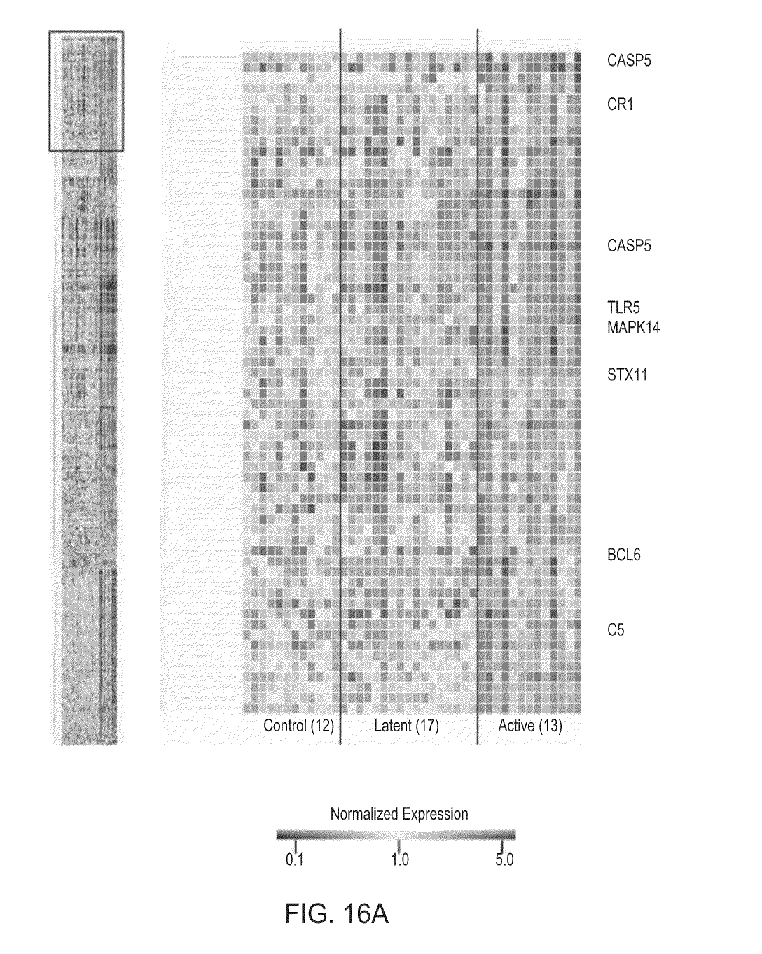

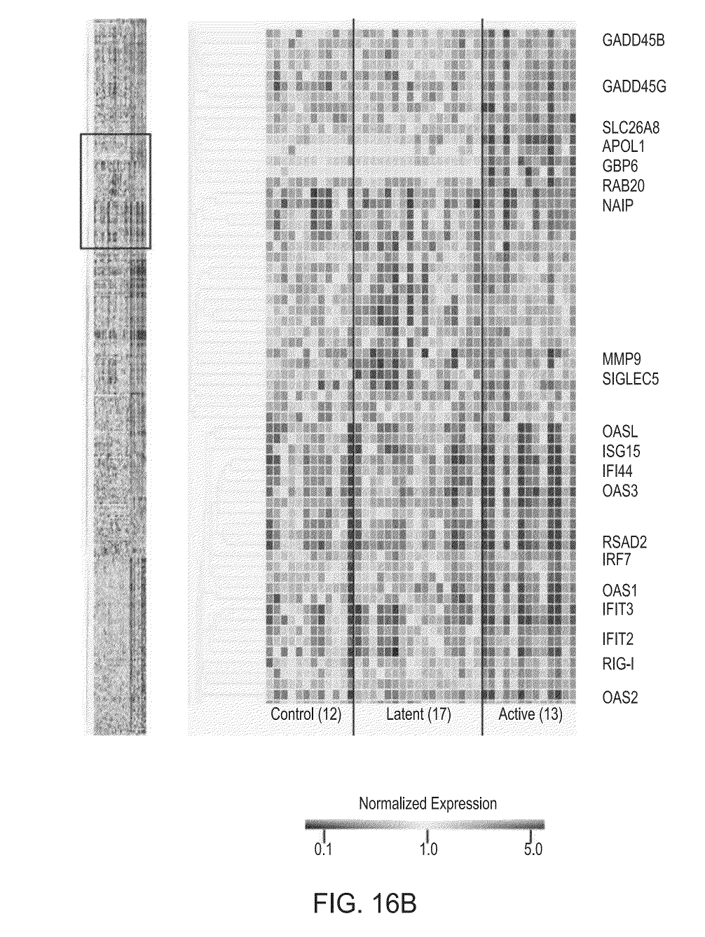

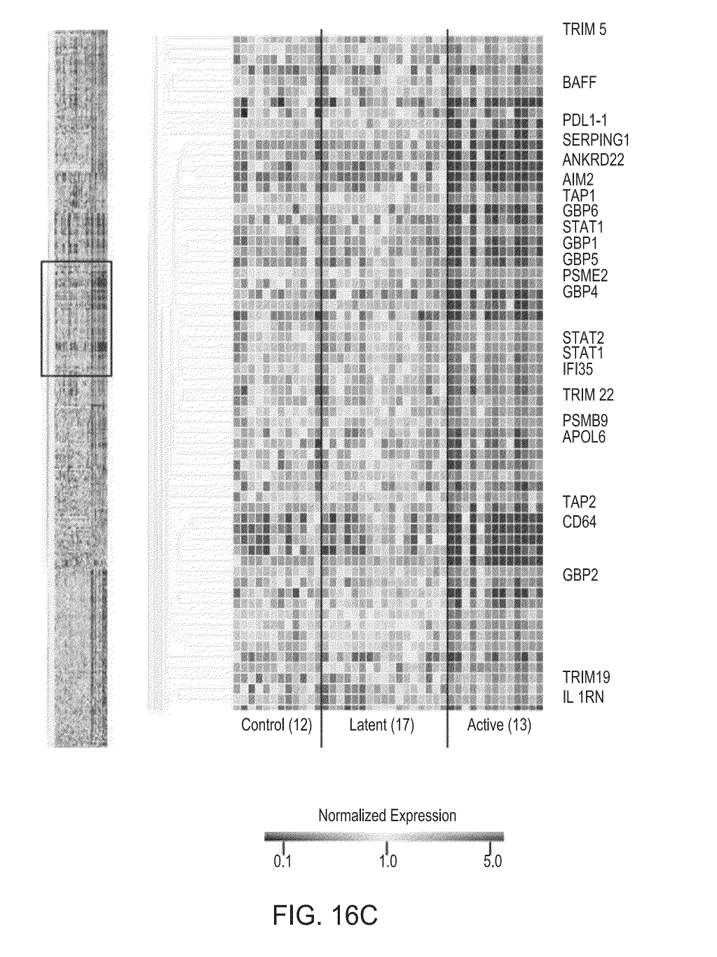

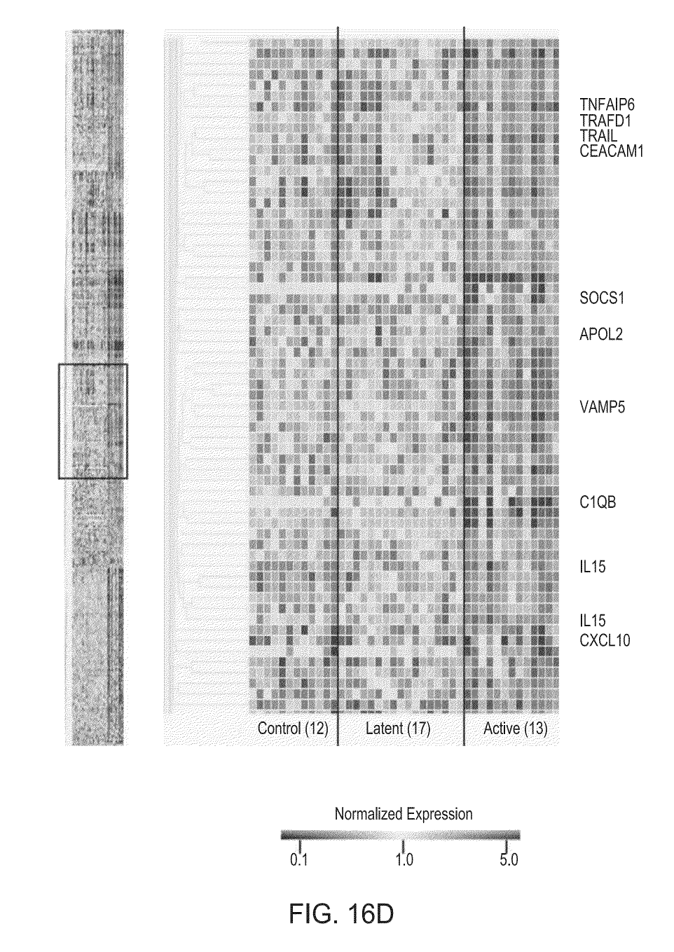

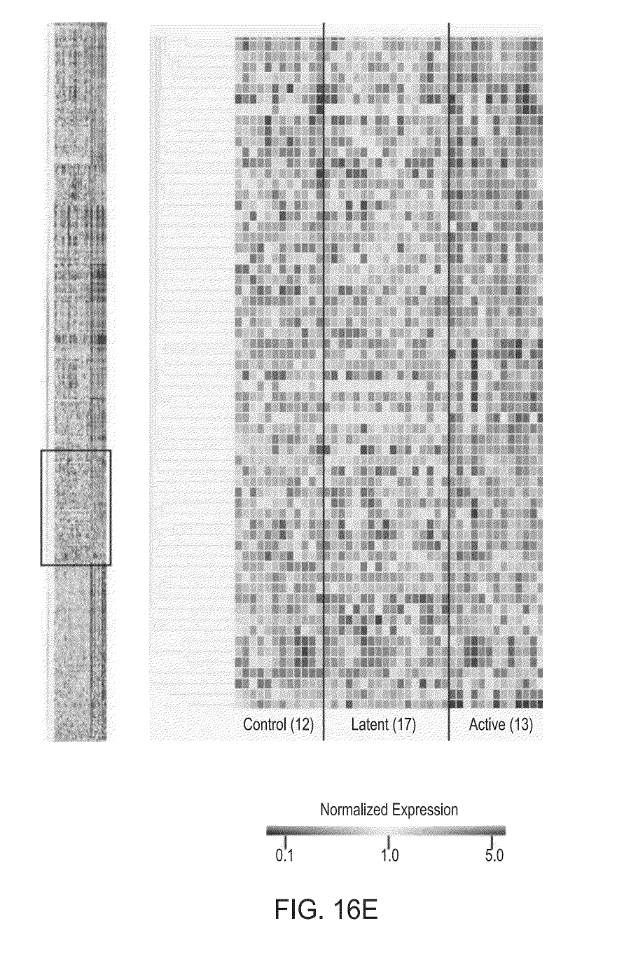

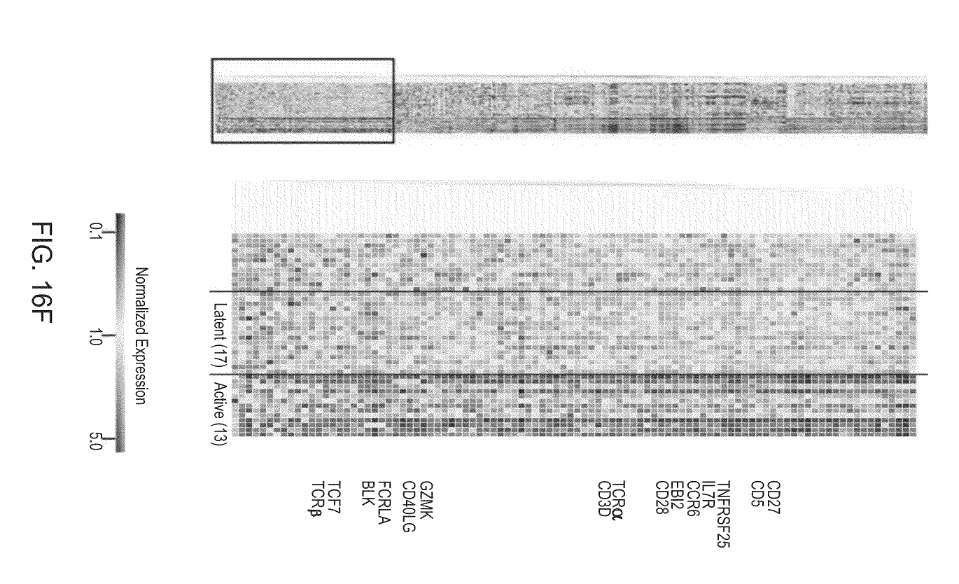

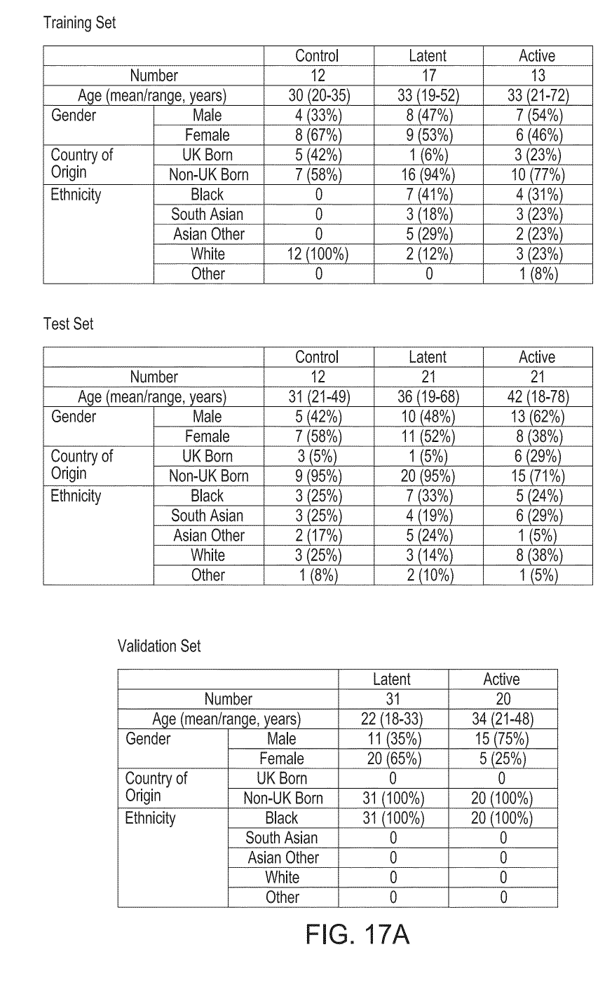

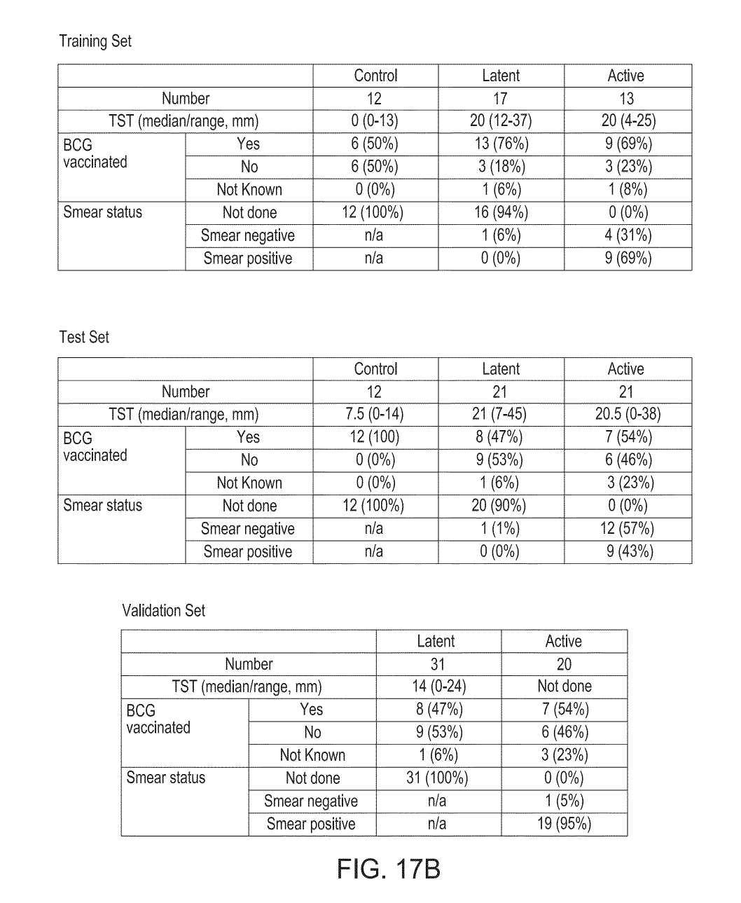

To obtain an unbiased comprehensive survey of host responses to M. tuberculosis infection, genome-wide transcriptional profiles from the blood of active TB patients, latent TB patients and healthy controls were generated using Illumina HT12 beadarrays. All patients were sampled before treatment. The diagnosis of active TB was confirmed by positive culture for M. tuberculosis. Latent TB patients were asymptomatic household contacts of active TB patients or new entrants from endemic countries, defined by a positive tuberculin-skin test (TST) (London) and a positive IGRA (London and South Africa). Healthy controls were recruited in London and were negative for all the above criteria. Three cohorts were independently recruited and sampled: a Training Set (recruited in London, January-September, 2007; 13 patients with active pulmonary TB; 17 patients with latent TB; and 12 healthy controls); a Test Set (recruited in London, October 2007-February 2009; 21 active TB patients; 21 latent TB patients; 12 healthy controls); and a Validation Set (recruited in a high burden, endemic region, Khayelitsha township near Cape Town, South Africa, (SA), May 2008-February, 2009; 20 active TB patients; 31 latent TB patients) (FIGS. 16 and 17; FIG. 7). Similarly, all processing and analysis of samples from the three cohorts were performed independently. The Training Set was used for knowledge discovery and an assessment of sample size adequacy. RNA was extracted from whole blood samples and processed as described in Methods. Resulting data were filtered to remove transcripts that were not detected (α=0.01) and had less than two-fold deviation in normalized expression from the median of all samples in greater than 10% of the samples constituting the dataset. This unsupervised filtering yielded a list of 1836 transcripts, which revealed a distinct signature within the active TB group, (FIG. 8a). This 1836 transcript list was then used to identify signature genes that were significantly differentially expressed among groups (Kruskal-Wallis ANOVA, with the false discovery rate equal to 0.01 using the Benjamini-Hochberg multiple testing correction). This yielded a list of 393 transcripts, which were subjected to hierarchical clustering by Pearson correlation with average linkage as the measure of distance between two clusters, creating a gene tree of transcripts with similar relative abundance. This is shown as a dendrogram, at the left of the heatmap, organizing the data from each individual into a unique transcriptional profile, shown grouped on the basis of clinical diagnosis (FIG. 1a). This revealed a distinct signature for active TB, which was absent in the majority of samples from latent TB patients or healthy controls.

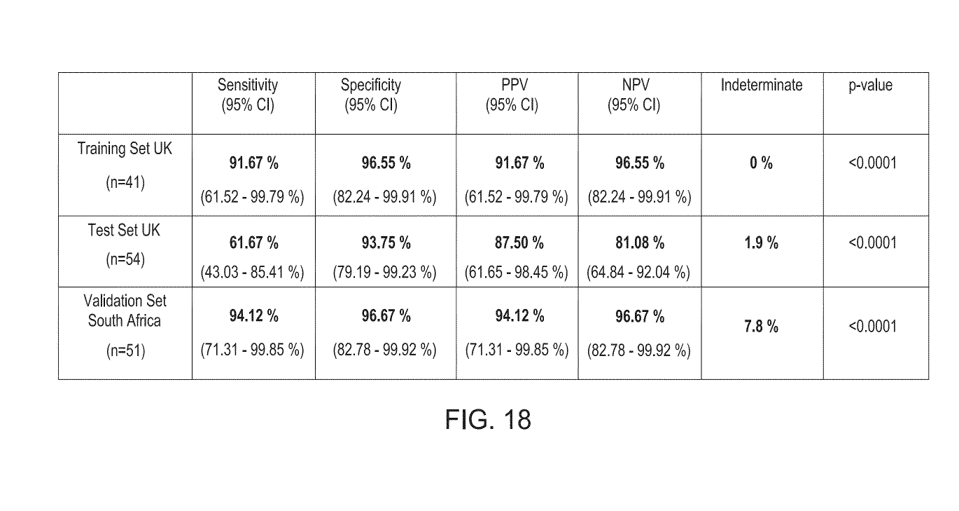

Having identified a putative transcriptional signature for active TB, it was important to confirm these findings in an independent cohort of patients. Microarray analyses are vulnerable to methodological, technical and statistical variability²¹⁻²³. Additionally it is likely that TB represents a diverse range of immune responses to M. tuberculosis infection, most likely influenced by ethnicity, geographical area, coinfection, age, and socioeconomic status^{11,13}. Thus, to ensure that our findings would be broadly applicable, we confirmed them in two additional independent cohorts, recruited at a later time. Samples from these two independent cohorts, the Test Set (London) and the Validation Set (South Africa) were processed and data were normalized as for the Training Set. As the aim of these additional validations was to independently confirm the signature defined in the Training Set, no filtering or selection of transcripts was performed. Rather, the pre-selected 393 transcript list and gene tree defined by analysis of the Training Set data were applied to the data obtained from the independent Test Set and Validation Set (SA). Hierarchical clustering algorithms were applied to the Test Set and Validation Set (SA) 393-transcript profiles, using Spearman correlation and average linkage as a measure of distance between clusters, to group together individual gene expression profiles according to their similarity, creating a “condition tree”, displayed along the upper edge of the heatmap (FIGS. 1b and 1c). This unsupervised hierarchical clustering of both the Test Set and Validation Set (SA) patient transcriptional profiles clearly show that active TB patients cluster independently of latent TB and healthy controls (FIG. 1b, London) or of latent TB (FIG. 1c; South Africa), with a significant association between cluster and study group (Pearson Chi-Squared Test p<0.0005) (FIGS. 1b and 1c), but not with ethnicity, age and gender (FIGS. 8b, 8c and 8d). However, the transcriptional profile of a small number of latent TB patients (approximately 10%-2/21 Test Set, London; 3/31 Validation Set (SA)), clustered together with that of the active TB patients (Marked † and ▴ in the Test Set, FIG. 1b; and marked τ, Ω and ∂ in the South Africa Validation Set FIG. 1c). We then tested the ability of the 393 transcript list to correctly classify Test Set and Validation Set samples as active TB or not (healthy or latent), without knowledge of the clinical diagnosis, using a class prediction tool based on the K-nearest neighbours class prediction method. The prediction model made 44 correct predictions, 9 incorrect predictions and made no prediction for 1 sample in the Test Set. This equated to a sensitivity of 61.67%, a specificity of 93.75%, and an indeterminate rate of 1.9%. The incorrect predictions in the Test Set, comprised the 5 latent TB patients classified as active TB indicated in the clustering analysis above; and 4 active TB patients predicted as not active TB. In the South African Validation Set there were 45 correct predictions, 2 incorrect (1 active, 1 latent) and no prediction for 4 samples. This gave a sensitivity of 94.12% and a specificity of 96.67%, but an indeterminate rate of 7.8% (FIG. 18).

TABLE 2
List of 393 Genes.

| | | | | Entrez | |
|---|---|---|---|---|---|
| Symbol | Probe | P-value | GI | Gene ID | Definition |
| | ILMN_1897745 | 0.00969 | 13708245 | | RST5526 Athersys RAGE Library Homo |
| | | | | | sapiens cDNA, mRNA sequence |
| NAIP | ILMN_2260082 | 0.00968 | 119393877 | 4671 | Homo sapiens NLR family, apoptosis inhibitory |
| | | | | | protein (NAIP), transcript variant 1, mRNA. |
| AGMAT | ILMN_1707169 | 0.00951 | 37537721 | 79814 | Homo sapiens agmatine ureohydrolase |
| | | | | | (agmatinase) (AGMAT), mRNA. |
| CD40LG | ILMN_1659077 | 0.00948 | 58331233 | 959 | Homo sapiens CD40 ligand (TNF superfamily, |
| | | | | | member 5, hyper-IgM syndrome) (CD40LG), |
| | | | | | mRNA. |
| PRDM1 | ILMN_2298159 | 0.00939 | 33946272 | 639 | Homo sapiens PR domain containing 1, with |
| | | | | | ZNF domain (PRDM1), transcript variant 1, |
| | | | | | mRNA. |
| LOC730092 | ILMN_1910120 | 0.00937 | 129270094 | | Homo sapiens RRN3 RNA polymerase I |
| | | | | | transcription factor homolog (S. cerevisiae) |
| | | | | | pseudogene (LOC730092) on chromosome 16. |
| FAM102A | ILMN_2401779 | 0.00937 | 78191786 | 399665 | Homo sapiens family with sequence similarity |
| | | | | | 102, member A (FAM102A), transcript variant |
| | | | | | 1, mRNA. |
| KRT72 | ILMN_1695812 | 0.00937 | 28372502 | 140807 | Homo sapiens keratin 72 (KRT72), mRNA. |
| KIAA0748 | ILMN_1690139 | 0.00933 | 89035529 | 9840 | PREDICTED: Homo sapiens KIAA0748 gene |
| | | | | | product, transcript variant 2 (KIAA0748), |
| | | | | | mRNA. |
| MORC2 | ILMN_2103591 | 0.00927 | 7662339 | 22880 | Homo sapiens MORC family CW-type zinc |
| | | | | | finger 2 (MORC2), mRNA. |
| OASL | ILMN_1681721 | 0.00918 | 38016933 | 8638 | Homo sapiens 2′-5′-oligoadenylate synthetase- |
| | | | | | like (OASL), transcript variant 1, mRNA. |
| CD151 | ILMN_1661589 | 0.00915 | 87159821 | 977 | Homo sapiens CD151 molecule (Raph blood |
| | | | | | group) (CD151), transcript variant 4, mRNA. |
| CR1 | ILMN_2388112 | 0.00902 | 86793035 | 1378 | Homo sapiens complement component (3b/4b) |
| | | | | | receptor 1 (Knops blood group) (CR1), |
| | | | | | transcript variant F, mRNA. |
| SPOCK2 | ILMN_1656287 | 0.00884 | 7662035 | 9806 | Homo sapiens sparc/osteonectin, cwcv and |
| | | | | | kazal-like domains proteoglycan (testican) 2 |
| | | | | | (SPOCK2), mRNA. |
| SOCS3 | ILMN_1781001 | 0.00884 | 45439351 | 9021 | Homo sapiens suppressor of cytokine signaling |
| | | | | | 3 (SOCS3), mRNA. |
| DHRS9 | ILMN_1727150 | 0.00865 | 40548396 | 10170 | Homo sapiens dehydrogenase/reductase (SDR |
| | | | | | family) member 9 (DHRS9), transcript variant |
| | | | | | 2, mRNA. |
| P2RY14 | ILMN_2342835 | 0.00842 | 125625351 | 9934 | Homo sapiens purinergic receptor P2Y, G- |
| | | | | | protein coupled, 14 (P2RY14), transcript |
| | | | | | variant 2, mRNA. |
| BCAS4 | ILMN_2325506 | 0.00836 | 58294159 | 55653 | Homo sapiens breast carcinoma amplified |
| | | | | | sequence 4 (BCAS4), transcript variant 1, |
| | | | | | mRNA. |
| MGC22014 | ILMN_1796832 | 0.00829 | 88953265 | 200424 | PREDICTED: Homo sapiens hypothetical |
| | | | | | protein MGC22014 (MGC22014), mRNA. |
| RHBDF2 | ILMN_1735792 | 0.00829 | 93352557 | 79651 | Homo sapiens rhomboid 5 homolog 2 |
| | | | | | (Drosophila) (RHBDF2), transcript variant 2, |
| | | | | | mRNA. |
| SOCS1 | ILMN_1774733 | 0.00829 | 4507232 | 8651 | Homo sapiens suppressor of cytokine signaling |
| | | | | | 1 (SOCS1), mRNA. |
| ETS1 | ILMN_2122103 | 0.00829 | 41393580 | 2113 | Homo sapiens v-ets erythroblastosis virus E26 |
| | | | | | oncogene homolog 1 (avian) (ETS1), mRNA. |
| KIAA1026 | ILMN_1770927 | 0.00826 | 66864888 | 23254 | Homo sapiens kazrin (KIAA1026), transcript |
| | | | | | variant B, mRNA. |
| | ILMN_1868912 | 0.00826 | 22477381 | | Homo sapiens T cell receptor beta variable 21- |
| | | | | | 1, mRNA (cDNA clone MGC: 46491 |
| | | | | | IMAGE: 5225843), complete cds |
| TLR2 | ILMN_1772387 | 0.00826 | 68160956 | 7097 | Homo sapiens toll-like receptor 2 (TLR2), |
| | | | | | mRNA. |
| LBH | ILMN_1660794 | 0.00821 | 113413661 | 81606 | PREDICTED: Homo sapiens hypothetical |
| | | | | | protein DKFZp566J091 (LBH), mRNA. |
| TPM2 | ILMN_1789196 | 0.00821 | 47519615 | 7169 | Homo sapiens tropomyosin 2 (beta) (TPM2), |
| | | | | | transcript variant 2, mRNA. |
| TPD52 | ILMN_2381064 | 0.00805 | 70608192 | 7163 | Homo sapiens tumor protein D52 (TPD52), |
| | | | | | transcript variant 3, mRNA. |
| FCRLA | ILMN_1691071 | 0.00801 | 42544162 | 84824 | Homo sapiens Fc receptor-like A (FCRLA), |
| | | | | | mRNA. |
| HLA-DPB1 | ILMN_1749070 | 0.00795 | 24797075 | 3115 | Homo sapiens major histocompatibility |
| | | | | | complex, class II, DP beta 1 (HLA-DPB1), |
| | | | | | mRNA. |
| ABCG1 | ILMN_2329927 | 0.00795 | 46592897 | 9619 | Homo sapiens ATP-binding cassette, sub- |
| | | | | | family G (WHITE), member 1 (ABCG1), |
| | | | | | transcript variant 2, mRNA. |
| NAT6 | ILMN_1765001 | 0.00793 | 46048438 | 24142 | Homo sapiens N-acetyltransferase 6 (NAT6), |
| | | | | | mRNA. |
| CLUAP1 | ILMN_1750596 | 0.00785 | 13435144 | 23059 | Homo sapiens clusterin associated protein 1 |
| | | | | | (CLUAP1), transcript variant 2, mRNA. |
| PASK | ILMN_1754858 | 0.00784 | 35038527 | 23178 | Homo sapiens PAS domain containing |
| | | | | | serine/threonine kinase (PASK), mRNA. |
| ATP6V0E2 | ILMN_1785095 | 0.00775 | 154689665 | 155066 | Homo sapiens ATPase, H+ transporting V0 |
| | | | | | subunit e2 (ATP6V0E2), transcript variant 1, |
| | | | | | mRNA. |
| POLR1E | ILMN_1678934 | 0.00775 | 11968046 | 64425 | Homo sapiens polymerase (RNA) I polypeptide |
| | | | | | E, 53 kDa (POLR1E), mRNA. |
| MGC42367 | ILMN_1776121 | 0.00765 | 46409355 | 343990 | Homo sapiens similar to 2010300C02Rik |
| | | | | | protein (MGC42367), mRNA. |
| HNRPA1L-2 | ILMN_2220283 | 0.00763 | 115529279 | | Homo sapiens heterogeneous nuclear |
| | | | | | ribonucleoprotein A1 pseudogene (HNRPA1L- |
| | | | | | 2) on chromosome 19. |
| NAIP | ILMN_1760189 | 0.00762 | 119393877 | 4671 | Homo sapiens NLR family, apoptosis inhibitory |
| | | | | | protein (NAIP), transcript variant 1, mRNA. |
| ALDH1A1 | ILMN_2096372 | 0.00762 | 25777722 | 216 | Homo sapiens aldehyde dehydrogenase 1 |
| | | | | | family, member A1 (ALDH1A1), mRNA. |
| ID3 | ILMN_1732296 | 0.00753 | 32171181 | 3399 | Homo sapiens inhibitor of DNA binding 3, |
| | | | | | dominant negative helix-loop-helix protein |
| | | | | | (ID3), mRNA. |
| ZNF429 | ILMN_1695413 | 0.00748 | 116256454 | 353088 | Homo sapiens zinc finger protein 429 |
| | | | | | (ZNF429), mRNA. |
| SNORD13 | ILMN_1892403 | 0.00747 | 94721317 | | Homo sapiens small nucleolar RNA, C/D box |
| | | | | | 13 (SNORD13) on chromosome 8. |
| CD38 | ILMN_2233783 | 0.00747 | 38454325 | 952 | Homo sapiens CD38 molecule (CD38), mRNA. |
| C16orf30 | ILMN_1751559 | 0.00724 | 112807181 | 79652 | Homo sapiens chromosome 16 open reading |
| | | | | | frame 30 (C16orf30), mRNA. |
| CXCL6 | ILMN_1779234 | 0.00723 | 52851409 | 6372 | Homo sapiens chemokine (C—X—C motif) ligand |
| | | | | | 6 (granulocyte chemotactic protein 2) |
| | | | | | (CXCL6), mRNA. |
| HK2 | ILMN_1723486 | 0.00723 | 40806188 | 3099 | Homo sapiens hexokinase 2 (HK2), mRNA. |
| CLEC4D | ILMN_1808979 | 0.00722 | 37577120 | 338339 | Homo sapiens C-type lectin domain family 4, |
| | | | | | member D (CLEC4D), mRNA. |
| SLC30A1 | ILMN_2067852 | 0.00722 | 52352802 | 7779 | Homo sapiens solute carrier family 30 (zinc |
| | | | | | transporter), member 1 (SLC30A1), mRNA. |
| TNFRSF25 | ILMN_2299661 | 0.00722 | 89142744 | 8718 | Homo sapiens tumor necrosis factor receptor |
| | | | | | superfamily, member 25 (TNFRSF25), |
| | | | | | transcript variant 12, mRNA. |
| OAS2 | ILMN_1709333 | 0.00718 | 74229018 | 4939 | Homo sapiens 2′-5′-oligoadenylate synthetase 2, |
| | | | | | 69/71 kDa (OAS2), transcript variant 1, mRNA. |
| ASGR2 | ILMN_1694966 | 0.00718 | 18426876 | 433 | Homo sapiens asialoglycoprotein receptor 2 |
| | | | | | (ASGR2), transcript variant 3, mRNA. |
| MAGEE1 | ILMN_2205032 | 0.00712 | 20143481 | 57692 | Homo sapiens melanoma antigen family E, 1 |
| | | | | | (MAGEE1), mRNA. |
| LOC642606 | ILMN_1664597 | 0.00701 | 89035480 | 642606 | PREDICTED: Homo sapiens hypothetical |
| | | | | | protein LOC642606 (LOC642606), mRNA. |
| KIAA1641 | ILMN_1699521 | 0.00673 | 88956579 | 57730 | PREDICTED: Homo sapiens KIAA1641, |
| | | | | | transcript variant 7 (KIAA1641), mRNA. |
| MEF2D | ILMN_1763228 | 0.0067 | 40254821 | 4209 | Homo sapiens myocyte enhancer factor 2D |
| | | | | | (MEF2D), mRNA. |
| LOC650795 | ILMN_1790771 | 0.00661 | 89037605 | 650795 | PREDICTED: Homo sapiens similar to T-cell |
| | | | | | receptor alpha chain V region PY14 precursor |
| | | | | | (LOC650795), mRNA. |
| BMX | ILMN_1672307 | 0.00654 | 42544181 | 660 | Homo sapiens BMX non-receptor tyrosine |
| | | | | | kinase (BMX), mRNA. |
| CXCL10 | ILMN_1791759 | 0.00646 | 149999381 | 3627 | Homo sapiens chemokine (C—X—C motif) ligand |
| | | | | | 10 (CXCL10), mRNA. |
| KCNJ15 | ILMN_1659770 | 0.00646 | 25777637 | 3772 | Homo sapiens potassium inwardly-rectifying |
| | | | | | channel, subfamily J, member 15 (KCNJ15), |
| | | | | | transcript variant 1, mRNA. |
| LBH | ILMN_1811507 | 0.00641 | 113413661 | 81606 | PREDICTED: Homo sapiens hypothetical |
| | | | | | protein DKFZp566J091 (LBH), mRNA. |
| PASK | ILMN_1667022 | 0.00641 | 35038527 | 23178 | Homo sapiens PAS domain containing |
| | | | | | serine/threonine kinase (PASK), mRNA. |
| EVI2A | ILMN_1662747 | 0.00625 | 51511748 | 2123 | Homo sapiens ecotropic viral integration site |
| | | | | | 2A (EVI2A), transcript variant 1, mRNA. |
| LIN7A | ILMN_1806293 | 0.00621 | 49574521 | 8825 | Homo sapiens lin-7 homolog A (C. elegans) |
| | | | | | (LIN7A), mRNA. |
| ETV7 | ILMN_1700671 | 0.00619 | 31542589 | 51513 | Homo sapiens ets variant gene 7 (TEL2 |
| | | | | | oncogene) (ETV7), mRNA. |
| CLEC12A | ILMN_2403228 | 0.00614 | 94557289 | 160364 | Homo sapiens C-type lectin domain family 12, |
| | | | | | member A (CLEC12A), transcript variant 1, |
| | | | | | mRNA. |
| P2RY14 | ILMN_2258409 | 0.00606 | 125625351 | 9934 | Homo sapiens purinergic receptor P2Y, G- |
| | | | | | protein coupled, 14 (P2RY14), transcript |
| | | | | | variant 2, mRNA. |
| TXNDC3 | ILMN_1691334 | 0.00606 | 148839371 | 51314 | Homo sapiens thioredoxin domain containing 3 |
| | | | | | (spermatozoa) (TXNDC3), mRNA. |
| NDRG2 | ILMN_2361603 | 0.00596 | 42544219 | 57447 | Homo sapiens NDRG family member 2 |
| | | | | | (NDRG2), transcript variant 6, mRNA. |
| CECR6 | ILMN_1702229 | 0.00592 | 54607075 | 27439 | Homo sapiens cat eye syndrome chromosome |
| | | | | | region, candidate 6 (CECR6), mRNA. |
| | ILMN_1915188 | 0.00586 | 34529437 | | Homo sapiens cDNA FLJ41813 fis, clone |
| | | | | | NT2RI2011450 |
| DDX58 | ILMN_1797001 | 0.00576 | 77732514 | 23586 | Homo sapiens DEAD (Asp-Glu-Ala-Asp) box |
| | | | | | polypeptide 58 (DDX58), mRNA. |
| TIMM10 | ILMN_1765332 | 0.0057 | 93004075 | 26519 | Homo sapiens translocase of inner |
| | | | | | mitochondrial membrane 10 homolog (yeast) |
| | | | | | (TIMM10), nuclear gene encoding |
| | | | | | mitochondrial protein, mRNA. |
| MYC | ILMN_2110908 | 0.00569 | 71774082 | 4609 | Homo sapiens v-myc myelocytomatosis viral |
| | | | | | oncogene homolog (avian) (MYC), mRNA. |
| SOD2 | ILMN_2406501 | 0.00569 | 67782308 | 6648 | Homo sapiens superoxide dismutase 2, |
| | | | | | mitochondrial (SOD2), nuclear gene encoding |
| | | | | | mitochondrial protein, transcript variant 3, |
| | | | | | mRNA. |
| ISG15 | ILMN_2054019 | 0.00569 | 4826773 | 9636 | Homo sapiens ISG15 ubiquitin-like modifier |
| | | | | | (ISG15), mRNA. |
| TXNDC12 | ILMN_1783753 | 0.00569 | 23943808 | 51060 | Homo sapiens thioredoxin domain containing |
| | | | | | 12 (endoplasmic reticulum) (TXNDC12), |
| | | | | | mRNA. |
| IFI44L | ILMN_1723912 | 0.00568 | 5803026 | 10964 | Homo sapiens interferon-induced protein 44- |
| | | | | | like (IFI44L), mRNA. |
| BMX | ILMN_1796138 | 0.00568 | 42544180 | 660 | Homo sapiens BMX non-receptor tyrosine |
| | | | | | kinase (BMX), mRNA. |
| CDK5RAP2 | ILMN_2415529 | 0.00568 | 58535452 | 55755 | Homo sapiens CDK5 regulatory subunit |
| | | | | | associated protein 2 (CDK5RAP2), transcript |
| | | | | | variant 2, mRNA. |
| | ILMN_1823172 | 0.00566 | 32217345 | | EST10086 human nasopharynx Homo sapiens |
| | | | | | cDNA, mRNA sequence |
| FER1L3 | ILMN_2370976 | 0.00564 | 19718757 | 26509 | Homo sapiens fer-1-like 3, myoferlin (C. elegans) |
| | | | | | (FER1L3), transcript variant 1, |
| | | | | | mRNA. |
| IFIT5 | ILMN_1696654 | 0.0056 | 6912629 | 24138 | Homo sapiens interferon-induced protein with |
| | | | | | tetratricopeptide repeats 5 (IFIT5), mRNA. |
| KCNJ15 | ILMN_2396903 | 0.00558 | 25777639 | 3772 | Homo sapiens potassium inwardly-rectifying |
| | | | | | channel, subfamily J, member 15 (KCNJ15), |
| | | | | | transcript variant 3, mRNA. |
| ZAK | ILMN_1698803 | 0.00549 | 82880647 | 51776 | Homo sapiens sterile alpha motif and leucine |
| | | | | | zipper containing kinase AZK (ZAK), |
| | | | | | transcript variant 1, mRNA. |
| | ILMN_1844464 | 0.00545 | 36748 | | Human mRNA for T-cell specific protein |
| ATP8B2 | ILMN_1782057 | 0.0054 | 56121819 | 57198 | Homo sapiens ATPase, class I, type 8B, |
| | | | | | member 2 (ATP8B2), transcript variant 1, |
| | | | | | mRNA. |
| XAF1 | ILMN_2370573 | 0.0054 | 40288192 | 54739 | Homo sapiens XIAP associated factor 1 |
| | | | | | (XAF1), transcript variant 2, mRNA. |
| C5 | ILMN_1746819 | 0.00527 | 38016946 | 727 | Homo sapiens complement component 5 (C5), |
| | | | | | mRNA. |
| GAS6 | ILMN_1779558 | 0.00511 | 4557616 | 2621 | Homo sapiens growth arrest-specific 6 (GAS6), |
| | | | | | mRNA. |
| PIK3IP1 | ILMN_1719986 | 0.00499 | 51317357 | 113791 | Homo sapiens phosphoinositide-3-kinase |
| | | | | | interacting protein 1 (PIK3IP1), mRNA. |
| SIPA1L2 | ILMN_1732923 | 0.00499 | 112421012 | 57568 | Homo sapiens signal-induced proliferation- |
| | | | | | associated 1 like 2 (SIPA1L2), mRNA. |
| ANXA3 | ILMN_1694548 | 0.00498 | 96304463 | 306 | Homo sapiens annexin A3 (ANXA3), mRNA. |
| HIST2H2BF | ILMN_1670093 | 0.00493 | 84992988 | 440689 | Homo sapiens histone cluster 2, H2bf |
| | | | | | (HIST2H2BF), mRNA. |
| CR1 | ILMN_1742601 | 0.00486 | 86793108 | 1378 | Homo sapiens complement component (3b/4b) |
| | | | | | receptor 1 (Knops blood group) (CR1), |
| | | | | | transcript variant S, mRNA. |
| ABLIM1 | ILMN_1785424 | 0.00461 | 51173716 | 3983 | Homo sapiens actin binding LIM protein 1 |
| | | | | | (ABLIM1), transcript variant 4, mRNA. |
| IKZF3 | ILMN_2300695 | 0.00461 | 38045957 | 22806 | Homo sapiens IKAROS family zinc finger 3 |
| | | | | | (Aiolos) (IKZF3), transcript variant 1, mRNA. |
| FAM26F | ILMN_2066849 | 0.00461 | 62988335 | 441168 | Homo sapiens family with sequence similarity |
| | | | | | 26, member F (FAM26F), mRNA. |
| CAPN12 | ILMN_1787514 | 0.0046 | 46852396 | 147968 | Homo sapiens calpain 12 (CAPN12), mRNA. |
| CLEC12A | ILMN_2292178 | 0.00458 | 94557289 | 160364 | Homo sapiens C-type lectin domain family 12, |
| | | | | | member A (CLEC12A), transcript variant 1, |
| | | | | | mRNA. |
| CDK5RAP2 | ILMN_1655990 | 0.00455 | 58535450 | 55755 | Homo sapiens CDK5 regulatory subunit |
| | | | | | associated protein 2 (CDK5RAP2), transcript |
| | | | | | variant 1, mRNA. |
| QPCT | ILMN_1741727 | 0.00454 | 68216098 | 25797 | Homo sapiens glutaminyl-peptide |
| | | | | | cyclotransferase (glutaminyl cyclase) (QPCT), |
| | | | | | mRNA. |
| | ILMN_1873034 | 0.00444 | 47682415 | | Homo sapiens T cell receptor alpha locus, |
| | | | | | mRNA (cDNA clone MGC: 88342 |
| | | | | | IMAGE: 30352166), complete cds |
| SERPINA1 | ILMN_2256050 | 0.00444 | 50363218 | 5265 | Homo sapiens serpin peptidase inhibitor, clade |
| | | | | | A (alpha-1 antiproteinase, antitrypsin), member |
| | | | | | 1 (SERPINA1), transcript variant 2, mRNA. |
| GAS6 | ILMN_1784749 | 0.00434 | 4557616 | 2621 | Homo sapiens growth arrest-specific 6 (GAS6), |
| | | | | | mRNA. |
| GADD45G | ILMN_1651498 | 0.00434 | 9790905 | 10912 | Homo sapiens growth arrest and DNA-damage- |
| | | | | | inducible, gamma (GADD45G), mRNA. |
| TMEM51 | ILMN_1674985 | 0.00434 | 8922276 | 55092 | Homo sapiens transmembrane protein 51 |
| | | | | | (TMEM51), mRNA. |
| CD274 | ILMN_1701914 | 0.0043 | 20070268 | 29126 | Homo sapiens CD274 molecule (CD274), |
| | | | | | mRNA. |
| TSHZ2 | ILMN_1655611 | 0.0042 | 153945733 | 128553 | Homo sapiens teashirt zinc finger homeobox 2 |
| | | | | | (TSHZ2), mRNA. |
| LILRA5 | ILMN_1726545 | 0.0042 | 32895360 | 353514 | Homo sapiens leukocyte immunoglobulin-like |
| | | | | | receptor, subfamily A (with TM domain), |
| | | | | | member 5 (LILRA5), transcript variant 3, |
| | | | | | mRNA. |
| CD3D | ILMN_2325837 | 0.00411 | 98985800 | 915 | Homo sapiens CD3d molecule, delta (CD3- |
| | | | | | TCR complex) (CD3D), transcript variant 2, |
| | | | | | mRNA. |
| KIAA1026 | ILMN_1798458 | 0.00403 | 66864888 | 23254 | Homo sapiens kazrin (KIAA1026), transcript |
| | | | | | variant B, mRNA. |
| B3GNT8 | ILMN_1741389 | 0.00399 | 42821106 | 374907 | Homo sapiens UDP-GlcNAc:betaGal beta-1,3- |
| | | | | | N-acetylglucosaminyltransferase 8 (B3GNT8), |
| | | | | | mRNA. |
| NR3C2 | ILMN_2210934 | 0.00399 | 4505198 | 4306 | Homo sapiens nuclear receptor subfamily 3, |
| | | | | | group C, member 2 (NR3C2), mRNA. |
| HERC5 | ILMN_1729749 | 0.00398 | 110825981 | 51191 | Homo sapiens hect domain and RLD 5 |
| | | | | | (HERC5), mRNA. |
| OAS3 | ILMN_1745397 | 0.00398 | 45007006 | 4940 | Homo sapiens 2′-5′-oligoadenylate synthetase 3, |
| | | | | | 100 kDa (OAS3), mRNA. |
| IL18RAP | ILMN_1721762 | 0.00397 | 27477087 | 8807 | Homo sapiens interleukin 18 receptor accessory |
| | | | | | protein (IL18RAP), mRNA. |
| LOC653610 | ILMN_1695435 | 0.00394 | 88943486 | 653610 | PREDICTED: Homo sapiens similar to Histone |
| | | | | | H2A.o (H2A/o) (H2A.2) (H2a-615) |
| | | | | | (LOC653610), mRNA. |
| GPR109A | ILMN_1750497 | 0.00393 | 41152145 | 338442 | Homo sapiens G protein-coupled receptor 109A |
| | | | | | (GPR109A), mRNA. |
| LOC728519 | ILMN_1679620 | 0.00393 | 113416624 | 728519 | PREDICTED: Homo sapiens similar to |
| | | | | | Baculoviral IAP repeat-containing protein 1 |
| | | | | | (Neuronal apoptosis inhibitory protein) |
| | | | | | (LOC728519), mRNA. |
| TRIM5 | ILMN_1737599 | 0.00393 | 15011943 | 85363 | Homo sapiens tripartite motif-containing 5 |
| | | | | | (TRIM5), transcript variant gamma, mRNA. |
| LOC642161 | ILMN_1651403 | 0.00393 | 89026482 | 642161 | PREDICTED: Homo sapiens similar to T-cell |
| | | | | | receptor beta chain V region CTL-L17 |
| | | | | | precursor (LOC642161), mRNA. |
| TNFRSF25 | ILMN_1765109 | 0.00393 | 23200036 | 8718 | Homo sapiens tumor necrosis factor receptor |
| | | | | | superfamily, member 25 (TNFRSF25), |
| | | | | | transcript variant 10, mRNA. |
| IFI6 | ILMN_2347798 | 0.00393 | 94538329 | 2537 | Homo sapiens interferon, alpha-inducible |
| | | | | | protein 6 (IFI6), transcript variant 2, mRNA. |
| TCN2 | ILMN_1740572 | 0.00392 | 21071009 | 6948 | Homo sapiens transcobalamin II; macrocytic |
| | | | | | anemia (TCN2), mRNA. |
| C11orf1 | ILMN_2128967 | 0.0038 | 118766341 | 64776 | Homo sapiens chromosome 11 open reading |
| | | | | | frame 1 (C11orf1), mRNA. |
| IGF2BP3 | ILMN_1807423 | 0.00374 | 30795211 | 10643 | Homo sapiens insulin-like growth factor 2 |
| | | | | | mRNA binding protein 3 (IGF2BP3), mRNA. |
| LOC728014 | ILMN_1711699 | 0.00373 | 113423526 | 728014 | PREDICTED: Homo sapiens similar to |
| | | | | | huntingtin interacting protein 1 related |
| | | | | | (LOC728014), mRNA. |
| LTB4R | ILMN_1747251 | 0.00366 | 31881791 | 1241 | Homo sapiens leukotriene B4 receptor |
| | | | | | (LTB4R), mRNA. |
| LOC648984 | ILMN_1801254 | 0.00366 | 89065840 | 648984 | PREDICTED: Homo sapiens similar to |
| | | | | | Baculoviral IAP repeat-containing protein 1 |
| | | | | | (Neuronal apoptosis inhibitory protein) |
| | | | | | (LOC648984), mRNA. |
| DHRS12 | ILMN_1669177 | 0.00366 | 13375996 | 79758 | Homo sapiens dehydrogenase/reductase (SDR |
| | | | | | family) member 12 (DHRS12), transcript |
| | | | | | variant 2, mRNA. |
| | ILMN_1887868 | 0.00358 | 7019830 | | Homo sapiens cDNA FLJ20012 fis, clone |
| | | | | | ADKA03438 |
| ADAM7 | ILMN_1750294 | 0.00353 | 114326452 | 8756 | Homo sapiens ADAM metallopeptidase domain |
| | | | | | 7 (ADAM7), mRNA. |
| BIN1 | ILMN_1674160 | 0.00352 | 21536406 | 274 | Homo sapiens bridging integrator 1 (BIN1), |
| | | | | | transcript variant 4, mRNA. |
| TCF7 | ILMN_2367141 | 0.00352 | 42518077 | 6932 | Homo sapiens transcription factor 7 (T-cell |
| | | | | | specific, HMG-box) (TCF7), transcript variant |
| | | | | | 2, mRNA. |
| SLC22A4 | ILMN_1685057 | 0.00352 | 24497489 | 6583 | Homo sapiens solute carrier family 22 (organic |
| | | | | | cation/ergothioneine transporter), member 4 |
| | | | | | (SLC22A4), mRNA. |
| XRN1 | ILMN_2384216 | 0.00349 | 110624786 | 54464 | Homo sapiens 5′-3′ exoribonuclease 1 (XRN1), |
| | | | | | transcript variant 2, mRNA. |
| DKFZp761E198 | ILMN_1717594 | 0.00344 | 149999370 | 91056 | Homo sapiens DKFZp761E198 protein |
| | | | | | (DKFZp761E198), mRNA. |
| C1QB | ILMN_1796409 | 0.00342 | 87298827 | 713 | Homo sapiens complement component 1, q |
| | | | | | subcomponent, B chain (C1QB), mRNA. |
| LIMK2 | ILMN_1687960 | 0.00332 | 73390131 | 3985 | Homo sapiens LIM domain kinase 2 (LIMK2), |
| | | | | | transcript variant 2b, mRNA. |
| LOC653867 | ILMN_1678633 | 0.0033 | 88986878 | 653867 | PREDICTED: Homo sapiens similar to |
| | | | | | Occludin (LOC653867), mRNA. |
| IRF7 | ILMN_1798181 | 0.0033 | 98985817 | 3665 | Homo sapiens interferon regulatory factor 7 |
| | | | | | (IRF7), transcript variant b, mRNA. |
| MMP9 | ILMN_1796316 | 0.00326 | 74272286 | 4318 | Homo sapiens matrix metallopeptidase 9 |
| | | | | | (gelatinase B, 92 kDa gelatinase, 92 kDa type IV |
| | | | | | collagenase) (MMP9), mRNA. |
| SMARCD3 | ILMN_2309180 | 0.00323 | 51477705 | 6604 | Homo sapiens SWI/SNF related, matrix |
| | | | | | associated, actin dependent regulator of |
| | | | | | chromatin, subfamily d, member 3 |
| | | | | | (SMARCD3), transcript variant 2, mRNA. |
| KLF12 | ILMN_1762801 | 0.00322 | 115392135 | 11278 | Homo sapiens Kruppel-like factor 12 (KLF12), |
| | | | | | mRNA. |
| DKFZp761P0423 | ILMN_1757872 | 0.00322 | 89027874 | 157285 | PREDICTED: Homo sapiens hypothetical |
| | | | | | protein DKFZp761P0423 (DKFZp761P0423), |
| | | | | | mRNA. |
| PVRIG | ILMN_1688279 | 0.00315 | 57863284 | 79037 | Homo sapiens poliovirus receptor related |
| | | | | | immunoglobulin domain containing (PVRIG), |
| | | | | | mRNA. |
| SOX8 | ILMN_1789244 | 0.00315 | 30179902 | 30812 | Homo sapiens SRY (sex determining region |
| | | | | | Y)-box 8 (SOX8), mRNA. |
| CLYBL | ILMN_1663538 | 0.00315 | 45545436 | 171425 | Homo sapiens citrate lyase beta like (CLYBL), |
| | | | | | mRNA. |
| ENTPD1 | ILMN_1773125 | 0.00311 | 147905699 | 953 | Homo sapiens ectonucleoside triphosphate |
| | | | | | diphosphohydrolase 1 (ENTPD1), transcript |
| | | | | | variant 2, mRNA. |
| RSAD2 | ILMN_1657871 | 0.0031 | 90186265 | 91543 | Homo sapiens radical S-adenosyl methionine |
| | | | | | domain containing 2 (RSAD2), mRNA. |
| PARP10 | ILMN_1710844 | 0.0031 | 113420558 | 84875 | PREDICTED: Homo sapiens poly (ADP- |
| | | | | | ribose) polymerase family, member 10 |
| | | | | | (PARP10), mRNA. |
| CD27 | ILMN_1688959 | 0.00309 | 117422442 | 939 | Homo sapiens CD27 molecule (CD27), mRNA. |
| ABHD14A | ILMN_1794213 | 0.00302 | 34147328 | 25864 | Homo sapiens abhydrolase domain containing |
| | | | | | 14A (ABHD14A), mRNA. |
| OAS1 | ILMN_1675640 | 0.00302 | 74229014 | 4938 | Homo sapiens 2′,5′-oligoadenylate synthetase 1, |
| | | | | | 40/46 kDa (OAS1), transcript variant 3, mRNA. |
| SATB1 | ILMN_1690646 | 0.00302 | 33356175 | 6304 | Homo sapiens SATB homeobox 1 (SATB1), |
| | | | | | mRNA. |
| PLSCR1 | ILMN_1745242 | 0.00302 | 10863876 | 5359 | Homo sapiens phospholipid scramblase 1 |
| | | | | | (PLSCR1), mRNA. |
| | ILMN_1889841 | 0.00299 | 27825332 | | BX092531 NCI_CGAP_Kid5 Homo sapiens |
| | | | | | cDNA clone IMAGp998I114659; |
| | | | | | IMAGE: 1900882, mRNA sequence |
| PGLYRP1 | ILMN_1704870 | 0.00295 | 4827035 | 8993 | Homo sapiens peptidoglycan recognition |
| | | | | | protein 1 (PGLYRP1), mRNA. |
| LBH | ILMN_2315979 | 0.00295 | 13569871 | 81606 | Homo sapiens limb bud and heart development |
| | | | | | homolog (mouse) (LBH), mRNA. |
| CLEC12A | ILMN_1663142 | 0.00294 | 94557292 | 160364 | Homo sapiens C-type lectin domain family 12, |
| | | | | | member A (CLEC12A), transcript variant 2, |
| | | | | | mRNA. |
| DHRS12 | ILMN_1719915 | 0.00293 | 13375996 | 79758 | Homo sapiens dehydrogenase/reductase (SDR |
| | | | | | family) member 12 (DHRS12), transcript |
| | | | | | variant 2, mRNA. |
| LIMK2 | ILMN_1660624 | 0.00291 | 73390139 | 3985 | Homo sapiens LIM domain kinase 2 (LIMK2), |
| | | | | | transcript variant 1, mRNA. |
| KREMEN1 | ILMN_1772697 | 0.00288 | 89191857 | 83999 | Homo sapiens kringle containing |
| | | | | | transmembrane protein 1 (KREMEN1), |
| | | | | | transcript variant 4, mRNA. |
| FCGBP | ILMN_2302757 | 0.00285 | 4503680 | 8857 | Homo sapiens Fc fragment of IgG binding |
| | | | | | protein (FCGBP), mRNA. |
| PARP9 | ILMN_2053527 | 0.00285 | 13899296 | 83666 | Homo sapiens poly (ADP-ribose) polymerase |
| | | | | | family, member 9 (PARP9), mRNA. |
| C9orf66 | ILMN_1717248 | 0.00285 | 22749172 | 157983 | Homo sapiens chromosome 9 open reading |
| | | | | | frame 66 (C9orf66), mRNA. |
| CD59 | ILMN_1724789 | 0.00284 | 42716300 | 966 | Homo sapiens CD59 molecule, complement |
| | | | | | regulatory protein (CD59), transcript variant 2, |
| | | | | | mRNA. |
| EPB41L3 | ILMN_2109197 | 0.00284 | 32490571 | 23136 | Homo sapiens erythrocyte membrane protein |
| | | | | | band 4.1-like 3 (EPB41L3), mRNA. |
| CMPK2 | ILMN_1783621 | 0.00284 | 117606369 | 129607 | Homo sapiens cytidine monophosphate (UMP- |
| | | | | | CMP) kinase 2, mitochondrial (CMPK2), |
| | | | | | nuclear gene encoding mitochondrial protein, |
| | | | | | mRNA. |
| BCL6 | ILMN_1746053 | 0.00284 | 21040335 | 604 | Homo sapiens B-cell CLL/lymphoma 6 (zinc |
| | | | | | finger protein 51) (BCL6), transcript variant 2, |
| | | | | | mRNA. |
| LOC648099 | ILMN_1672687 | 0.00284 | 89065616 | 648099 | PREDICTED: Homo sapiens similar to positive |
| | | | | | cofactor 2, glutamine/Q-rich-associated protein |
| | | | | | isoform b (LOC648099), mRNA. |
| C11orf82 | ILMN_1790100 | 0.00284 | 25072198 | 220042 | Homo sapiens chromosome 11 open reading |
| | | | | | frame 82 (C11orf82), mRNA. |
| CASP5 | ILMN_1722158 | 0.00283 | 4757913 | 838 | Homo sapiens caspase 5, apoptosis-related |
| | | | | | cysteine peptidase (CASP5), mRNA. |
| CCR6 | ILMN_1690907 | 0.00282 | 150417990 | 1235 | Homo sapiens chemokine (C-C motif) receptor |
| | | | | | 6 (CCR6), transcript variant 2, mRNA. |
| CACNA1E | ILMN_1664047 | 0.00281 | 53832004 | 777 | Homo sapiens calcium channel, voltage- |
| | | | | | dependent, R type, alpha 1E subunit |
| | | | | | (CACNA1E), mRNA. |
| DHRS9 | ILMN_2281502 | 0.00281 | 40548399 | 10170 | Homo sapiens dehydrogenase/reductase (SDR |
| | | | | | family) member 9 (DHRS9), transcript variant |
| | | | | | 1, mRNA. |
| TNFSF13B | ILMN_1758418 | 0.00281 | 23510443 | 10673 | Homo sapiens tumor necrosis factor (ligand) |
| | | | | | superfamily, member 13b (TNFSF13B), |
| | | | | | mRNA. |
| FCAR | ILMN_2365091 | 0.00278 | 19743872 | 2204 | Homo sapiens Fc fragment of IgA, receptor for |
| | | | | | (FCAR), transcript variant 10, mRNA. |
| C19orf59 | ILMN_1762713 | 0.00274 | 109698610 | 199675 | Homo sapiens chromosome 19 open reading |
| | | | | | frame 59 (C19orf59), mRNA. |
| GPR109B | ILMN_1677693 | 0.00264 | 5174460 | 8843 | Homo sapiens G protein-coupled receptor 109B |
| | | | | | (GPR109B), mRNA. |
| FAIM3 | ILMN_1775542 | 0.00264 | 34147517 | 9214 | Homo sapiens Fas apoptotic inhibitory |
| | | | | | molecule 3 (FAIM3), mRNA. |
| | ILMN_1886655 | 0.00264 | 50477326 | | full-length cDNA clone CS0DI056YK21 of |
| | | | | | Placenta Cot 25-normalized of Homo sapiens |
| | | | | | (human) |
| CD5 | ILMN_1753112 | 0.00264 | 24431962 | 921 | Homo sapiens CD5 molecule (CD5), mRNA. |
| SRPK1 | ILMN_1798804 | 0.00264 | 47419935 | 6732 | Homo sapiens SFRS protein kinase 1 (SRPK1), |
| | | | | | mRNA. |
| LOC552891 | ILMN_1767809 | 0.00252 | 21361096 | 552891 | Homo sapiens hypothetical protein LOC552891 |
| | | | | | (LOC552891), mRNA. |
| IL15 | ILMN_2369221 | 0.0025 | 26787983 | 3600 | Homo sapiens interleukin 15 (IL15), transcript |
| | | | | | variant 1, mRNA. |
| IFITM1 | ILMN_1801246 | 0.00249 | 150010588 | 8519 | Homo sapiens interferon induced |
| | | | | | transmembrane protein 1 (9-27) (IFITM1), |
| | | | | | mRNA. |
| ASGR2 | ILMN_2342638 | 0.00249 | 18426876 | 433 | Homo sapiens asialoglycoprotein receptor 2 |
| | | | | | (ASGR2), transcript variant 3, mRNA. |
| | ILMN_1835092 | 0.00245 | 21176493 | | AGENCOURT_7914287 NIH_MGC_71 Homo |
| | | | | | sapiens cDNA clone IMAGE: 6156595 5, |
| | | | | | mRNA sequence |
| GPR141 | ILMN_2092333 | 0.00245 | 32401434 | 353345 | Homo sapiens G protein-coupled receptor 141 |
| | | | | | (GPR141), mRNA. |
| NOV | ILMN_1787186 | 0.00245 | 19923725 | 4856 | Homo sapiens nephroblastoma overexpressed |
| | | | | | gene (NOV), mRNA. |
| PML | ILMN_1728019 | 0.00245 | 89039089 | 5371 | PREDICTED: Homo sapiens promyelocytic |
| | | | | | leukemia, transcript variant 12 (PML), mRNA. |
| CREB5 | ILMN_1731714 | 0.00245 | 59938769 | 9586 | Homo sapiens cAMP responsive element |
| | | | | | binding protein 5 (CREB5), transcript variant 1, |
| | | | | | mRNA. |
| | ILMN_1860051 | 0.00245 | 1621766 | | HUMGS0004661 Human adult (K. Okubo) |
| | | | | | Homo sapiens cDNA 3, mRNA sequence |
| EPHA4 | ILMN_1672022 | 0.00239 | 45439363 | 2043 | Homo sapiens EPH receptor A4 (EPHA4), |
| | | | | | mRNA. |
| CDK5R1 | ILMN_1730928 | 0.00239 | 34304373 | 8851 | Homo sapiens cyclin-dependent kinase 5, |
| | | | | | regulatory subunit 1 (p35) (CDK5R1), mRNA. |
| LOC652755 | ILMN_1788237 | 0.00239 | 89077285 | 652755 | PREDICTED: Homo sapiens similar to |
| | | | | | Baculoviral IAP repeat-containing protein 1 |
| | | | | | (Neuronal apoptosis inhibitory protein) |
| | | | | | (LOC652755), mRNA. |
| ZBP1 | ILMN_1765994 | 0.00239 | 13540544 | 81030 | Homo sapiens Z-DNA binding protein 1 |
| | | | | | (ZBP1), mRNA. |
| LILRB4 | ILMN_2355953 | 0.00239 | 125987587 | 11006 | Homo sapiens leukocyte immunoglobulin-like |
| | | | | | receptor, subfamily B (with TM and ITIM |
| | | | | | domains), member 4 (LILRB4), transcript |
| | | | | | variant 2, mRNA. |
| URG4 | ILMN_1777811 | 0.00232 | 117968346 | 55665 | Homo sapiens up-regulated gene 4 (URG4), |
| | | | | | nuclear gene encoding mitochondrial protein, |
| | | | | | transcript variant 1, mRNA. |
| CACNA1I | ILMN_2300664 | 0.00231 | 51093858 | 8911 | Homo sapiens calcium channel, voltage- |
| | | | | | dependent, T type, alpha 1I subunit |
| | | | | | (CACNA1I), transcript variant 2, mRNA. |
| SELM | ILMN_1651429 | 0.00228 | 46370092 | 140606 | Homo sapiens selenoprotein M (SELM), |
| | | | | | mRNA. |
| OASL | ILMN_1674811 | 0.00228 | 38016929 | 8638 | Homo sapiens 2′-5′-oligoadenylate synthetase- |
| | | | | | like (OASL), transcript variant 2, mRNA. |
| COP1 | ILMN_1726591 | 0.00221 | 62953111 | 114769 | Homo sapiens caspase-1 dominant-negative |
| | | | | | inhibitor pseudo-ICE (COP1), transcript variant |
| | | | | | 2, mRNA. |
| FRMD3 | ILMN_1698725 | 0.00219 | 34222248 | 257019 | Homo sapiens FERM domain containing 3 |
| | | | | | (FRMD3), mRNA. |
| IL7R | ILMN_1691341 | 0.00217 | 88987627 | 3575 | PREDICTED: Homo sapiens interleukin 7 |
| | | | | | receptor (IL7R), mRNA. |
| C4orf18 | ILMN_1761941 | 0.00217 | 144445990 | 51313 | Homo sapiens chromosome 4 open reading |
| | | | | | frame 18 (C4orf18), transcript variant 2, |
| | | | | | mRNA. |
| GPR84 | ILMN_1785345 | 0.00208 | 9966838 | 53831 | Homo sapiens G protein-coupled receptor 84 |
| | | | | | (GPR84), mRNA. |
| ZNF525 | ILMN_1748432 | 0.00208 | 89056927 | 170958 | PREDICTED: Homo sapiens zinc finger |
| | | | | | protein 525 (ZNF525), mRNA. |
| EBI2 | ILMN_1798706 | 0.00208 | 50962860 | 1880 | Homo sapiens Epstein-Barr virus induced gene |
| | | | | | 2 (lymphocyte-specific G protein-coupled |
| | | | | | receptor) (EBI2), mRNA. |
| C12orf57 | ILMN_1812191 | 0.00206 | 34147536 | 113246 | Homo sapiens chromosome 12 open reading |
| | | | | | frame 57 (C12orf57), mRNA. |
| SLC26A8 | ILMN_1672575 | 0.00206 | 20336284 | 116369 | Homo sapiens solute carrier family 26, member |
| | | | | | 8 (SLC26A8), transcript variant 2, mRNA. |
| C9orf72 | ILMN_1762508 | 0.00206 | 37039614 | 203228 | Homo sapiens chromosome 9 open reading |
| | | | | | frame 72 (C9orf72), transcript variant 2, |
| | | | | | mRNA. |
| GRAP | ILMN_2264011 | 0.00206 | 50659102 | 10750 | Homo sapiens GRB2-related adaptor protein |
| | | | | | (GRAP), mRNA. |
| IFITM3 | ILMN_1805750 | 0.00206 | 148612841 | 10410 | Homo sapiens interferon induced |
| | | | | | transmembrane protein 3 (1-8U) (IFITM3), |
| | | | | | mRNA. |
| NELL2 | ILMN_1725417 | 0.00205 | 5453765 | 4753 | Homo sapiens NEL-like 2 (chicken) (NELL2), |
| | | | | | mRNA. |
| LPCAT2 | ILMN_1796335 | 0.00204 | 47106078 | 54947 | Homo sapiens lysophosphatidylcholine |
| | | | | | acyltransferase 2 (LPCAT2), mRNA. |
| BLK | ILMN_1668277 | 0.00203 | 33469981 | 640 | Homo sapiens B lymphoid tyrosine kinase |
| | | | | | (BLK), mRNA. |
| IFIT3 | ILMN_1701789 | 0.00201 | 72534657 | 3437 | Homo sapiens interferon-induced protein with |
| | | | | | tetratricopeptide repeats 3 (IFIT3), mRNA. |
| AGPAT3 | ILMN_1654010 | 0.00197 | 41327762 | 56894 | Homo sapiens 1-acylglycerol-3-phosphate O- |
| | | | | | acyltransferase 3 (AGPAT3), mRNA. |
| AFF1 | ILMN_1673119 | 0.00195 | 5174572 | 4299 | Homo sapiens AF4/FMR2 family, member 1 |
| | | | | | (AFF1), mRNA. |
| PFKFB3 | ILMN_2186061 | 0.00195 | 42476167 | 5209 | Homo sapiens 6-phosphofructo-2- |
| | | | | | kinase/fructose-2,6-biphosphatase 3 (PFKFB3), |
| | | | | | mRNA. |
| KLF12 | ILMN_1714444 | 0.00195 | 115392135 | 11278 | Homo sapiens Kruppel-like factor 12 (KLF12), |
| | | | | | mRNA. |
| IFI44 | ILMN_1760062 | 0.00193 | 141802167 | 10561 | Homo sapiens interferon-induced protein 44 |
| | | | | | (IFI44), mRNA. |
| NBN | ILMN_1734833 | 0.00184 | 67189763 | 4683 | Homo sapiens nibrin (NBN), transcript variant |
| | | | | | 1, mRNA. |
| SLC26A8 | ILMN_1656849 | 0.00179 | 20336283 | 116369 | Homo sapiens solute carrier family 26, member |
| | | | | | 8 (SLC26A8), transcript variant 1, mRNA. |
| OSM | ILMN_1780546 | 0.00179 | 28178862 | 5008 | Homo sapiens oncostatin M (OSM), mRNA. |
| SP140 | ILMN_2246882 | 0.00178 | 52487276 | 11262 | Homo sapiens SP140 nuclear body protein |
| | | | | | (SP140), transcript variant 2, mRNA. |
| KIF1B | ILMN_1743034 | 0.00173 | 41393558 | 23095 | Homo sapiens kinesin family member 1B |
| | | | | | (KIF1B), transcript variant 2, mRNA. |
| KLF12 | ILMN_1797375 | 0.0017 | 21071072 | 11278 | Homo sapiens Kruppel-like factor 12 (KLF12), |
| | | | | | transcript variant 2, mRNA. |
| TRIB2 | ILMN_1714700 | 0.0017 | 11056053 | 28951 | Homo sapiens tribbles homolog 2 (Drosophila) |
| | | | | | (TRIB2), mRNA. |
| SLC26A8 | ILMN_2394210 | 0.0017 | 20336284 | 116369 | Homo sapiens solute carrier family 26, member |
| | | | | | 8 (SLC26A8), transcript variant 2, mRNA. |
| GNG10 | ILMN_1757074 | 0.00166 | 89941472 | 2790 | Homo sapiens guanine nucleotide binding |
| | | | | | protein (G protein), gamma 10 (GNG10), |
| | | | | | mRNA. |
| OAS1 | ILMN_2410826 | 0.00166 | 74229014 | 4938 | Homo sapiens 2′,5′-oligoadenylate synthetase 1, |
| | | | | | 40/46 kDa (OAS1), transcript variant 3, mRNA. |
| | ILMN_1909770 | 0.00166 | 10437260 | | Homo sapiens cDNA: FLJ21199 fis, clone |
| | | | | | COL00235 |
| XAF1 | ILMN_1742618 | 0.00165 | 40288192 | 54739 | Homo sapiens XIAP associated factor 1 |
| | | | | | (XAF1), transcript variant 2, mRNA. |
| LOC650799 | ILMN_1715436 | 0.00165 | 89037607 | 650799 | PREDICTED: Homo sapiens similar to Ig |
| | | | | | lambda chain V-I region BL2 precursor |
| | | | | | (LOC650799), mRNA. |
| IL1RN | ILMN_1689734 | 0.00165 | 27894318 | 3557 | Homo sapiens interleukin 1 receptor antagonist |
| | | | | | (IL1RN), transcript variant 1, mRNA. |
| DDX60 | ILMN_1795181 | 0.00165 | 141803067 | 55601 | Homo sapiens DEAD (Asp-Glu-Ala-Asp) box |
| | | | | | polypeptide 60 (DDX60), mRNA. |
| ECGF1 | ILMN_1690939 | 0.00165 | 7669488 | 1890 | Homo sapiens endothelial cell growth factor 1 |
| | | | | | (platelet-derived) (ECGF1), mRNA. |
| LIMK2 | ILMN_2270443 | 0.00165 | 73390104 | 3985 | Homo sapiens LIM domain kinase 2 (LIMK2), |
| | | | | | transcript variant 2a, mRNA. |
| DOCK9 | ILMN_1773413 | 0.00165 | 24308028 | 23348 | Homo sapiens dedicator of cytokinesis 9 |
| | | | | | (DOCK9), mRNA. |
| EBI2 | ILMN_2168217 | 0.00165 | 50962860 | 1880 | Homo sapiens Epstein-Barr virus induced gene |
| | | | | | 2 (lymphocyte-specific G protein-coupled |
| | | | | | receptor) (EBI2), mRNA. |
| SUCNR1 | ILMN_1681601 | 0.00165 | 144922723 | 56670 | Homo sapiens succinate receptor 1 (SUCNR1), |
| | | | | | mRNA. |
| GZMK | ILMN_1710734 | 0.00164 | 73747815 | 3003 | Homo sapiens granzyme K (granzyme 3; |
| | | | | | tryptase II) (GZMK), mRNA. |
| KIAA1618 | ILMN_1674891 | 0.00162 | 113427610 | 57714 | PREDICTED: Homo sapiens KIAA1618 |
| | | | | | (KIAA1618), mRNA. |
| TNFAIP6 | ILMN_1785732 | 0.00157 | 26051242 | 7130 | Homo sapiens tumor necrosis factor, alpha- |
| | | | | | induced protein 6 (TNFAIP6), mRNA. |
| | ILMN_1903064 | 0.00156 | 27840194 | | BX116726 NCI_CGAP_Pr28 Homo sapiens |
| | | | | | cDNA clone IMAGp998J065569, mRNA |
| | | | | | sequence |
| SERPING1 | ILMN_1670305 | 0.00154 | 73858569 | 710 | Homo sapiens serpin peptidase inhibitor, clade |
| | | | | | G (C1 inhibitor), member 1, (angioedema, |
| | | | | | hereditary) (SERPING1), transcript variant 2, |
| | | | | | mRNA. |
| IFIH1 | ILMN_1781373 | 0.00154 | 27886567 | 64135 | Homo sapiens interferon induced with helicase |
| | | | | | C domain 1 (IFIH1), mRNA. |
| SIGLECP16 | ILMN_2229261 | 0.00151 | 84872113 | | Homo sapiens sialic acid binding Ig-like lectin, |
| | | | | | pseudogene 16 (SIGLECP16) on chromosome |
| | | | | | 19. |
| WDFY3 | ILMN_1697493 | 0.00146 | 31317267 | 23001 | Homo sapiens WD repeat and FYVE domain |
| | | | | | containing 3 (WDFY3), transcript variant 2, |
| | | | | | mRNA. |
| DYSF | ILMN_1810420 | 0.00146 | 19743938 | 8291 | Homo sapiens dysferlin, limb girdle muscular |
| | | | | | dystrophy 2B (autosomal recessive) (DYSF), |
| | | | | | mRNA. |
| CD28 | ILMN_1749362 | 0.00146 | 5453610 | 940 | Homo sapiens CD28 molecule (CD28), mRNA. |
| IFIT3 | ILMN_2239754 | 0.00139 | 31542979 | 3437 | Homo sapiens interferon-induced protein with |
| | | | | | tetratricopeptide repeats 3 (IFIT3), mRNA. |
| HIST2H2AA3 | ILMN_1659047 | 0.00139 | 21328454 | 8337 | Homo sapiens histone cluster 2, H2aa3 |
| | | | | | (HIST2H2AA3), mRNA. |
| ADM | ILMN_1708934 | 0.00138 | 4501944 | 133 | Homo sapiens adrenomedullin (ADM), mRNA. |
| ASPHD2 | ILMN_2167426 | 0.00138 | 29648312 | 57168 | Homo sapiens aspartate beta-hydroxylase |
| | | | | | domain containing 2 (ASPHD2), mRNA. |
| MGC52498 | ILMN_2185675 | 0.00138 | 111548661 | 348378 | Homo sapiens hypothetical protein MGC52498 |
| | | | | | (MGC52498), mRNA. |
| CTSL1 | ILMN_2374036 | 0.00138 | 125987604 | 1514 | Homo sapiens cathepsin L1 (CTSL1), transcript |
| | | | | | variant 2, mRNA. |
| GBP6 | ILMN_2121568 | 0.00137 | 38348239 | 163351 | Homo sapiens guanylate binding protein |
| | | | | | family, member 6 (GBP6), mRNA. |
| PIK3C2B | ILMN_2117323 | 0.00133 | 15451925 | 5287 | Homo sapiens phosphoinositide-3-kinase, class |
| | | | | | 2, beta polypeptide (PIK3C2B), mRNA. |
| SIRPG | ILMN_2383058 | 0.00126 | 94538336 | 55423 | Homo sapiens signal-regulatory protein gamma |
| | | | | | (SIRPG), transcript variant 2, mRNA. |
| ZDHHC19 | ILMN_1766896 | 0.00125 | 88900492 | 131540 | Homo sapiens zinc finger, DHHC-type |
| | | | | | containing 19 (ZDHHC19), mRNA. |
| IFI16 | ILMN_1710937 | 0.00125 | 5031778 | 3428 | Homo sapiens interferon, gamma-inducible |
| | | | | | protein 16 (IFI16), mRNA. |
| HPSE | ILMN_2092850 | 0.00124 | 94721346 | 10855 | Homo sapiens heparanase (HPSE), mRNA. |
| EPSTI1 | ILMN_2388547 | 0.00124 | 50428918 | 94240 | Homo sapiens epithelial stromal interaction 1 |
| | | | | | (breast) (EPSTI1), transcript variant 2, mRNA. |
| STOM | ILMN_1696419 | 0.00122 | 38016910 | 2040 | Homo sapiens stomatin (STOM), transcript |
| | | | | | variant 1, mRNA. |
| RAB20 | ILMN_1708881 | 0.0012 | 8923400 | 55647 | Homo sapiens RAB20, member RAS oncogene |
| | | | | | family (RAB20), mRNA. |
| IFI35 | ILMN_1745374 | 0.0012 | 34147320 | 3430 | Homo sapiens interferon-induced protein 35 |
| | | | | | (IFI35), mRNA. |
| SAMD9L | ILMN_1799467 | 0.0012 | 51339290 | 219285 | Homo sapiens sterile alpha motif domain |
| | | | | | containing 9-like (SAMD9L), mRNA. |
| PARP14 | ILMN_1691731 | 0.0012 | 50512291 | 54625 | Homo sapiens poly (ADP-ribose) polymerase |
| | | | | | family, member 14 (PARP14), mRNA. |
| LILRA5 | ILMN_2357419 | 0.0012 | 32895366 | 353514 | Homo sapiens leukocyte immunoglobulin-like |
| | | | | | receptor, subfamily A (with TM domain), |
| | | | | | member 5 (LILRA5), transcript variant 1, |
| | | | | | mRNA. |
| IFIT3 | ILMN_1664543 | 0.0012 | 72534657 | 3437 | Homo sapiens interferon-induced protein with |
| | | | | | tetratricopeptide repeats 3 (IFIT3), mRNA. |
| GCH1 | ILMN_2335813 | 0.00111 | 66932969 | 2643 | Homo sapiens GTP cyclohydrolase 1 (dopa- |
| | | | | | responsive dystonia) (GCH1), transcript variant |
| | | | | | 3, mRNA. |
| LMNB1 | ILMN_2126706 | 0.0011 | 27436949 | 4001 | Homo sapiens lamin B1 (LMNB1), mRNA. |
| | | | | | af01b06.s1 Human bone marrow stromal cells |
| | ILMN_1819953 | 0.00109 | 2433863 | | Homo sapiens cDNA clone IMAGE: 1027283 3, |
| | | | | | mRNA sequence |
| IFIT2 | ILMN_1739428 | 0.00107 | 153082754 | 3433 | Homo sapiens interferon-induced protein with |
| | | | | | tetratricopeptide repeats 2 (IFIT2), mRNA. |
| LAP3 | ILMN_1683792 | 0.00103 | 41393560 | 51056 | Homo sapiens leucine aminopeptidase 3 |
| | | | | | (LAP3), mRNA. |
| TLR5 | ILMN_1722981 | 0.000973 | 124248535 | 7100 | Homo sapiens toll-like receptor 5 (TLR5), |
| | | | | | mRNA. |
| TRAFD1 | ILMN_1758250 | 0.00097 | 5729827 | 10906 | Homo sapiens TRAF-type zinc finger domain |
| | | | | | containing 1 (TRAFD1), mRNA. |
| SCO2 | ILMN_1701621 | 0.00097 | 4826991 | 9997 | Homo sapiens SCO cytochrome oxidase |
| | | | | | deficient homolog 2 (yeast) (SCO2), nuclear |
| | | | | | gene encoding mitochondrial protein, mRNA. |
| TNFSF10 | ILMN_1801307 | 0.00097 | 23510439 | 8743 | Homo sapiens tumor necrosis factor (ligand) |
| | | | | | superfamily, member 10 (TNFSF10), mRNA. |
| DTX3L | ILMN_1784380 | 0.000959 | 31377615 | 151636 | Homo sapiens deltex 3-like (Drosophila) |
| | | | | | (DTX3L), mRNA. |
| CTSL1 | ILMN_1812995 | 0.000959 | 125987605 | 1514 | Homo sapiens cathepsin L1 (CTSL1), transcript |
| | | | | | variant 1, mRNA. |
| CREB5 | ILMN_1728677 | 0.000959 | 59938775 | 9586 | Homo sapiens cAMP responsive element |
| | | | | | binding protein 5 (CREB5), transcript variant 4, |
| | | | | | mRNA. |
| HIST2H2AC | ILMN_1768973 | 0.000955 | 27436923 | 8338 | Homo sapiens histone cluster 2, H2ac |
| | | | | | (HIST2H2AC), mRNA. |
| SESN1 | ILMN_1800626 | 0.000932 | 7657436 | 27244 | Homo sapiens sestrin 1 (SESN1), mRNA. |
| CEACAM1 | ILMN_2371724 | 0.000932 | 68161540 | 634 | Homo sapiens carcinoembryonic antigen- |
| | | | | | related cell adhesion molecule 1 (biliary |
| | | | | | glycoprotein) (CEACAM1), transcript variant |
| | | | | | 2, mRNA. |
| ZNF438 | ILMN_1678494 | 0.00091 | 33300650 | 220929 | Homo sapiens zinc finger protein 438 |
| | | | | | (ZNF438), mRNA. |
| C11orf75 | ILMN_1798270 | 0.000905 | 9910225 | 56935 | Homo sapiens chromosome 11 open reading |
| | | | | | frame 75 (C11orf75), mRNA. |
| HIST2H2AA3 | ILMN_2144426 | 0.000898 | 21328454 | 8337 | Homo sapiens histone cluster 2, H2aa3 |
| | | | | | (HIST2H2AA3), mRNA. |
| MAPK14 | ILMN_2388090 | 0.000869 | 20986513 | 1432 | Homo sapiens mitogen-activated protein kinase |
| | | | | | 14 (MAPK14), transcript variant 3, mRNA. |
| RTP4 | ILMN_2173975 | 0.000842 | 54607028 | 64108 | Homo sapiens receptor (chemosensory) |
| | | | | | transporter protein 4 (RTP4), mRNA. |
| LRFN3 | ILMN_2103919 | 0.000842 | 13375645 | 79414 | Homo sapiens leucine rich repeat and |
| | | | | | fibronectin type III domain containing 3 |
| | | | | | (LRFN3), mRNA. |
| PSME1 | ILMN_1726698 | 0.000842 | 30581140 | 5720 | Homo sapiens proteasome (prosome, |
| | | | | | macropain) activator subunit 1 (PA28 alpha) |
| | | | | | (PSME1), transcript variant 2, mRNA. |
| IL7R | ILMN_2342579 | 0.000842 | 28610150 | 3575 | Homo sapiens interleukin 7 receptor (IL7R), |
| | | | | | mRNA. |
| TAP2 | ILMN_1777565 | 0.000842 | 73747914 | 6891 | Homo sapiens transporter 2, ATP-binding |
| | | | | | cassette, sub-family B (MDR/TAP) (TAP2), |
| | | | | | transcript variant 1, mRNA. |
| FFAR2 | ILMN_1797895 | 0.000842 | 4885332 | 2867 | Homo sapiens free fatty acid receptor 2 |
| | | | | | (FFAR2), mRNA. |
| KREMEN1 | ILMN_1700994 | 0.000842 | 89191857 | 83999 | Homo sapiens kringle containing |
| | | | | | transmembrane protein 1 (KREMEN1), |
| | | | | | transcript variant 4, mRNA. |
| CENTA2 | ILMN_1763000 | 0.000842 | 93102369 | 55803 | Homo sapiens centaurin, alpha 2 (CENTA2), |
| | | | | | mRNA. |
| KCNJ15 | ILMN_1675756 | 0.000842 | 25777637 | 3772 | Homo sapiens potassium inwardly-rectifying |
| | | | | | channel, subfamily J, member 15 (KCNJ15), |
| | | | | | transcript variant 1, mRNA. |
| TRIM5 | ILMN_2404665 | 0.000842 | 15011945 | 85363 | Homo sapiens tripartite motif-containing 5 |
| | | | | | (TRIM5), transcript variant delta, mRNA. |
| UBE2L6 | ILMN_1769520 | 0.000842 | 38157980 | 9246 | Homo sapiens ubiquitin-conjugating enzyme |
| | | | | | E2L 6 (UBE2L6), transcript variant 1, mRNA. |
| FCER1G | ILMN_2123743 | 0.000817 | 4758343 | 2207 | Homo sapiens Fc fragment of IgE, high affinity |
| | | | | | I, receptor for; gamma polypeptide (FCER1G), |
| | | | | | mRNA. |
| PARP9 | ILMN_1731224 | 0.0008 | 13899296 | 83666 | Homo sapiens poly (ADP-ribose) polymerase |
| | | | | | family, member 9 (PARP9), mRNA. |
| PRRG4 | ILMN_1661809 | 0.0008 | 40255027 | 79056 | Homo sapiens proline rich Gla (G- |
| | | | | | carboxyglutamic acid) 4 (transmembrane) |
| | | | | | (PRRG4), mRNA. |
| CASP4 | ILMN_1778059 | 0.000767 | 73622124 | 837 | Homo sapiens caspase 4, apoptosis-related |
| | | | | | cysteine peptidase (CASP4), transcript variant |
| | | | | | gamma, mRNA. |
| MAFB | ILMN_1764709 | 0.000759 | 31652256 | 9935 | Homo sapiens v-maf musculoaponeurotic |
| | | | | | fibrosarcoma oncogene homolog B (avian) |
| | | | | | (MAFB), mRNA. |
| APOL1 | ILMN_1688631 | 0.000759 | 21735615 | 8542 | Homo sapiens apolipoprotein L, 1 (APOL1), |
| | | | | | transcript variant 2, mRNA. |
| | ILMN_1845037 | 0.000759 | 22658346 | | Homo sapiens cDNA clone IMAGE: 5277162 |
| GK | ILMN_1725471 | 0.000756 | 42794761 | 2710 | Homo sapiens glycerol kinase (GK), transcript |
| | | | | | variant 2, mRNA. |
| CHMP5 | ILMN_2094166 | 0.000751 | 20127557 | 51510 | Homo sapiens chromatin modifying protein 5 |
| | | | | | (CHMP5), mRNA. |
| ACTA2 | ILMN_1671703 | 0.000743 | 4501882 | 59 | Homo sapiens actin, alpha 2, smooth muscle, |
| | | | | | aorta (ACTA2), mRNA. |
| TIFA | ILMN_1686454 | 0.000709 | 38202233 | 92610 | Homo sapiens TRAF-interacting protein with |
| | | | | | forkhead-associated domain (TIFA), mRNA. |
| | ILMN_1859584 | 0.000699 | 10439674 | | Homo sapiens cDNA: FLJ23098 fis, clone |
| | | | | | LNG07440 |
| STAT1 | ILMN_1690105 | 0.000699 | 21536299 | 6772 | Homo sapiens signal transducer and activator of |
| | | | | | transcription 1, 91 kDa (STAT1), transcript |
| | | | | | variant alpha, mRNA. |
| SESTD1 | ILMN_1724495 | 0.000699 | 59709431 | 91404 | Homo sapiens SEC14 and spectrin domains 1 |
| | | | | | (SESTD1), mRNA. |
| STAT2 | ILMN_1690921 | 0.000699 | 38202247 | 6773 | Homo sapiens signal transducer and activator of |
| | | | | | transcription 2, 113 kDa (STAT2), mRNA. |
| CEACAM1 | ILMN_1716815 | 0.000699 | 68161540 | 634 | Homo sapiens carcinoembryonic antigen- |
| | | | | | related cell adhesion molecule 1 (biliary |
| | | | | | glycoprotein) (CEACAM1), transcript variant |
| | | | | | 2, mRNA. |
| SIGLEC5 | ILMN_1740298 | 0.000699 | 4502658 | 8778 | Homo sapiens sialic acid binding Ig-like lectin |
| | | | | | 5 (SIGLEC5), mRNA. |
| FCGR1A | ILMN_2176063 | 0.000643 | 24431940 | 2209 | Homo sapiens Fc fragment of IgG, high affinity |
| | | | | | Ia, receptor (CD64) (FCGR1A), mRNA. |
| LIMK2 | ILMN_2367671 | 0.000643 | 73390131 | 3985 | Homo sapiens LIM domain kinase 2 (LIMK2), |
| | | | | | transcript variant 2b, mRNA. |
| ATF3 | ILMN_2374865 | 0.000643 | 95102482 | 467 | Homo sapiens activating transcription factor 3 |
| | | | | | (ATF3), transcript variant 4, mRNA. |
| | ILMN_1851599 | 0.000643 | 27878199 | | BX110640 Soares_testis_NHT Homo sapiens |
| | | | | | cDNA clone IMAGp998B094156, mRNA |
| | | | | | sequence |
| Sep-04 | ILMN_1776157 | 0.000643 | 17986244 | 5414 | Homo sapiens septin 4 (SEPT4), transcript |
| | | | | | variant 2, mRNA. |
| STAT1 | ILMN_1777325 | 0.000643 | 21536299 | 6772 | Homo sapiens signal transducer and activator of |
| | | | | | transcription 1, 91 kDa (STAT1), transcript |
| | | | | | variant alpha, mRNA. |
| KIAA1618 | ILMN_2289093 | 0.000585 | 66529202 | 57714 | Homo sapiens KIAA1618 (KIAA1618), |
| | | | | | mRNA. |
| UBE2L6 | ILMN_1703108 | 0.000585 | 38157980 | 9246 | Homo sapiens ubiquitin-conjugating enzyme |
| | | | | | E2L 6 (UBE2L6), transcript variant 1, mRNA. |
| HPSE | ILMN_1779547 | 0.000574 | 19923365 | 10855 | Homo sapiens heparanase (HPSE), mRNA. |
| LACTB | ILMN_1693830 | 0.000562 | 26051232 | 114294 | Homo sapiens lactamase, beta (LACTB), |
| | | | | | nuclear gene encoding mitochondrial protein, |
| | | | | | transcript variant 2, mRNA. |
| FCGR1B | ILMN_2391051 | 0.000562 | 51972255 | 2210 | Homo sapiens Fc fragment of IgG, high affinity |
| | | | | | Ib, receptor (CD64) (FCGR1B), transcript |
| | | | | | variant 2, mRNA. |
| TRIM22 | ILMN_1779252 | 0.000562 | 117938315 | 10346 | Homo sapiens tripartite motif-containing 22 |
| | | | | | (TRIM22), mRNA. |
| DRAM | ILMN_1669376 | 0.000562 | 110825977 | 55332 | Homo sapiens damage-regulated autophagy |
| | | | | | modulator (DRAM), mRNA. |
| LOC728744 | ILMN_1654389 | 0.000562 | 113410932 | 728744 | PREDICTED: Homo sapiens hypothetical |
| | | | | | LOC728744 (LOC728744), mRNA. |
| PSTPIP2 | ILMN_1713058 | 0.000562 | 24850110 | 9050 | Homo sapiens proline-serine-threonine |
| | | | | | phosphatase interacting protein 2 (PSTPIP2), |
| | | | | | mRNA. |
| AIM2 | ILMN_1681301 | 0.000562 | 4757733 | 9447 | Homo sapiens absent in melanoma 2 (AIM2), |
| | | | | | mRNA. |
| SLC26A8 | ILMN_1755843 | 0.000562 | 20336283 | 116369 | Homo sapiens solute carrier family 26, member |
| | | | | | 8 (SLC26A8), transcript variant 1, mRNA. |
| FAM102A | ILMN_1745112 | 0.000562 | 78191786 | 399665 | Homo sapiens family with sequence similarity |
| | | | | | 102, member A (FAM102A), transcript variant |
| | | | | | 1, mRNA. |
| FBXO6 | ILMN_1701455 | 0.000554 | 48995170 | 26270 | Homo sapiens F-box protein 6 (FBXO6), |
| | | | | | mRNA. |
| LOC400759 | ILMN_1782487 | 0.000554 | 112734778 | | Homo sapiens similar to Interferon-induced |
| | | | | | guanylate-binding protein 1 (GTP-binding |
| | | | | | protein 1) (Guanine nucleotide-binding protein |
| | | | | | 1) (HuGBP-1) (LOC400759) on chromosome |
| | | | | | 1. |
| LHFPL2 | ILMN_1747744 | 0.000554 | 32698675 | 10184 | Homo sapiens lipoma HMGIC fusion partner- |
| | | | | | like 2 (LHFPL2), mRNA. |
| GBP1 | ILMN_1701114 | 0.000554 | 4503938 | 2633 | Homo sapiens guanylate binding protein 1, |
| | | | | | interferon-inducible, 67 kDa (GBP1), mRNA. |
| INCA | ILMN_1707979 | 0.000554 | 55925611 | 440068 | Homo sapiens inhibitory caspase recruitment |
| | | | | | domain (CARD) protein (INCA), mRNA. |
| GADD45B | ILMN_1718977 | 0.000554 | 86991435 | 4616 | Homo sapiens growth arrest and DNA-damage- |
| | | | | | inducible, beta (GADD45B), mRNA. |
| DHRS9 | ILMN_1733998 | 0.000554 | 40548399 | 10170 | Homo sapiens dehydrogenase/reductase (SDR |
| | | | | | family) member 9 (DHRS9), transcript variant |
| | | | | | 1, mRNA. |
| LOC440731 | ILMN_1683250 | 0.000554 | 113411754 | 440731 | PREDICTED: Homo sapiens hypothetical |
| | | | | | LOC440731, transcript variant 2 (LOC440731), |
| | | | | | mRNA. |
| SQRDL | ILMN_1667199 | 0.000554 | 52851410 | 58472 | Homo sapiens sulfide quinone reductase-like |
| | | | | | (yeast) (SQRDL), mRNA. |
| ACOT9 | ILMN_1658995 | 0.000554 | 81295403 | 23597 | Homo sapiens acyl-CoA thioesterase 9 |
| | | | | | (ACOT9), transcript variant 2, mRNA. |
| TAP1 | ILMN_1751079 | 0.000554 | 53759115 | 6890 | Homo sapiens transporter 1, ATP-binding |
| | | | | | cassette, sub-family B (MDR/TAP) (TAP1), |
| | | | | | mRNA. |
| ANKRD22 | ILMN_1799848 | 0.000554 | 154091031 | 118932 | Homo sapiens ankyrin repeat domain 22 |
| | | | | | (ANKRD22), mRNA. |
| C16orf7 | ILMN_1693630 | 0.000554 | 108860689 | 9605 | Homo sapiens chromosome 16 open reading |
| | | | | | frame 7 (C16orf7), mRNA. |
| PLAUR | ILMN_2408543 | 0.000554 | 53829377 | 5329 | Homo sapiens plasminogen activator, urokinase |
| | | | | | receptor (PLAUR), transcript variant 1, mRNA. |
| MAPK14 | ILMN_1737627 | 0.000554 | 4503068 | 1432 | Homo sapiens mitogen-activated protein kinase |
| | | | | | 14 (MAPK14), transcript variant 1, mRNA. |
| GK | ILMN_2393296 | 0.000554 | 42794762 | 2710 | Homo sapiens glycerol kinase (GK), transcript |
| | | | | | variant 1, mRNA. |
| GCH1 | ILMN_1812759 | 0.00052 | 66932971 | 2643 | Homo sapiens GTP cyclohydrolase 1 (dopa- |
| | | | | | responsive dystonia) (GCH1), transcript variant |
| | | | | | 4, mRNA. |
| DYNLT1 | ILMN_1678766 | 0.000499 | 5730084 | 6993 | Homo sapiens dynein, light chain, Tctex-type 1 |
| | | | | | (DYNLT1), mRNA. |
| FCGR1B | ILMN_2261600 | 0.000499 | 63055062 | 2210 | Homo sapiens Fc fragment of IgG, high affinity |
| | | | | | Ib, receptor (CD64) (FCGR1B), transcript |
| | | | | | variant 1, mRNA. |
| BATF2 | ILMN_1690241 | 0.000499 | 45238853 | 116071 | Homo sapiens basic leucine zipper transcription |
| | | | | | factor, ATF-like 2 (BATF2), mRNA. |
| ANKRD22 | ILMN_2132599 | 0.000499 | 21389370 | 118932 | Homo sapiens ankyrin repeat domain 22 |
| | | | | | (ANKRD22), mRNA. |
| GBP5 | ILMN_2114568 | 0.000499 | 31377630 | 115362 | Homo sapiens guanylate binding protein 5 |
| | | | | | (GBP5), mRNA. |
| GBP6 | ILMN_1756953 | 0.000499 | 38348239 | 163351 | Homo sapiens guanylate binding protein |
| | | | | | family, member 6 (GBP6), mRNA. |
| GBP1 | ILMN_2148785 | 0.000499 | 4503938 | 2633 | Homo sapiens guanylate binding protein 1, |
| | | | | | interferon-inducible, 67 kDa (GBP1), mRNA. |
| PHTF1 | ILMN_1803464 | 0.000499 | 5729975 | 10745 | Homo sapiens putative homeodomain |
| | | | | | transcription factor 1 (PHTF1), mRNA. |
| WDFY1 | ILMN_1676448 | 0.000499 | 51702527 | 57590 | Homo sapiens WD repeat and FYVE domain |
| | | | | | containing 1 (WDFY1), mRNA. |
| GBP2 | ILMN_1774077 | 0.000499 | 38327557 | 2634 | Homo sapiens guanylate binding protein 2, |
| | | | | | interferon-inducible (GBP2), mRNA. |
| SRBD1 | ILMN_1798827 | 0.000499 | 39841072 | 55133 | Homo sapiens S1 RNA binding domain 1 |
| | | | | | (SRBD1), mRNA. |
| TAP2 | ILMN_1759250 | 0.000499 | 73747916 | 6891 | Homo sapiens transporter 2, ATP-binding |
| | | | | | cassette, sub-family B (MDR/TAP) (TAP2), |
| | | | | | transcript variant 2, mRNA. |
| SORT1 | ILMN_1707077 | 0.000499 | 52352810 | 6272 | Homo sapiens sortilin 1 (SORT1), mRNA. |
| PSME2 | ILMN_1786612 | 0.000499 | 30410791 | 5721 | Homo sapiens proteasome (prosome, |
| | | | | | macropain) activator subunit 2 (PA28 beta) |
| | | | | | (PSME2), mRNA. |
| MAPK14 | ILMN_1788002 | 0.000499 | 20986511 | 1432 | Homo sapiens mitogen-activated protein kinase |
| | | | | | 14 (MAPK14), transcript variant 2, mRNA. |
| DHRS9 | ILMN_2384181 | 0.000499 | 40548399 | 10170 | Homo sapiens dehydrogenase/reductase (SDR |
| | | | | | family) member 9 (DHRS9), transcript variant |
| | | | | | 1, mRNA. |
| WARS | ILMN_2337655 | 0.000499 | 47419913 | 7453 | Homo sapiens tryptophanyl-tRNA synthetase |
| | | | | | (WARS), transcript variant 1, mRNA. |
| WARS | ILMN_1727271 | 0.000499 | 47419915 | 7453 | Homo sapiens tryptophanyl-tRNA synthetase |
| | | | | | (WARS), transcript variant 2, mRNA. |
| FLVCR2 | ILMN_2204876 | 0.000499 | 8923349 | 55640 | Homo sapiens feline leukemia virus subgroup C |
| | | | | | cellular receptor family, member 2 (FLVCR2), |
| | | | | | mRNA. |
| DUSP3 | ILMN_1797522 | 0.000499 | 37655179 | 1845 | Homo sapiens dual specificity phosphatase 3 |
| | | | | | (vaccinia virus phosphatase VH1-related) |
| | | | | | (DUSP3), mRNA. |
| FER1L3 | ILMN_1810289 | 0.000499 | 19718758 | 26509 | Homo sapiens fer-1-like 3, myoferlin (C. elegans) |
| | | | | | (FER1L3), transcript variant 2, |
| | | | | | mRNA. |
| APOL2 | ILMN_2325337 | 0.000499 | 22035652 | 23780 | Homo sapiens apolipoprotein L, 2 (APOL2), |
| | | | | | transcript variant beta, mRNA. |
| STAT1 | ILMN_1691364 | 0.000499 | 21536300 | 6772 | Homo sapiens signal transducer and activator of |
| | | | | | transcription 1, 91 kDa (STAT1), transcript |
| | | | | | variant beta, mRNA. |
| BRSK1 | ILMN_2185845 | 0.000499 | 24308325 | 84446 | Homo sapiens BR serine/threonine kinase 1 |
| | | | | | (BRSK1), mRNA. |
| JAK2 | ILMN_1683178 | 0.000499 | 13325062 | 3717 | Homo sapiens Janus kinase 2 (a protein |
| | | | | | tyrosine kinase) (JAK2), mRNA. |
| CEACAM1 | ILMN_1664330 | 0.000499 | 68161539 | 634 | Homo sapiens carcinoembryonic antigen- |
| | | | | | related cell adhesion molecule 1 (biliary |
| | | | | | glycoprotein) (CEACAM1), transcript variant |
| | | | | | 1, mRNA. |
| GBP4 | ILMN_1771385 | 0.000499 | 142368926 | 115361 | Homo sapiens guanylate binding protein 4 |
| | | | | | (GBP4), mRNA. |
| PSMB9 | ILMN_2376108 | 0.000499 | 73747923 | 5698 | Homo sapiens proteasome (prosome, |
| | | | | | macropain) subunit, beta type, 9 (large |
| | | | | | multifunctional peptidase 2) (PSMB9), |
| | | | | | transcript variant 1, mRNA. |
| IL15 | ILMN_1724181 | 0.000499 | 26787979 | 3600 | Homo sapiens interleukin 15 (IL15), transcript |
| | | | | | variant 3, mRNA. |
| MTHFD2 | ILMN_2405521 | 0.000499 | 94721351 | 10797 | Homo sapiens methylenetetrahydrofolate |
| | | | | | dehydrogenase (NADP+ dependent) 2, |
| | | | | | methenyltetrahydrofolate cyclohydrolase |
| | | | | | (MTHFD2), nuclear gene encoding |
| | | | | | mitochondrial protein, transcript variant 2, |
| | | | | | mRNA. |
| STX11 | ILMN_1720771 | 0.000499 | 33667037 | 8676 | Homo sapiens syntaxin 11 (STX11), mRNA. |
| GYG1 | ILMN_2230862 | 0.000499 | 20127456 | 2992 | Homo sapiens glycogenin 1 (GYG1), mRNA. |
| VAMP5 | ILMN_1809467 | 0.000499 | 31543930 | 10791 | Homo sapiens vesicle-associated membrane |
| | | | | | protein 5 (myobrevin) (VAMP5), mRNA. |
| APOL6 | ILMN_1687201 | 0.000499 | 87162462 | 80830 | Homo sapiens apolipoprotein L, 6 (APOL6), |
| | | | | | mRNA. |
| RHBDF2 | ILMN_1691717 | 0.000499 | 93352557 | 79651 | Homo sapiens rhomboid 5 homolog 2 |
| | | | | | (Drosophila) (RHBDF2), transcript variant 2, |
| | | | | | mRNA. |
| RHBDF2 | ILMN_2373062 | 0.000499 | 93352555 | 79651 | Homo sapiens rhomboid 5 homolog 2 |
| | | | | | (Drosophila) (RHBDF2), transcript variant 1, |
| | | | | | mRNA. |

A transcriptional signature in the blood of active TB patients from both intermediate burden (London) and high burden (South Africa) regions was indentified, which is distinct from the signatures of latent TB patients and healthy controls as shown by hierarchical clustering and blinded class prediction. The signature of latent TB displayed molecular heterogeneity. The number of latent patients showing a transcriptional signature similar to that of active TB, in two independent cohorts of patients, is consistent with the expected frequency of patients in that group who would progress to active disease¹⁰. Next, these profiles of latent TB represent for those patients who have either sub-clinical active disease or higher burden latent infection was determined, and therefore are at higher risk of progression to active disease^{11,24}.

The transcriptional signature of active TB correlates with the radiographic extent of disease.

It was clear from our results (FIG. 1) that there was molecular heterogeneity with respect to the transcriptional signature of active TB patients. Although the majority of patients demonstrated the same 393 gene expression profile, a few outliers were apparent, who either showed a distinct or weaker transcriptional profile. For example out of the 21 patients in the Test Set of the active TB group, 4 had profiles which did not cluster with the other active TB patients and were more in keeping with the profiles of healthy controls or latent TB patients (labelled , #, ▪, ♦ in FIG. 1b). These were the 4 active patients misclassified by the K-nearest neighbours algorithm as discussed above.

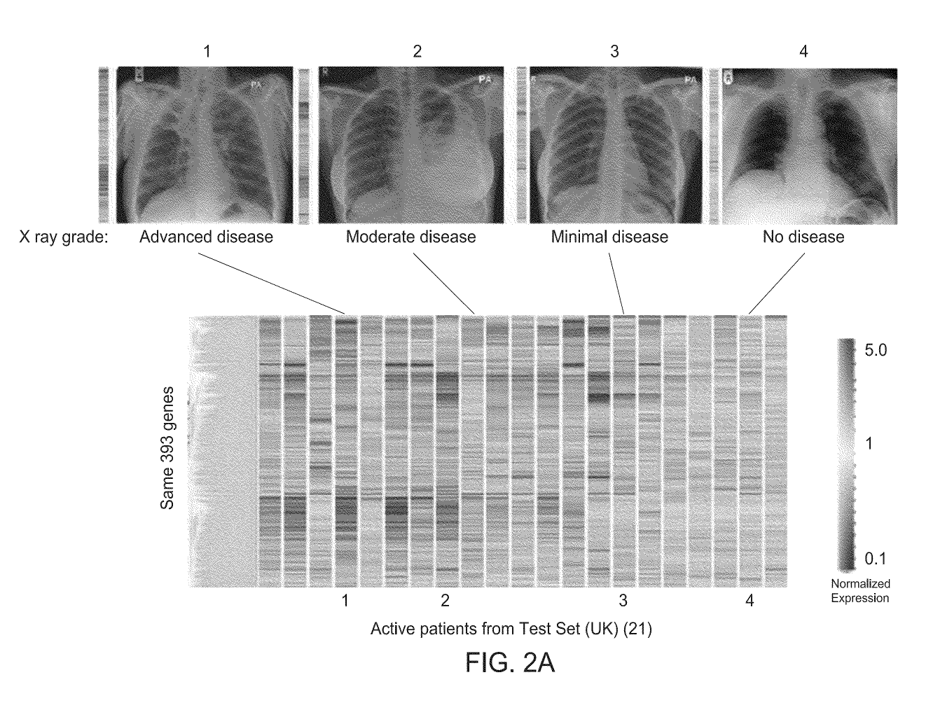

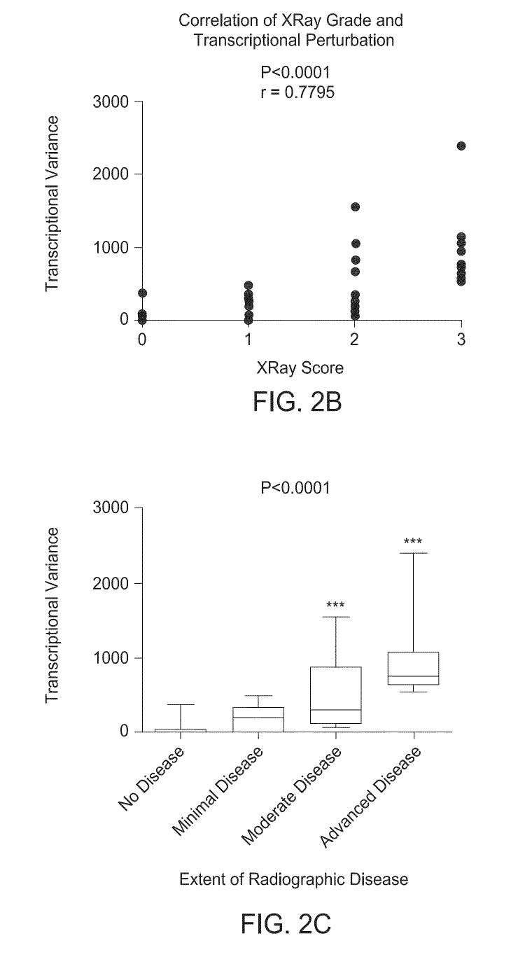

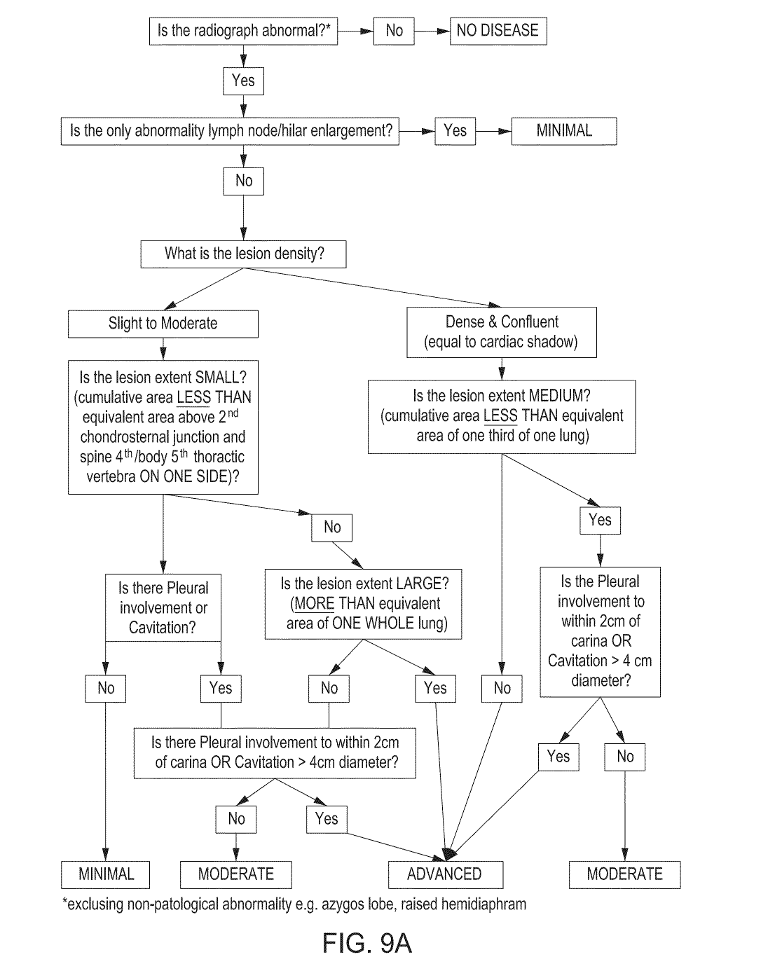

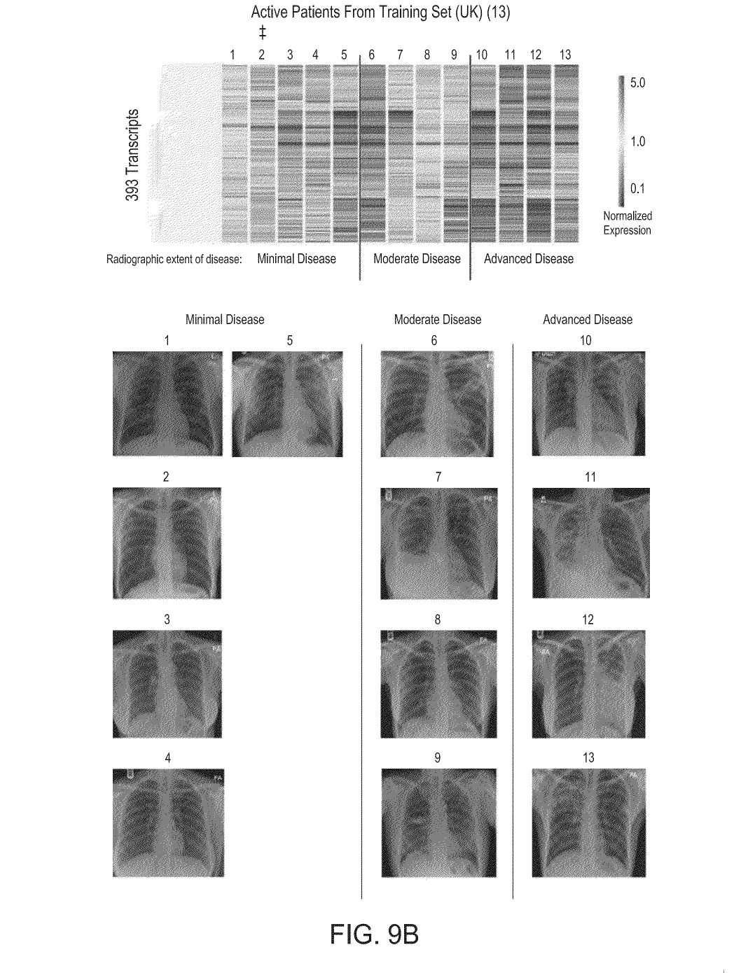

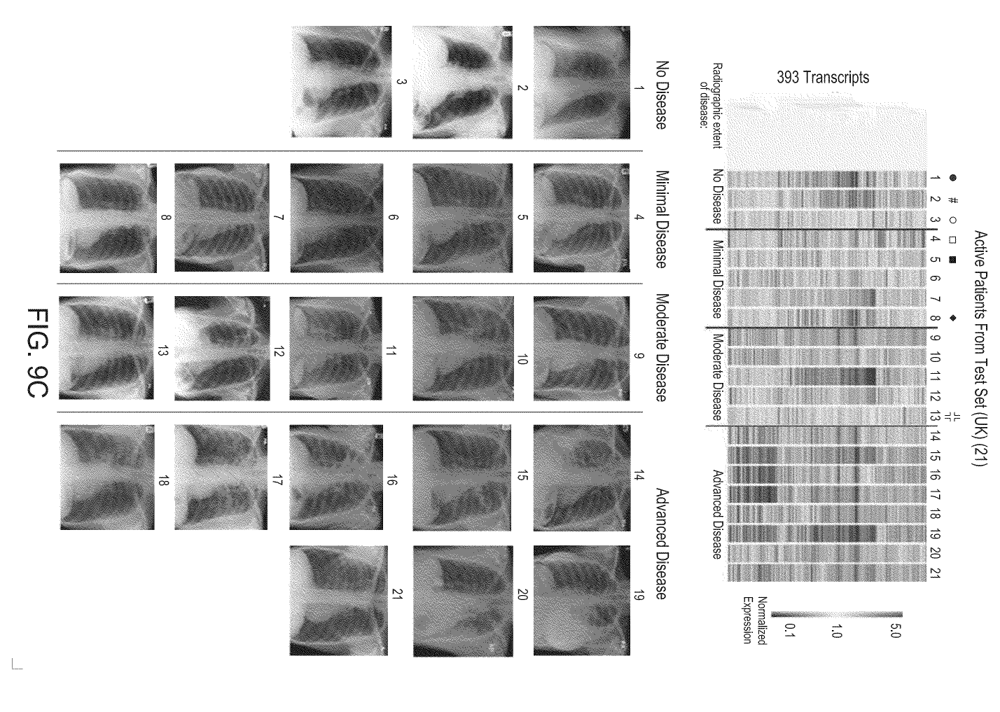

Molecular outliers in the active TB group could arise for a number of reasons. Firstly, there is the possibility of misdiagnosis, with false positive cultures arising from laboratory cross-contamination as previously reported²⁵. Alternatively the molecular/transcriptional heterogeneity could reflect heterogeneity in the extent of disease. To address this issue, chest radiographs taken at the time of diagnosis for each of the patients in the Training and Test Set were obtained, and graded by 2 chest physicians and a radiologist to assess the radiographic extent of disease. This assessment was performed without knowledge of the clinical diagnosis or transcriptional profile, using a modified version of the U.S. National Tuberculosis and Respiratory Disease Association Scheme, which classifies radiographic disease into no, minimal, moderately advanced, and far-advanced disease (Falk A, 1969; and FIG. 9a). The 393 transcript profiles for all 13 Active TB patients in the Training Set (FIG. 9b) and all 21 Active TB patients in the Test Set (FIG. 9c) were ordered in a heatmap according to their grade of radiographic extent of disease (Training Set, FIG. 9b; Test Set, FIG. 9c). This comparison of transcriptional profiles and radiographic grade, examples of which are shown in FIG. 2a, suggested that the transcriptional profile may correlate with extent of disease. To address this formally, we calculated a quantitative score of the molecular perturbation reflected by the transcriptional signature for each TB patient, the “Molecular Distance to Health”. This is a composite of both the number of transcripts in a profile that significantly differ from the healthy control baseline, and the degree of that difference²⁶. This score was calculated for each TB patients' 393-transcriptional profile and then compared with the radiographic grade for each latent (n=38) and active (n=30) TB patient in the Training and Test Sets. The scheme to assess radiographic extent of disease in this case is modified such that the radiographic extent of disease grade is converted to a numerical radiographic score. Profiles grouped according to radiographic extent of disease showed that mean “Molecular Distance to Health” increased with increasing radiographic extent of extent of disease (p<0.001 using Kruskal-Wallis ANOVA, with Dunn's multiple comparison post hoc testing to compare between groups) (FIG. 2b). These results show for the first time that the molecular signature in blood can provide a quantitative measure of extent of disease in active TB patients, and confirm that blood transcriptional profiles can reflect changes at the site of disease. Thus, using a systems biology approach, we identify a robust blood transcriptional signature for active pulmonary TB in both intermediate and high burden settings, which correlates with radiological extent of disease. This method can be used to monitor the extent of disease and possibly helpful in guiding treatment regimens.

Successful treatment diminishes the transcriptional signature of active TB.

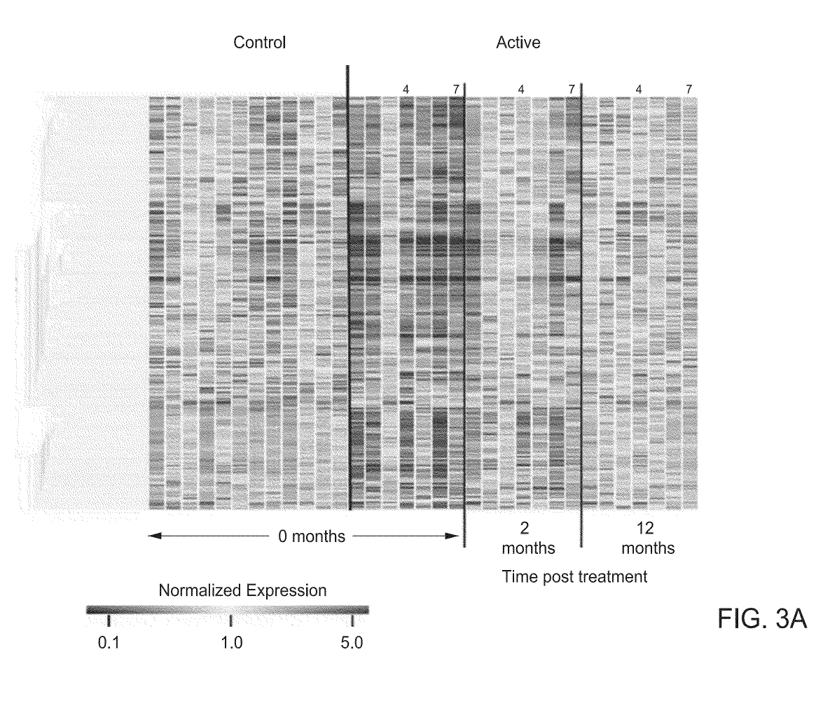

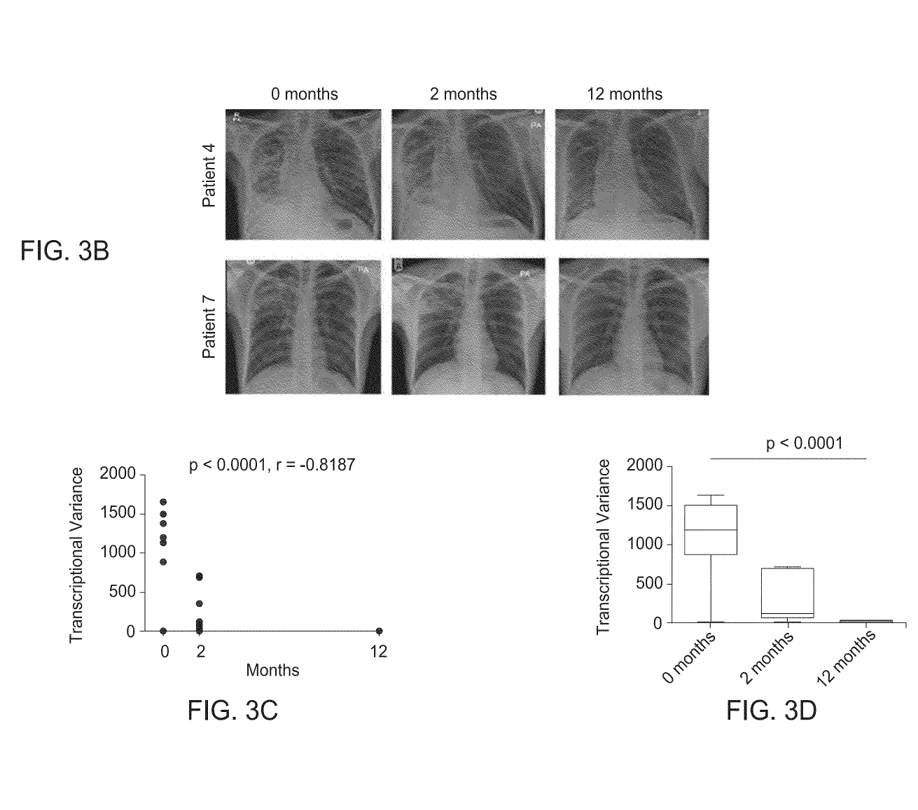

These findings demonstrate that the transcriptional signature of active TB correlates with the radiographic extent of disease it was of interest to determine whether the transcriptional signature would diminish during TB treatment and reflect efficacy of treatment. This would also confirm that this signature truly reflects TB disease. To test this, 7 patients with active TB were re-sampled at 2 and 12 months following initiation of anti-mycobacterial treatment, and their blood subjected again to microarray analysis as described earlier, together with their baseline pretreatment samples, and healthy control samples from the independent Test Set (n=12). The 393-transcript signature in active TB patients was again observed to be distinct from that of healthy controls (FIG. 3a). This transcriptional signature was diminished in most active TB patients after 2 months of treatment, and completely extinguished after 12 months of treatment, such that the active TB patients' signature started to resemble more closely that of healthy controls. This change in the transcriptional profile after 2 months of treatment was more pronounced in terms of the increased abundance of transcripts, which diminished in about 50% of the TB patients. This contrasted with the transcripts with decreased abundance, which were still present after 2 months of treatment, but returned to baseline expression after 12 months of treatment. The disappearance of the blood transcriptional signature during treatment of active TB patients appeared to reflect radiographic improvement (FIG. 3b). We next analysed the difference in the molecular distance to health score between each time point during treatment. The “Molecular Distance to Health” score of active TB patients at 12 months post treatment is significantly lower than at baseline pretreatment (p<0.001, Friedman Repeated Measures Test) (FIGS. 3c and d). These data suggest that the transcriptional signature in the blood of active TB patients may be used to monitor efficacy of treatment. Moreover it provides evidence that the 393-transcript signature is truly reflective of the host response to M. tuberculosis infection. Thus, the transcriptional signature of active TB is diminished during successful treatment, thereby providing a method to monitor quantitatively the response to anti-mycobacterial therapy, including clinical trials for new therapeutic agents.

TB patients in South Africa and London show the same modular signature.

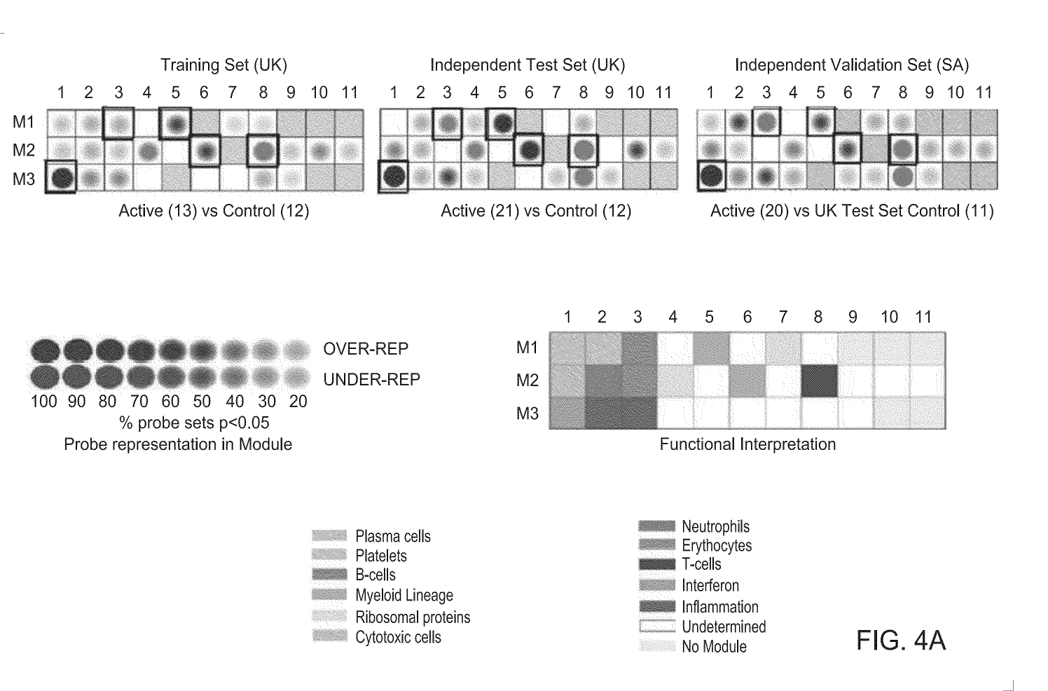

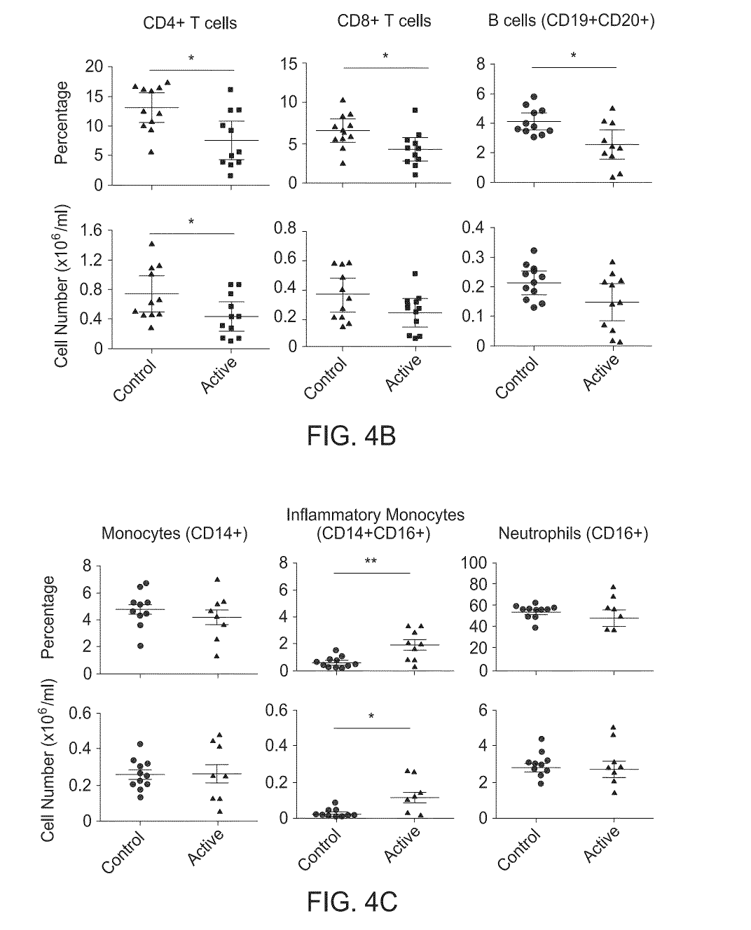

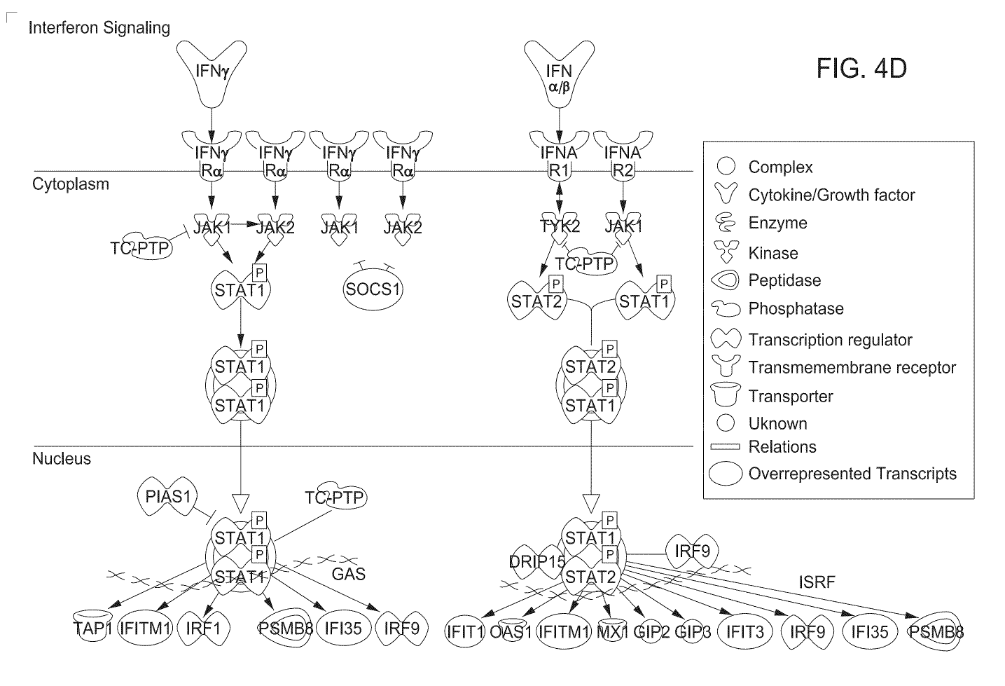

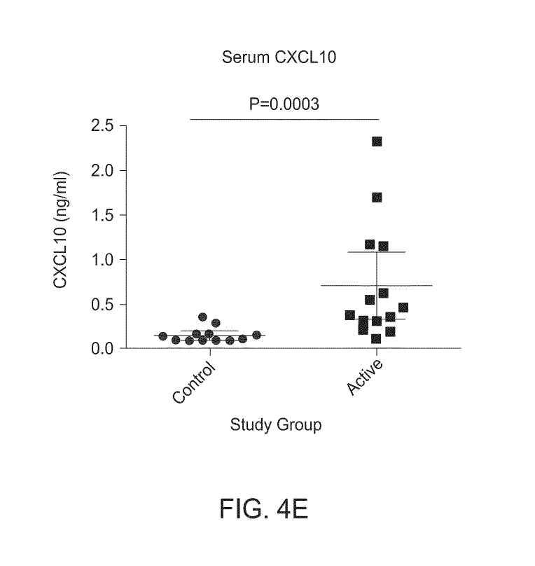

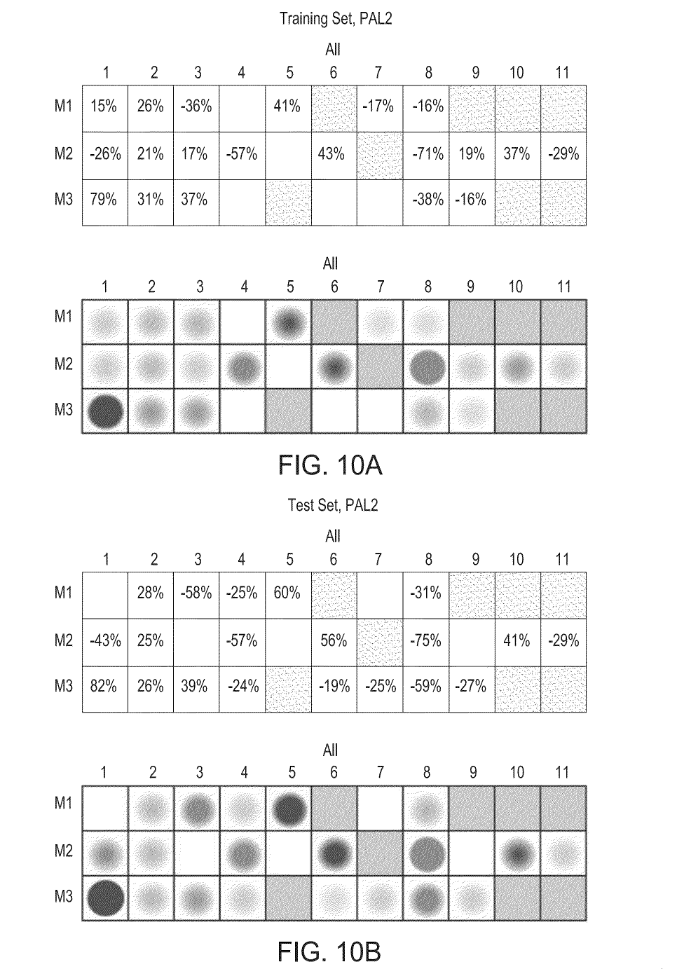

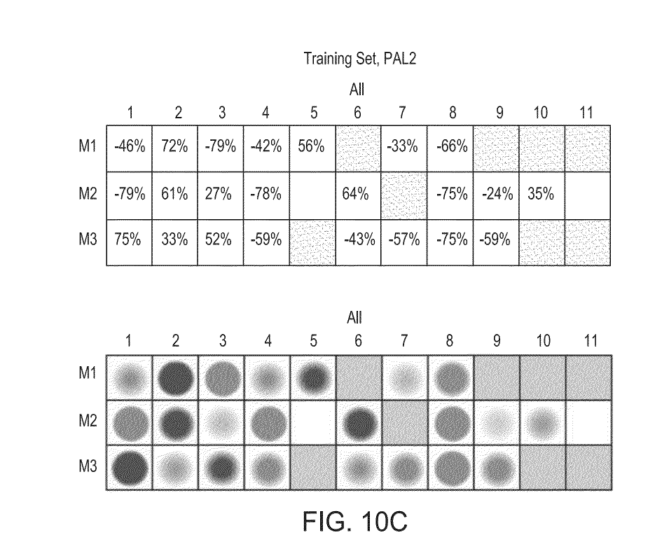

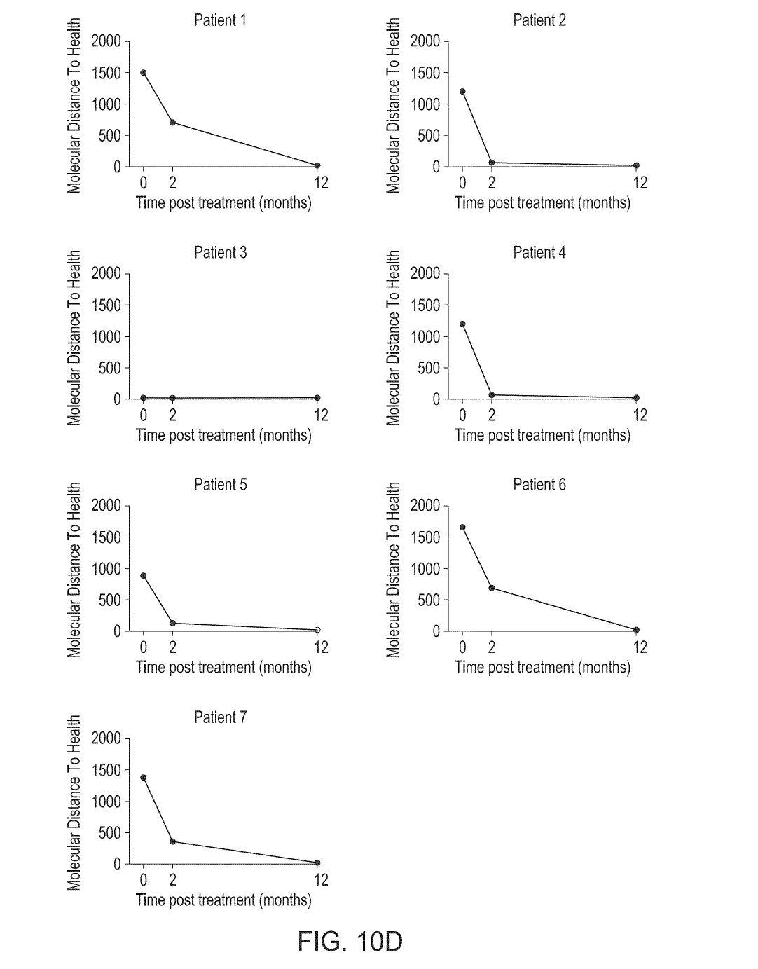

To expedite and focus the analysis of the transcriptional signature and characterize the host response during active TB disease, we employed a modular data mining strategy¹⁸. This strategy is based on observations that clusters of genes are coordinately expressed in a range of different inflammatory and infectious diseases. Discrete clusters of such genes can be defined as specific modules, which through unbiased literature profiling can often be shown to have a coherent functional relationship¹⁸. Modular analysis facilitated the evaluation and identification of changes in transcript abundance of functional relevance in the blood of active TB patients as compared to healthy controls (performed on the whole microarray dataset, filtering out only transcripts that were not detected (α=0.01) in at least 2 individuals) (FIG. 4a). The modular signature observed in the blood of active TB patients, (modules), was visually very similar for the London Training Set and Test Set and for the Independent South Africa Validation Set, as compared with healthy controls (FIG. 4a), confirming through an independent and unbiased analysis, the reproducibility of the transcriptional signature observed using classical clustering analysis (FIG. 1). The modular signature of active TB patients revealed decreased abundance of B cell (Module, M1.3) and T cell (Module, M2.8) related transcripts, and increased abundance of myeloid related transcripts (Modules, M1.5 and Modules, M2.6), and to a lesser extent increased abundance of neutrophil related transcripts (Module, M2.2). The largest proportion of transcripts changing in the blood of active TB patients as compared to controls were those within the interferon inducible (IFN) module (Module 3.1; 75-82% of the transcripts) (FIG. 4a; and FIGS. 10a-10c).

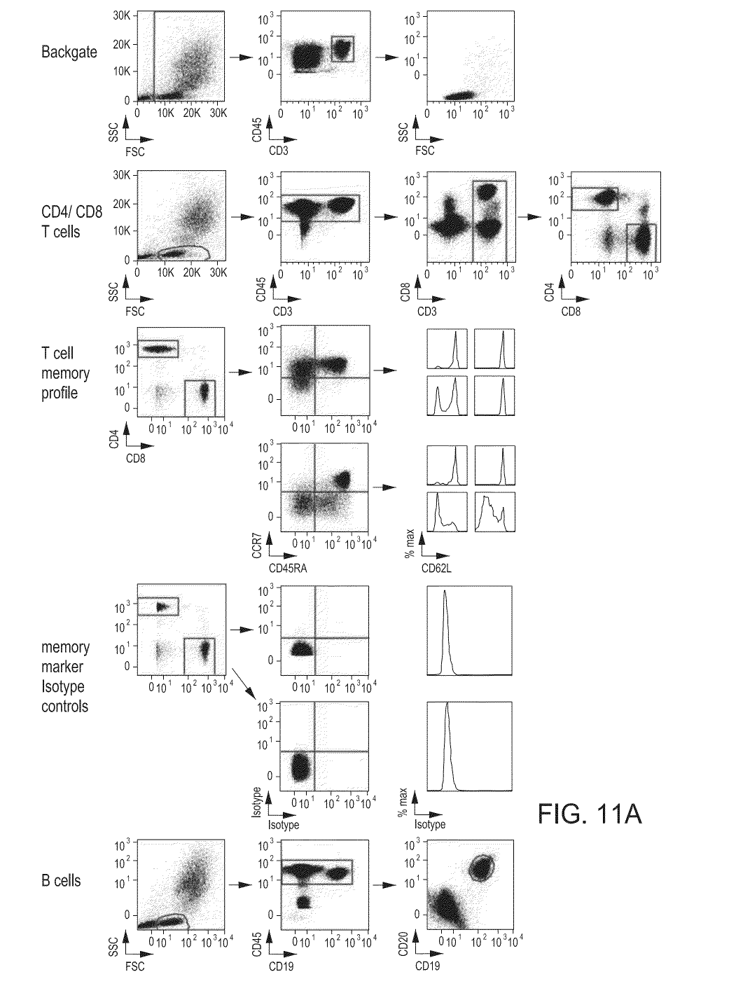

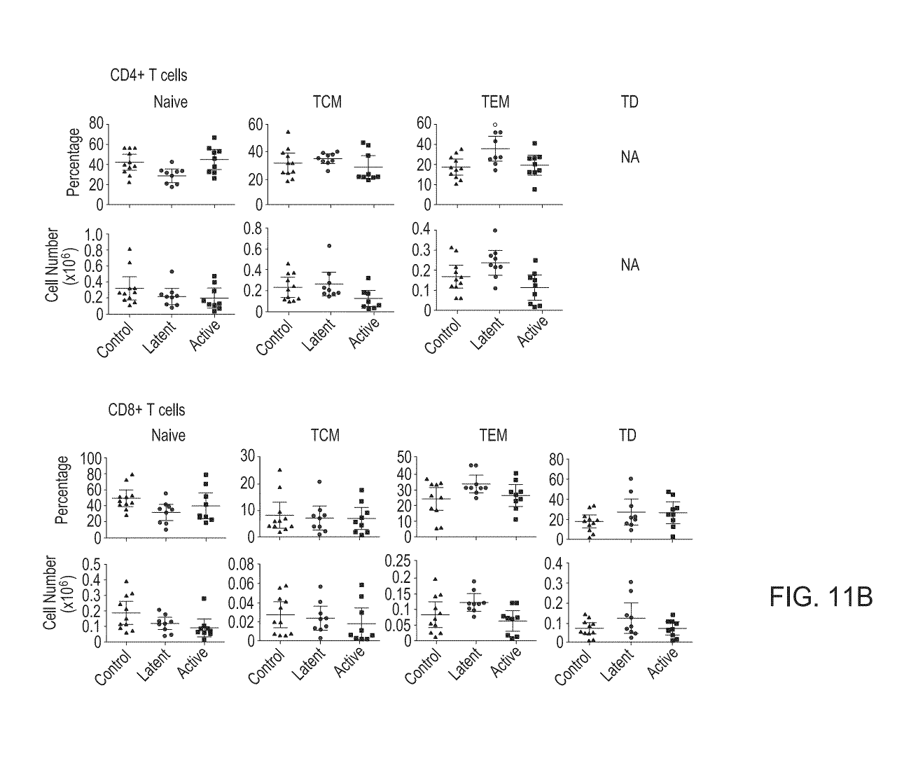

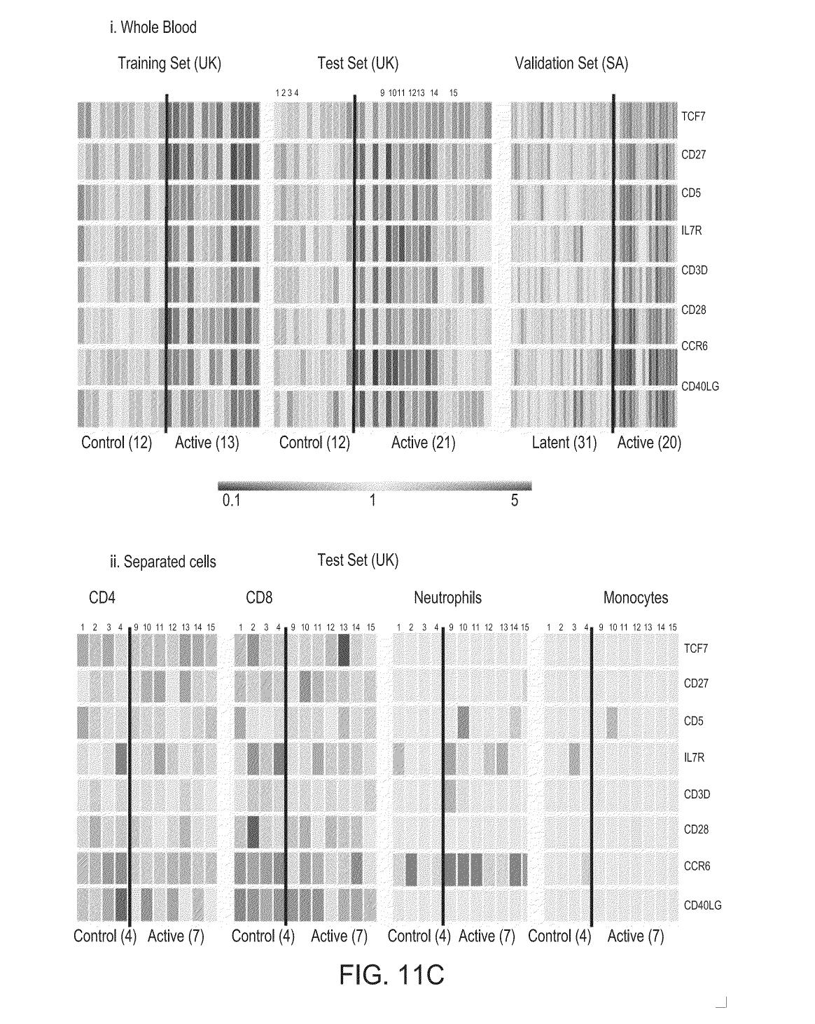

Blood is a heterogeneous tissue, therefore the transcriptional signature that we have defined in active TB patients could represent either changes in cell composition through migration, apoptosis or cellular proliferation, or changes in gene expression in discrete cellular populations. The total white blood cell/leucocyte counts in the blood of active TB patients were not significantly different from those in healthy controls (Student's t-test p=0.085). To address whether the apparent reduction in B and T cell transcripts revealed by the modular analysis (FIG. 4a) resulted from changes in cell numbers in the blood, and/or changes in gene expression in discrete cells, whole blood from the Test Set active TB patients and healthy controls was analysed by multi-parameter flow cytometry (FIG. 4b, FIGS. 11a and 11b). Both the percentages and numbers of CD4⁺ T cells and the percentages of CD8⁺ T cells and B cells were significantly reduced in the blood of active TB patients as compared to healthy controls (FIG. 4b). The reduction in the numbers of CD4⁺ T cells was largely attributable to significant decreases in numbers of central memory cells, with smaller but not significant effects on effector memory and naïve CD4⁺ T cells (FIG. 11b). However, decreases in CD8⁺ T cell numbers were mainly observed in the naïve T cell compartment. To confirm that the reduced transcriptional abundance of T cell related genes resulted from reduction in cell numbers rather than decreased expression of these genes, we assessed gene expression profiles for a number of representative T cell related genes in purified CD4⁺ and CD8⁺ T cells, as compared with whole blood (FIG. 11c). These T cell transcripts were shown to be less abundant in the whole blood of active TB patients as compared to healthy controls (FIG. 11c(i)). However, there was no difference in expression of these T cell-specific genes in CD4⁺ and CD8⁺ T cells purified from the blood of active TB patients as compared to those from healthy controls (FIG. 11c (ii)). Taken together, these data suggest that the lower transcriptional abundance of T cell genes in the blood of active TB patients results solely from reduction of cell numbers. In accordance with our findings, a number of studies have reported decreases in percentages and/or numbers of CD4⁺ T cells in the blood of active TB patients, although effects on CD8⁺ T cells and B cells were more varied^{27,28}. However the extent of this difference between TB patients and controls in our study suggests that this phenomenon extends beyond the migration of solely M. tuberculosis antigen-specific T cells, affecting a substantial proportion of the entire circulating T cell population.

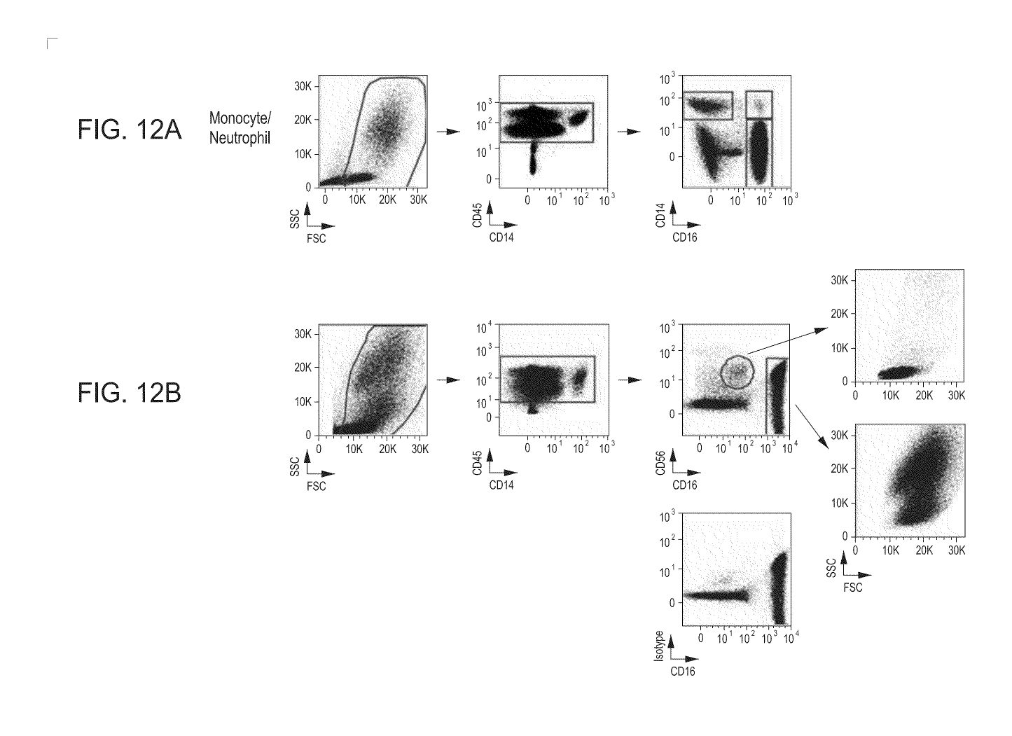

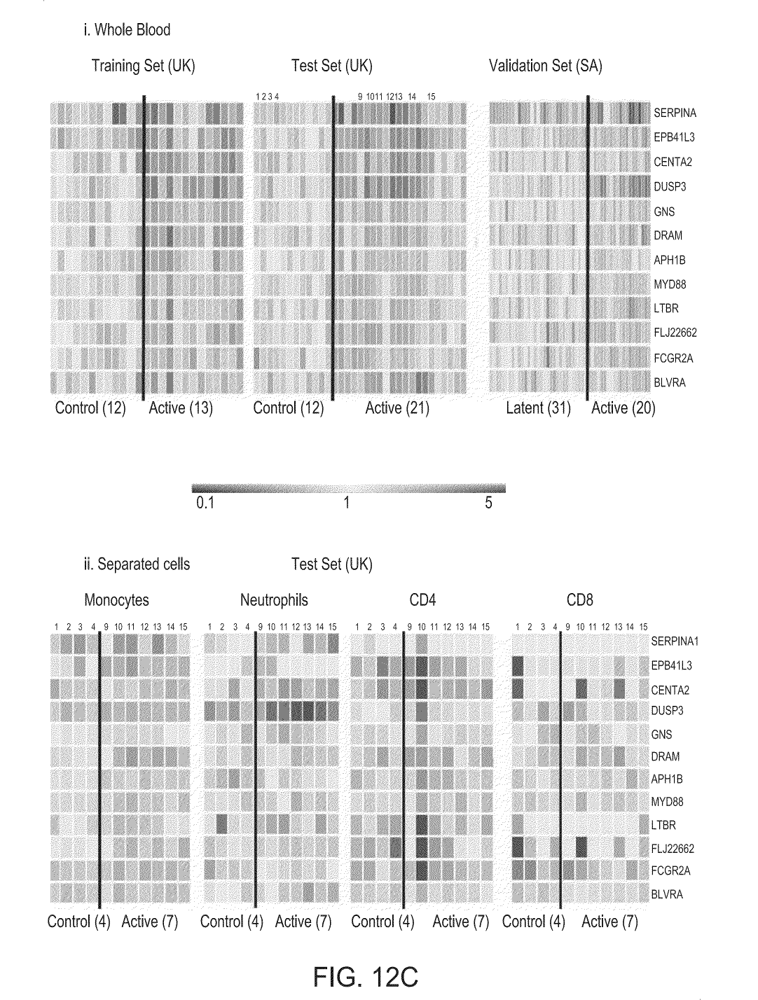

A substantial increase in myeloid cell-related transcripts at the modular level was observed in the active TB patients versus healthy controls for (Modules M1.5 and M2.6). To address whether this resulted from changes in cell number and/or changes in gene expression, whole blood was first analyzed for changes in myeloid type cells by flow cytometry (FIG. 12a). There was no change in monocyte (CD 14⁺, CD16⁻) or neutrophil (CD16⁺, CD14⁻) percentage or cell number in the blood of the Test Set Active TB patients compared with healthy controls (FIG. 4c). Of interest, a small but significant increase in the percentage and cell number of inflammatory monocytes (CD14⁺, CD16⁺), was observed in the blood of active TB patients as compared to healthy controls. Representative myeloid cell related transcripts were shown to be over-abundant in the blood of active TB patients versus healthy controls (FIG. 12b(i)). This increase was much less pronounced in purified monocytes (CD14⁺) (FIG. 12b(ii)), although the increased expression of these myeloid-related transcripts could have been diluted out if their increased expression was restricted to a small monocytic population, such as the CD14^{k}, CD16⁺ inflammatory subset. Inflammatory monocytes have previously been suggested to be increased in inflammatory and infectious diseases²⁹. Thus, the changes in the myeloid module can to some extent be explained by changes in gene expression, but may result from changes in numbers of inflammatory monocytes in the blood of active TB patients versus controls.

Interferon-inducible gene expression in neutrophils dominates the TB signature.

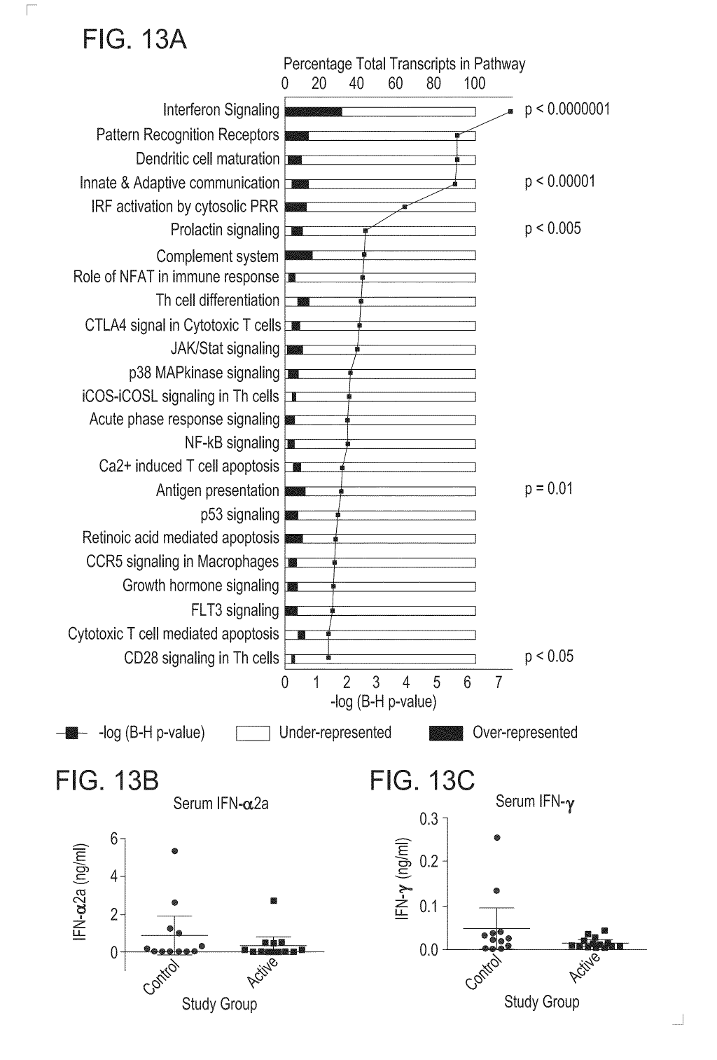

To confirm the over-representation of the IFN-inducible genes in the active TB patients shown by the modular analysis (FIG. 4a) transcripts constituting the 393 transcript signature were analysed using Ingenuity Pathways Analysis software. IFN signalling was confirmed as the most significantly over-represented functional pathway in the 393 transcripts using Fischer's Exact test with a Benjamini-Hochberg multiple test correction (p<0.0000001) as compared to other curated biological pathways generated from the literature (FIG. 13). Interestingly, genes downstream of both IFN-γ and Type I IFN α/β receptor signalling were significantly over-represented (marked in red in FIG. 4d) in the blood of active TB patients. It is of note that although neither IFN-α2a nor IFN-γ proteins were detectable in the serum of active TB patients (FIGS. 13b and 13c), elevated levels of the IFN-inducible chemokine CXCL10 (IP10) were detected in the blood of active TB patients versus controls (FIG. 4e).

Although IFN-γ has been shown to be protective during immune responses to intracellular pathogens, including mycobacteria^{14-16,30}, the role of Type I IFN is less clear. Signalling through the Type I IFNR (IFN-αβR) is crucial for defense against viral infections³¹, however IFN-αβ have been shown to be detrimental during intracellular bacterial infections³²⁻³⁴. However, the role of IFN-αβ in TB infection is unclear; many papers suggest a harmful role³⁵⁻³⁷; though others do not^{38,39}. There are a few case reports suggesting an association between IFN-α treatment for hepatitis C viral infection and M. tuberculosis infection^{40,41}.

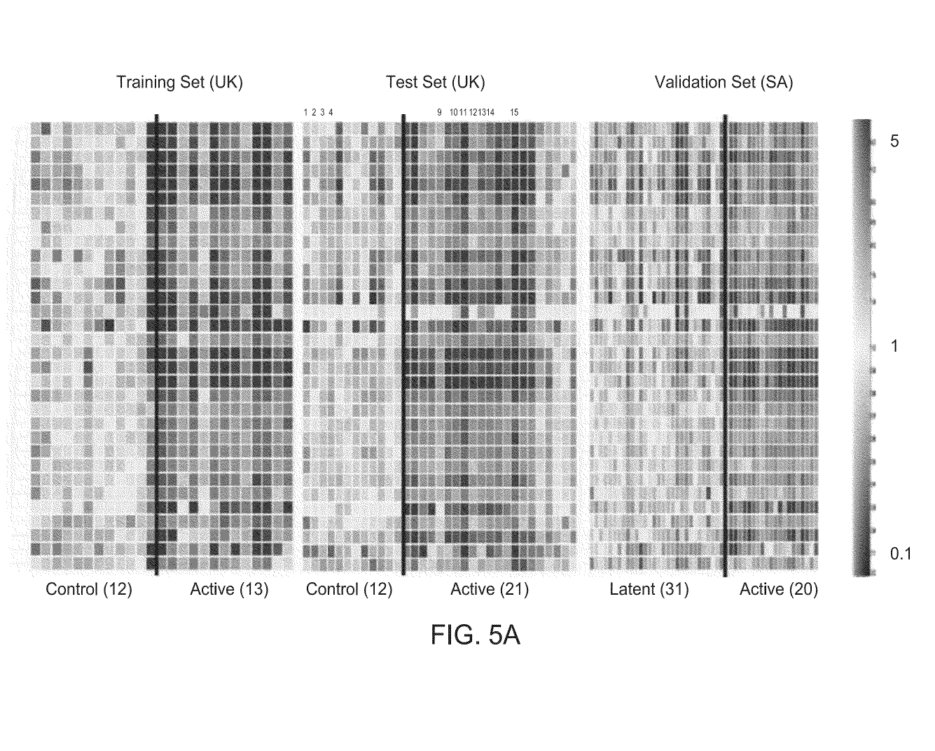

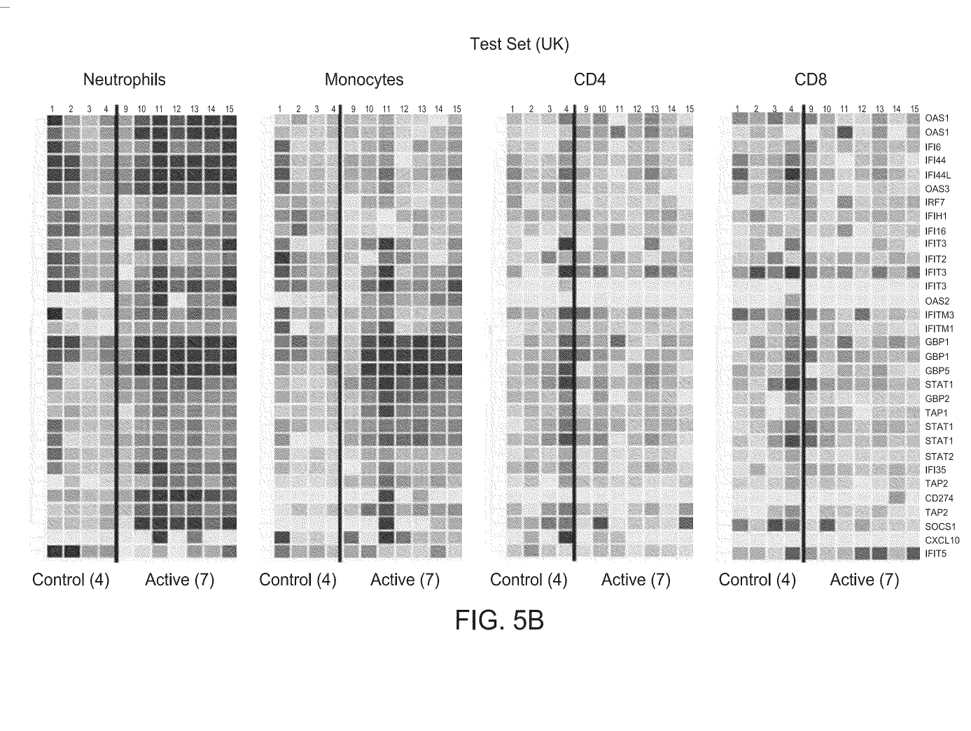

To determine whether the high transcriptional abundance of IFN-inducible genes in the blood of active TB patients was attributable to a particular cell type, we assessed the expression of genes for both the IFN-γ and Type I IFN α/β receptor signalling pathways, in purified neutrophils, monocytes and CD4⁺ and CD8⁺ T cells, as compared with whole blood (FIG. 5). A representative set of IFN-inducible transcripts was shown to be more abundant in the whole blood of active TB patients as compared to healthy controls (FIG. 5a). Strikingly, the IFN-inducible transcripts were shown to be substantially over-expressed in neutrophils and to a lesser extent monocytes purified from the blood of active TB patients as compared to the equivalent cells from healthy controls (FIG. 5b). In contrast, CD4⁺ and CD8⁺ T cells purified from blood of active TB patients showed no difference in expression of these IFN-inducible genes as compared to those purified from healthy control individuals (FIG. 5b).

Neutrophils are professional phagocytes which have been demonstrated to be the predominant cell type infected with rapidly replicating M. tuberculosis in TB patients⁴². The prevalence and responses of neutrophils in genetically susceptible mice as compared to resistant mice has led to the theory that neutrophils in TB inflammation contribute to pathology, rather than protection of the host⁴³. Our studies support a role for neutrophils in the pathogenesis of TB. This may result from their over-activation by both IFN-γ and Type I IFNs, which we now show to be a dominant transcriptional signature in blood of active TB patients, mainly expressed in neutrophils (FIG. 5).

PDL-1 is over-expressed by neutrophils in patients with active TB.

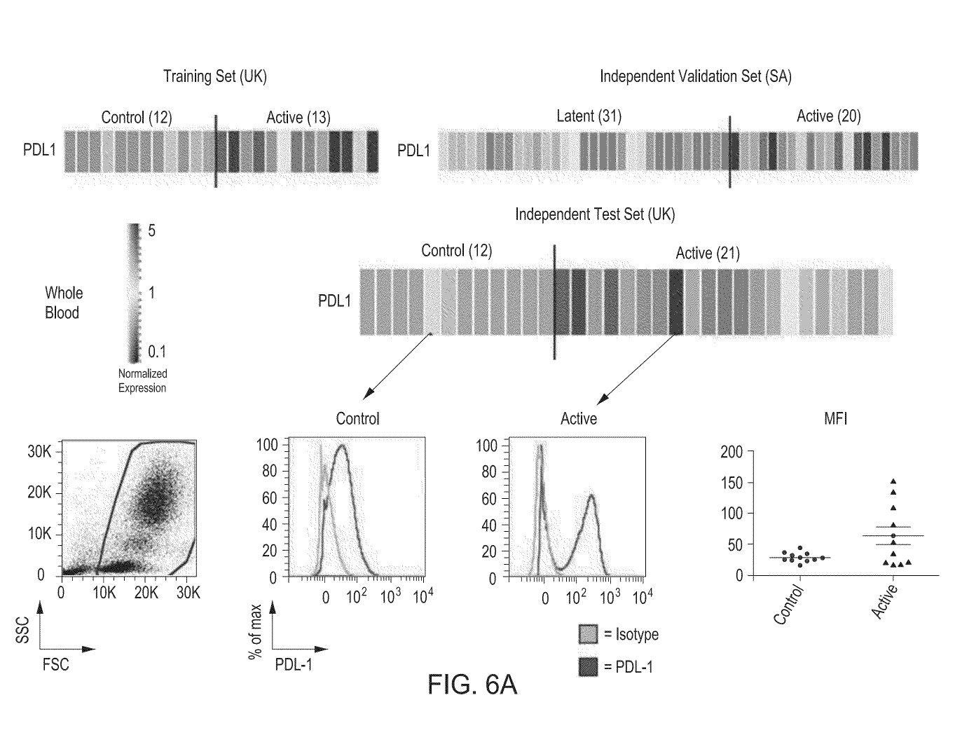

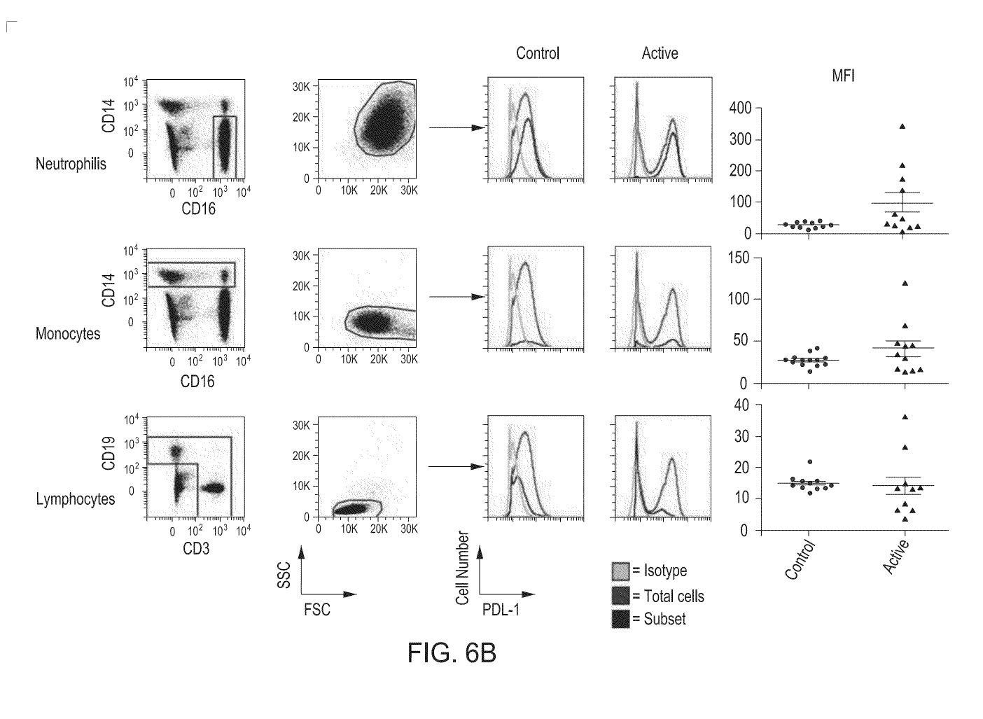

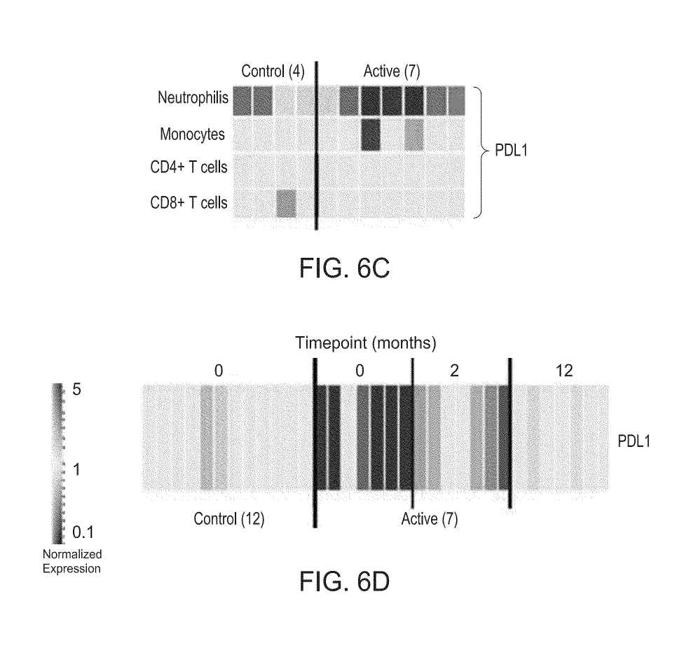

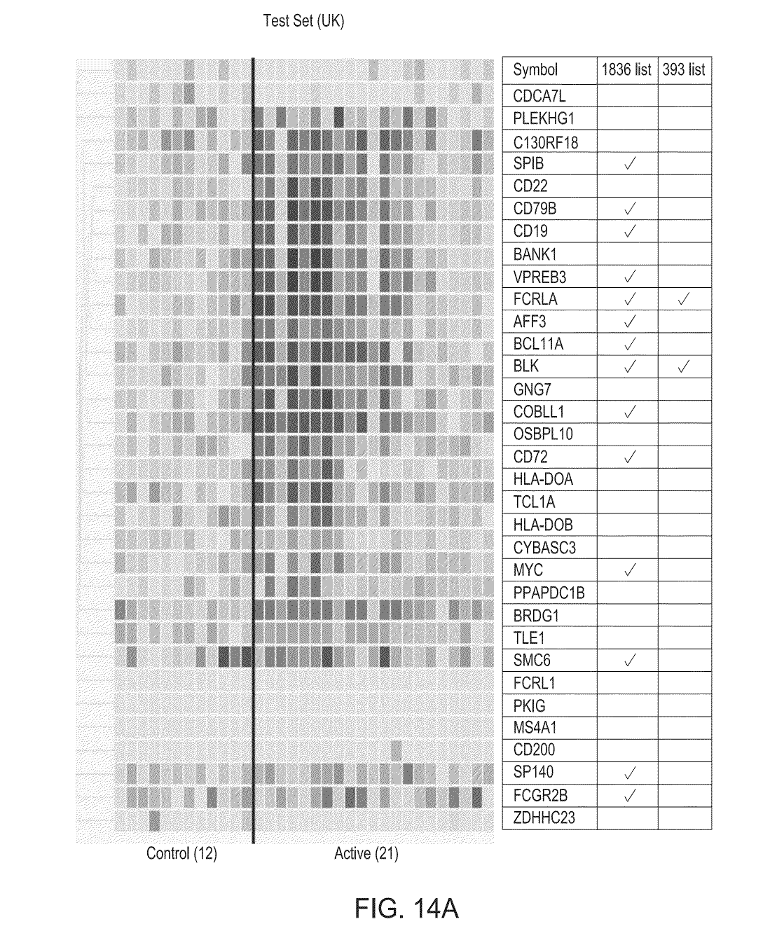

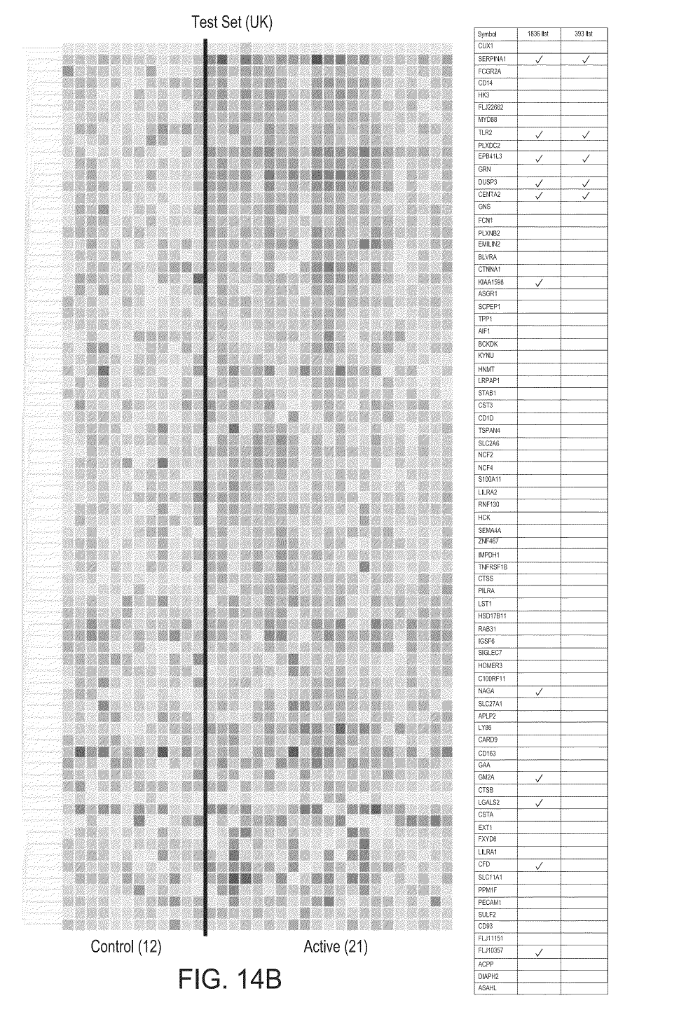

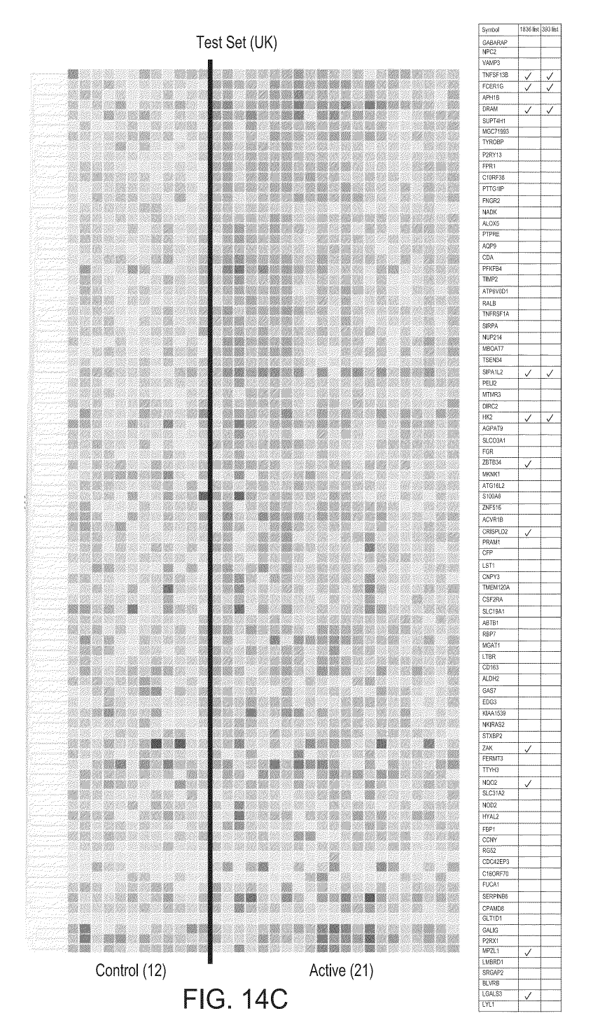

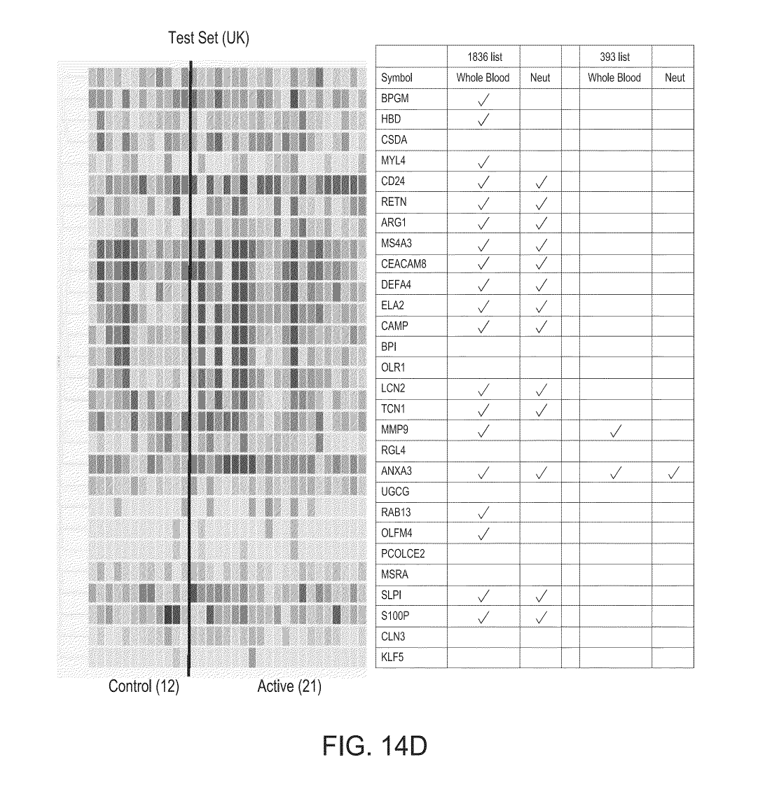

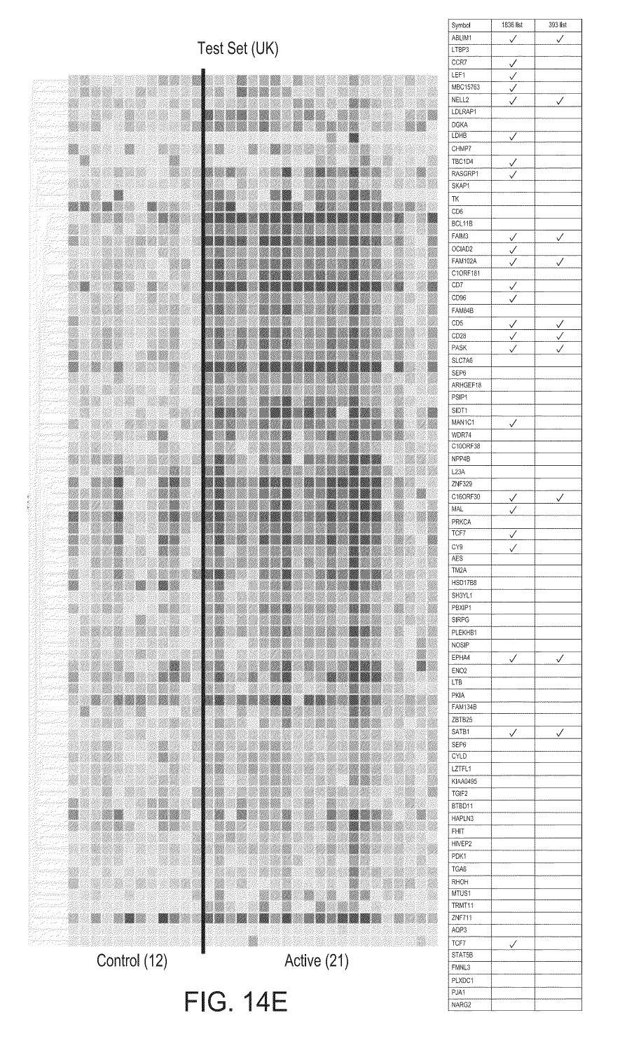

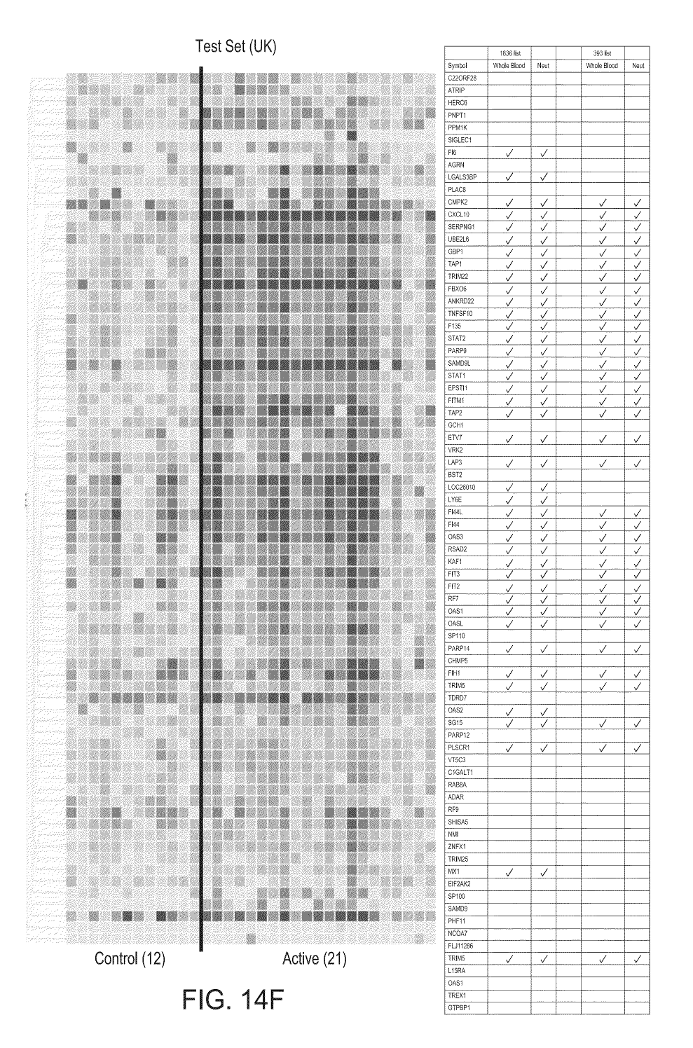

One gene with increased abundance in the blood of active TB patients clustering with the IFN-inducible transcripts was Programmed Death Ligand 1 (PDL-1, also denoted as CD274 and B7-H1), an immunoregulatory ligand expressed on diverse cells (FIG. 6). PDL-1 has been reported to suppress T cell proliferation and effector function, through binding the programmed death-1 receptor (PD-1), in chronic viral infections^{44,45}. To determine what cell may be over-expressing PDL-1, whole blood populations from active TB patients and healthy controls were analysed by flow cytometry, and PDL-1 was shown to be upregulated on whole leucocytes of patients with active TB as compared to controls/latent in Validation (SA) Set (FIG. 6a and FIG. 14). Increased PDL-1 expression was most evident on neutrophils, to a lesser extent on monocytes and was not evident on lymphocytes from active TB patients (FIG. 6b and FIG. 14). In keeping with these findings by flow cytometry, purified neutrophils from active TB patients expressed higher levels of PDL-1 transcripts, than in neutrophils from healthy controls. In contrast PDL-1 was only expressed in monocytes from 2 out of 7 active TB patients, and there was no detectable expression in T cells (FIG. 6c). The increased abundance of PDL-1 transcripts in the blood of active TB patients disappeared after successful therapy, although was still present at 2 months into treatment in the majority of patients (FIG. 6d).

These findings demonstrate that the presence of PDL-1 in the blood of active TB patients may be related to pathology and failure to control disease, consistent with reports in chronic viral infection^{44,45}. Furthermore, PD-1 expression has been reported to be increased on human T cells from TB patients, stimulated with sonicated H37Rv M. tuberculosis, and blocking antibodies to PDL-1/PD-1 were able to enhance antigen-specific IFN-γ and cytotoxic CD8⁺ T responses⁴⁶. Of relevance to our findings, HIV induced PDL-1 expression on monocytes and CCR5⁺ T cells have been shown to be dependent on IFN-α but not IFN-γ⁴⁷. Thus increased expression of PDL-1 in response to type I interferons in neutrophils, as we show here, could be one way in which over-expression of interferons could be detrimental to host responses. Whether blockade of PDL-1/PD-1 signalling may lead to enhanced protective responses may depend on the type and stage of infection/vaccination^{48,49}, and may require targeting the blockade to particular cells and sites, to achieve enhanced protection whilst avoiding immunopathology⁴⁴. The effect of PDL-1 on the immune response during bacterial infection may therefore be more complicated than at first thought, which is supported by our findings that PDL-1 is highly expressed on neutrophils but not T cells or monocytes in the blood of active TB patients.

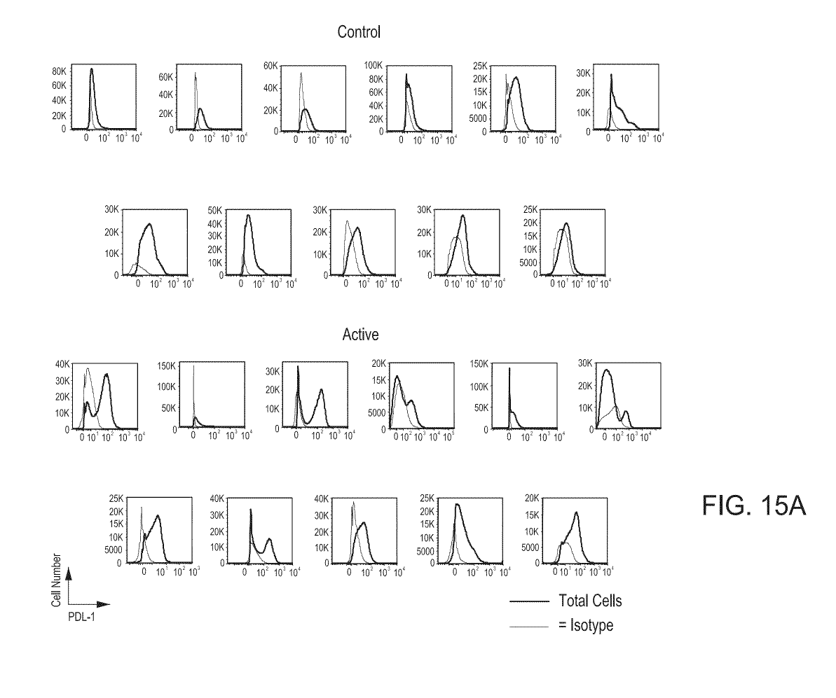

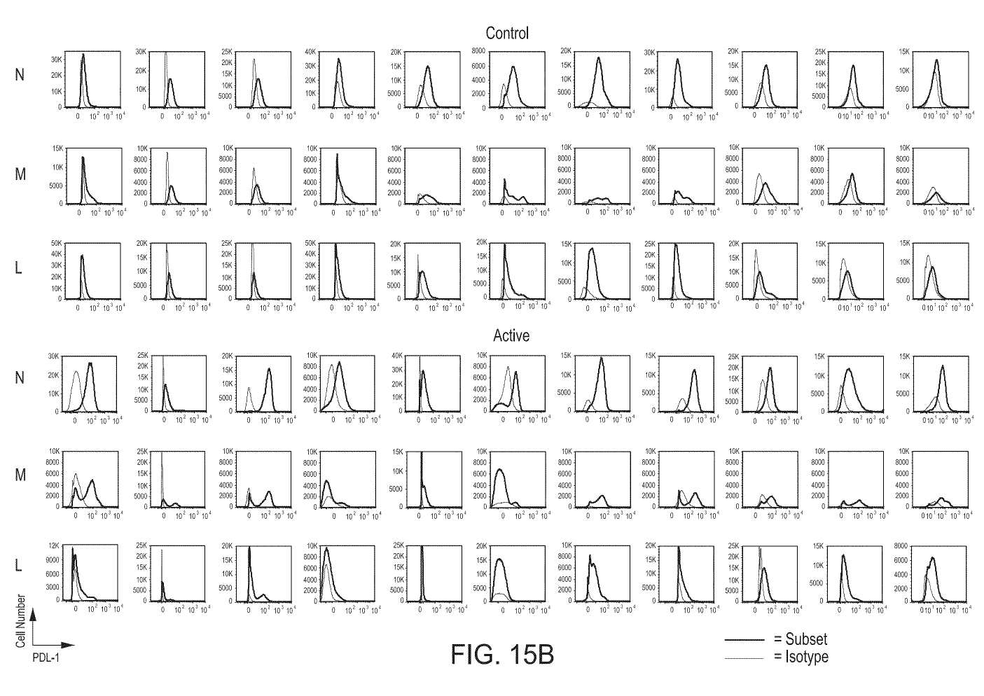

Improved understanding of the host response in TB is essential for improved diagnosis, vaccination and therapy (Young et al., 2008, JCI). Insight into this complex disease has been impaired for a number of reasons, including the fact that clinically defined latent TB actually represents a spectrum that runs from elimination of live mycobacteria to subclinical disease (Young et al., 2009, Trends Micro). Here we have defined a 393-gene transcriptional signature (FIG. 1 and FIG. 15) of active TB in the blood of patients from London and South Africa that is absent in the majority of latent TB patients and healthy controls. Furthermore, using this approach, and analysis of the required number of TB patients and healthy controls to achieve significance, we were able to demonstrate heterogeneity of the disease. For example, the signature of active TB was also observed in the blood of 10% of latent TB patients possibly revealing those individuals who may in the future develop active disease. This is the first molecular evidence that demonstrates the heterogeneity of TB, suggesting that this molecular approach may be useful in determining which individuals with latent TB should be given anti-mycobacterial chemotherapy. Future longitudinal studies are required to confirm that this signature is indeed predictive of future TB disease in latent patients.

The size and complexity of microarray data generated makes interpretation difficult, often forcing scientists to focus on a handful of candidate genes for further study^{50,51}, which may not be sufficient as specific biomarkers for diagnosis, and provide little information with respect to disease pathogenesis. To improve our understanding of the host factors underlying pathogenesis of TB we employed three distinct yet complementary analytical approaches, modular, pathway and gene level analysis, in order to yield insight into the biological pathways revealed by the transcriptional signature. Each approach identified common biological pathways involved in the host transcriptional response to M. tuberculosis and identified IFN-inducible genes as forming a key part of the immune signature in active pulmonary TB. We employed modular analysis first, as this is the most unsupervised approach and therefore least prone to bias. Modules were derived from multiple independent datasets and annotated by literature profiling, powerfully integrating both experimental data and knowledge from the accumulated literature¹⁸. This modular analysis revealed a dominant IFN-inducible signature of active TB disease. This was validated by an independent approach using Ingenuity Pathways analysis, which is entirely derived from published literature and confirmed the dominance of the IFN-inducible signature and further revealed that it consisted of IFN-γ and Type I IFN-inducible genes. Since the two approaches analyze different lists of transcripts, the identification of common biological processes by both methods confirms the robustness of our findings. As a further level of validation, individual gene level analysis corroborated but also expanded upon the findings from the other analytical methods. Using these approaches and further immunological analyses we revealed the key components of the host blood transcriptional response to M. tuberculosis as a neutrophil-driven IFN-inducible signature, which is extinguished by successful treatment. This study improves our understanding of the fundamental biology of TB and may offer future leads for diagnosis and treatment.

Blood represents a reservoir and a migration compartment for cells of the innate and the adaptive immune systems, including neutrophils, dendritic cells and monocytes, or B and T lymphocytes, respectively, which during infection will have been exposed to infectious agents in the tissue. For this reason whole blood from infected individuals provides an accessible source of clinically relevant material where an unbiased molecular phenotype can be obtained using gene expression microarrays as previously described for the study of cancer in tissues (Alizadeh A A., 2000; Golub, TR., 1999; Bittner, 2000), and autoimmunity (Bennet, 2003; Baechler, EC, 2003; Burczynski, ME, 2005; Chaussabel, D., 2005; Cobb, JP., 2005; Kaizer, EC., 2007; Allantaz, 2005; Allantaz, 2007), and inflammation (Thach, D C., 2005) and infectious disease (Ramillo, Blood, 2007) in blood or tissue (Bleharski, J R et al., 2003). Microarray analyses of gene expression in blood leucocytes have identified diagnostic and prognostic gene expression signatures, which have led to a better understanding of mechanisms of disease onset and responses to treatment (Bennet, L 2003; Rubins, KH., 2004; Baechler, EC, 2003; Pascual, V., 2005; Allantaz, F., 2007; Allantaz, F., 2007). These microarray approaches have been attempted for the study of active and latent TB but as yet have yielded small numbers of differentially expressed genes only (Jacobsen, M., Kaufmann, SH., 2006; Mistry, R, Lukey, PT, 2007), and in relatively small numbers of patients (Mistry, R., 2007), which may not be robust enough to distinguish between other inflammatory and infectious diseases.

Additional Methods.

Participant Recruitment and Patient Characterization. The local Research Ethics Committees at St. Mary's Hospital London, UK (REC 06/Q0403/128) and University of Cape Town, Cape Town, Republic of South Africa (REC 012/2007) approved the study. All participants were aged over 18 years old and gave written informed consent. Participants were recruited from St. Mary's Hospital and Hammersmith Hospital, Imperial College Healthcare NHS Trust, London, UK, Hillingdon Hospital, The Hillingdon Hospitals NHS Trust, Uxbridge, UK and the Ubuntu TB/HIV clinic, Khayelitsha, Cape Town, South Africa. Patients were prospectively recruited and sampled, before any anti-mycobacterial treatment was initiated, but only included in the final analysis if they met the full clinical criteria for their relevant study group. A subset of active TB patients recruited into the first cohort recruited in London was also sampled at 2 and 12 months after the initiation of therapy. Patients who were pregnant, immunosuppressed, or who had diabetes, or autoimmune disease were ineligible and excluded from this study. In South Africa, all participants had routine HIV testing using the Abbott Determine® HIV1/2 rapid antibody assay test kit (Abbott Laboratories, Abbott Park, Ill., USA). Active TB patients were confirmed by laboratory isolation of M. tuberculosis on mycobacterial culture of a respiratory specimen (either sputum or bronchoalvelolar lavage fluid) with sensitivity testing performed by The Royal Brompton Hospital Mycobacterial Reference Laboratory, London, UK or The Reference Lab of the National Health Laboratory Service, Groote Schuur Hospital, Cape Town. In the UK, latent TB patients were recruited from those referred to the TB clinic with a positive TST, together with a positive result using an IGRA. Latent TB participants in South Africa were recruited from individuals self-referring to the voluntary testing clinic at the Ubuntu TB/HIV clinic, and IGRA positivity alone was used to confirm the diagnosis, irrespective of TST result (although this was still performed). Healthy control participants were recruited from volunteers at the National Institute for Medical Research (NIMR), Mill Hill, London, UK. To meet the final criteria for study inclusion healthy volunteers had to be negative by both TST and IGRA.

Tuberculin Skin Testing. This was performed according to the UK guidelines¹ using 0.1 ml (2TU) tuberculin PPD (RT23, Serum Statens Institute, Copenhagen, Denmark). A positive TST was termed ≧6 mm if BCG unvaccinated, 15 mm if BCG vaccinated, as per the UK national guidelines².

Interferon Gamma Release Assay Testing. The QuantiFERON® Gold In-Tube assay (Cellestis, Carnegie, Australia) was performed according to the manufacturers instructions.

Total and Differential Leucocyte Counts. 2 mls of whole blood was collected into Terumo Venosafe 5 ml K2-EDTA tubes (Terumo Europe, Leuven, Belgium). Samples were then analysed within 4 hours using the Nihon Kohden MEK-6400 Automated Hematology Analyzer (Nihon Kohden Corporation, Tokyo, Japan).

Assessment of Radiographic Extent of Disease. Plain chest radiographs were obtained for all patients recruited in London as digital images and graded by three independent clinicians, blinded to the transcriptional profiles and the clinical data, using a modified version of the classification system of the U.S. National Tuberculosis and Respiratory Disease Association³. This system characterises the radiographic extent of disease into “Minimal”, “Moderately advanced” or “Far advanced” stages, according to criteria based upon the density and extent of lesions and presence of absence of cavitation. We modified the system for use in our study so that it also included a classification of “No disease, and accounted for the presence of pleural disease or lymphadenopathy. The system was then converted into a decision tree to aid classification (FIG. 9a).

RNA Sampling, Extraction and Processing for Microarray Analysis. 3 mls of whole blood was collected into Tempus tubes (Applied Biosystems, Foster City, Calif., USA), vigorously mixed immediately after collection, and stored between −20° C. and −80° C. before RNA extraction. RNA was isolated from Training Set samples using 1.5 mls whole blood and the PerfectPure RNA Blood kit (5 PRIME Inc, Gaithersburg, Md., USA). Test and Validation (SA) Set samples were extracted from 1 ml of whole blood using the MagMAX™-96 Blood RNA Isolation Kit (Applied Biosystems/Ambion, Austin, Tex., USA) according to the manufacturer's instructions. 2.5 mg of isolated total RNA was then globin reduced using the GLOBINclear™ 96-well format kit (Applied Biosystems/Ambion, Austin, Tex., USA) according to the manufacturer's instructions. Total and globin-reduced RNA integrity was assessed using an Agilent 2100 Bioanalyzer showing a quality of RIN of 7-9.5 (Agilent Technologies, Santa Clara, Calif., USA). RNA yield was assessed using a Nanodrop 1000 spectrophotometer (NanoDrop Products, The rmo Fisher Scientific Inc, Wilmington, Del., USA). Biotinylated, amplified antisense complementary RNA targets (cRNA) were then prepared from 200-250 ng of the globin-reduced RNA using the Illumina CustomPrep RNA amplification kit (Applied Biosystems/Ambion, Austin, Tex., USA). 750 ng of labelled cRNA was hybridized overnight to Illumina Human HT-12 BeadChip arrays (Illumina Inc, San Diego, Calif., USA), which contain more than 48,000 probes. The arrays were then washed, blocked, stained and scanned on an Illumina BeadStation 500 following the manufacturer's protocols. Illumina BeadStudio v2 software (Illumina Inc, San Diego, Calif., USA) was used to generate signal intensity values from the scans.

Separated cells isolation and RNA extraction. Whole blood was collected in EDTA. Neutrophils (CD15⁺), monocytes (CD14⁺), CD4⁺ T cells and CD8⁺ T cells were isolated sequentially using Dynabeads according to manufacturers instructions. RNA was extracted from whole blood (5′ Prime Perfect Pure kit) or separated cell populations (Qiagen RNEasy Mini Kit) and stored at −80° C. until use.

Microarray Data Analysis.

Normalisation.

Illumina BeadStudio v2 software was used to subtract background, and scale average signal intensity for each sample to the global average signal intensity for all samples. A gene expression analysis software program, GeneSpring GX, version 7.1.3 (Agilent Technologies, Santa Clara, Calif., USA, hereafter referred to as GeneSpring), was used to perform further normalisation. All signal intensity values less than 10 were set to equal 10. Next, per-gene normalisation was applied, by dividing the signal intensity of each probe in each sample by the median intensity for that probe across all samples. These normalised data were used for all downstream analyses except the assessment of molecular distance to health detailed below.

Class Prediction.

We utilised one of the class prediction tools available within GeneSpring. The prediction model employed the K-nearest neighbours algorithm, with 10 neighbours and a p value ratio cut off of 0.5. All genes from the 393 transcript list were used for the prediction. The prediction model was refined by cross-validation on the training set, with the one Active outlier excluded. This model was then used to predict the classification of the samples in the independent Test and Validation Sets. Where no prediction was made, this was recorded as an indeterminate result. Sensitivity, specificity and 95% confidence intervals (95% CI) were determined using GraphPad Prism version 5.02 for Windows. P-values were determined using two-sided Fisher's Exact test

Supervised analysis: (i) Transcriptional variance or “Molecular Distance to Health”. This technique was performed as previously described⁴. It aims to convert transcript abundance values into a representative score indicating the degree of transcriptional perturbation of a given sample compared to a healthy baseline. This is performed by determining whether the expression values of a given sample lie inside or outside two standard deviations from the mean of the healthy controls.

Supervised analysis: (ii) Pathway analysis. Additional functional analysis of differentially expressed genes was performed using Ingenuity Pathways Analysis (Ingenuity® Systems, Inc., Redwood, Calif., USA, www.ingenuity.com). Canonical pathways analysis identified the pathways from the Ingenuity Pathways Analysis that were most significantly represented in the dataset. The significance of the association between the dataset and the canonical pathway was measured using Fisher's Exact test to calculate a p-value representing the probability that the association between the transcripts in the dataset and the canonical pathway is explained by chance alone, with a Benjamini-Hochberg correction for multiple testing applied. The program can also be used to map the canonical network and overlay it with expression data from the dataset.

Supervised analysis: (iii) Transcriptional modular analysis. This analysis was performed as described previously^{4,5}. In the context of the present study, since the modular framework was derived using Affymetrix HG U133A&B GeneChips, it was necessary to translate the probes comprising the modules into their equivalents on the Illumina platform. RefSeq IDs were used to match probes between the Affymetrix HG U133 and Illumina WG-6 V2 platforms. Unambiguous matches were found for 2,109 out of the 5,348 Affymetrix probe sets, and these were used in the present modular analysis. The matching probes were preserved in their original modules. To graphically present the global transcriptional changes, for the disease group as a whole versus the healthy control group as a whole, spots are aligned on a grid, with each position corresponding to a different module based on their original definition. Spot intensity indicates the percentage of differentially expressed transcripts changing in the direction shown, from the total number of transcripts detected for that module, while spot colour indicates the polarity of the change (red=over-represented, blue=under-represented).

Multiplex Serum Protein Measurement. 1-4 ml blood was collected into serum clot activator tubes (either Greiner BioOne 1 ml vacuette tubes, ref 454098, Greiner BioOne, Kremsmünst, Austria; or BD 4 ml vacutainer tubes, ref 368975; Becton Dickinson). Tubes were centrifuged at 2000 g for 5 minutes at room temperature and the serum portion extracted and frozen at −80° C. pending analysis. Analysis was performed by multiplexed cytokine bead-based immunoassay by Millipore UK (Millipore UK Ltd, Dundee, UK) using the Milliplex® Multi-Analyte Profiling system (Millipore, Billerica, Mass., USA). The serum levels of 63 cytokines, chemokines, soluble receptors, growth factors, adhesion molecules and acute phase proteins were measured in this way in each sample. Samples were assayed for levels of MMP-9, C-reactive protein, serum amyloid A, EGF, Eotaxin, FGF-2, Flt-3 Ligand, Fractalkine, G-CSF, GM-CSF, GRO, IFN-α2, IFN-γ, IL-10, IL-12p40, IL-12p70, IL-13, IL-15, IL-17, IL-1α, IL-1β, IL-1Rα, IL-2, IL-4, IL-5, IL-6, IL-7, IL-8, IL-9, CXCL10 (IP10), MCP-1, MCP-3, MIP-1α, MIP-1β, PDGF-AA, PDGF-AB/BB, RANTES, soluble CD40 ligand, soluble IL-2RA, TGF-α, TNF-α, VEGF, MIF, soluble Fas, soluble Fas Ligand, tPAI-1, soluble ICAM-1, soluble VCAM-1, soluble CD30, soluble gp130, soluble IL-1RII, soluble IL-6R, soluble RAGE, soluble TNF-RI, soluble TNF-RII, IL-16, TGF-β1, TGF-β2 and TGFβ-3.

Flow Cytometry.

200 μl of whole blood (collected in Sodium-Heparin tubes) per staining panel was incubated with the appropriate antibodies for 20 minutes at room temperature in the dark. Red blood cells were then lysed using BD FACS lysing solution (BD Biosciences), incubating for 10 minutes at room temperature in the dark. Cells were spun down and washed in 2 ml FACS buffer (PBS/BSA/Azide) before being fixed in 1% paraformaldehyde. Samples were then run on a Beckman Coulter Cyan using Summit Software Version 3.02. Analysis was carried out using FlowJo Version 8.7.3 for Macintosh (Tree Star, Inc.). Gating strategies used are set out in FIGS. 11 and 12. Where appropriate pooled flow cytometry data was tested for significance using the Mann-Whitney Rank Sum U-test. All antibodies were purchased from BD Pharmingen or Caltag Laboratories (Invitrogen) except for CD45RA, which was purchased from Beckman Coulter.

Statistical Analysis.

Molecular distance to health and Modular Framework analysis calculations were performed using Microsoft Excel 2003 (Microsoft Corporation, Redmond, Wash., USA). Statistical analysis of continuous variables and correlation analysis was performed using GraphPad Prism version 5.02 for Windows (GraphPad Software, San Diego Calif. USA, www.graphpad.com). Analysis of categorical variables was performed using SPSS version 14 for Windows (Chicago, Ill., USA).

### REFERENCES FOR METHODS

1. Salisbury, D., Ramsay, M. Immunization against infectious diseases—the Green Book. D. O. Health, London The Stationery Office, 391-408 (2006).
2. National Institute for Health and Clinical Excellence. (Royal College of Physicians, UK, 2006).
3. Falk, A., O'Connor, J. B. Classification of pulmonary tuberculosis: Diagnosis standards and classification of tuberculosis. National tuberculosis and respiratory disease association 12, 68-76 (1969).
4. Pankla, R. et al. Genomic Transcriptional Profiling Identifies a Candidate Blood Biomarker Signature for the Diagnosis of Septicemic Melioidosis. Genome Biol In press (2009).
5. Chaussabel, D. et al. A modular analysis framework for blood genomics studies: application to systemic lupus erythematosus. Immunity 29, 150-64 (2008).

Genes in Module M1.3

| Relative | | | |
|---|---|---|---|
| normalised | | | |
| expression | Common Name | Gene Symbol | Description |
| 0.82 | FLJ31738; KIAA1209 | PLEKHG1 | pleckstrin homology domain containing, |
| | | | family G (with RhoGef domain) member 1 |
| 0.778 | SPI-B | SPIB | Spi-B transcription factor (Spi-1/PU.1 |
| | | | related) |
| 0.767 | EVI9; CTIP1; BCL11A-L; | BCL11A | B-cell CLL/lymphoma 11A (zinc finger |
| | BCL11A-S; FLJ10173; | | protein) |
| | FLJ34997; KIAA1809; BCL11A- | | |
| | XL | | |
| 0.715 | MGC20446 | CYBASC3 | cytochrome b, ascorbate dependent 3 |
| 0.677 | NIDD; MGC42530 | ZDHHC23 | zinc finger, DHHC-type containing 23 |
| 0.629 | ESG; ESG1; GRG1 | TLE1 | transducin-like enhancer of split 1 (E(sp1) |
| | | | homolog, Drosophila) |
| 0.612 | B29; IGB | CD79B | CD79b molecule, immunoglobulin- |
| | | | associated beta |
| 0.581 | LYB2; CD72b | CD72 | CD72 molecule |
| 0.559 | KIAA0977 | COBLL1 | COBL-like 1 |
| 0.556 | BASH; Ly57; SLP65; BLNK-s; | BLNK | B-cell linker |
| | SLP-65; MGC111051 | | |
| 0.543 | TCL1 | TCL1A | T-cell leukemia/lymphoma 1A |
| 0.518 | c-Myc | MYC | v-myc myelocytomatosis viral oncogene |
| | | | homolog (avian) |
| 0.512 | BANK; FLJ20706; FLJ34204 | BANK1 | B-cell scaffold protein with ankyrin repeats 1 |
| 0.51 | B4; MGC12802 | CD19 | CD19 molecule |
| 0.496 | FCRH1; IFGP1; IRTA5; RP11- | FCRL1 | Fc receptor-like 1 |
| | 367J7.7; DKFZp667O1421 | | |
| 0.487 | FLJ00058 | GNG7 | guanine nucleotide binding protein (G |
| | | | protein), gamma 7 |
| 0.482 | FLJ21562; FLJ43762 | C13orf18 | chromosome 13 open reading frame 18 |
| 0.477 | BRDG1; STAP1 | BRDG1 | BCR downstream signaling 1 |
| 0.471 | MGC10442 | BLK | B lymphoid tyrosine kinase |
| 0.467 | R1; JPO2; RAM2; | CDCA7L | cell division cycle associated 7-like |
| | DKFZp762L0311 | | |
| 0.445 | ORP10; OSBP9; FLJ20363 | OSBPL10 | oxysterol binding protein-like 10 |
| 0.397 | 8HS20; N27C7-2 | VPREB3 | pre-B lymphocyte gene 3 |
| 0.361 | LAF4; MLLT2-like | AFF3 | AF4/FMR2 family, member 3 |
| 0.334 | FCRL; FREB; FCRLX; FCRLb; | FCRLM1 | Fc receptor-like A |
| | FCRLd; FCRLe; FCRLM1; | | |
| | FCRLc1; FCRLc2; MGC4595; | | |
| | RP11-474I16.5 | | |

Genes in Module M2.8

| Relative | | | |
|---|---|---|---|
| normalised | | | |
| expression | Common Name | Gene Symbol | Description |
| 0.871 | KPL1; PHR1; PHRET1 | PLEKHB1 | pleckstrin homology domain containing, |
| | | | family B (evectins) member 1 |
| 0.816 | MGC132014 | INPP4B | inositol polyphosphate-4-phosphatase, type |
| | | | II, 105 kDa |
| 0.732 | SEP2; SEPT2; KIAA0128; | 6-Sep | septin 6 |
| | MGC16619; MGC20339; RP5- | | |
| | 876A24.2 | | |
| 0.711 | GIL | AQP3 | aquaporin 3 (Gill blood group) |
| 0.691 | FLJ36386 | LZTFL1 | leucine zipper transcription factor-like 1 |
| 0.67 | p52; p75; PAIP; DFS70; | PSIP1 | PC4 and SFRS1 interacting protein 1 |
| | LEDGF; PSIP2; MGC74712 | | |
| 0.669 | GRG; ESP1; GRG5; TLE5; | AES | amino-terminal enhancer of split |
| | AES-1; AES-2 | | |
| 0.668 | p33; TNFC; TNFSF3 | LTB | lymphotoxin beta (TNF superfamily, |
| | | | member 3) |
| 0.646 | KIAA0521; MGC15913 | ARHGEF18 | rho/rac guanine nucleotide exchange factor |
| | | | (GEF) 18 |
| 0.634 | TEM3; TEM7; FLJ36270; | PLXDC1 | plexin domain containing 1 |
| | FLJ45632; DKFZp686F0937 | | |
| 0.626 | HPIP | PBXIP1 | pre-B-cell leukemia homeobox interacting |
| | | | protein 1 |
| 0.621 | KIAA0495; MGC138189 | KIAA0495 | KIAA0495 |
| 0.615 | KUP; ZNF46 | ZBTB25 | zinc finger and BTB domain containing 25 |
| 0.61 | FLJ20729; FLJ20760; NY-BR- | C1orf181 | chromosome 1 open reading frame 181 |
| | 75; MGC131963 | | |
| 0.609 | AAG6; PKCA; PRKACA; | PRKCA | protein kinase C, alpha |
| | MGC129900; MGC129901; | | |
| | PKC-alpha | | |
| 0.604 | CGI-25 | NOSIP | nitric oxide synthase interacting protein |
| 0.602 | FLJ20152; FLJ22155; | FLJ20152 | family with sequence similarity 134, |
| | FLJ22179 | | member B |
| 0.599 | FRA3B; AP3Aase | FHIT | fragile histidine triad gene |
| 0.596 | WDR74 | WDR74 | WD repeat domain 74; synonyms: |
| | | | FLJ10439, FLJ21730; Homo sapiens WD |
| | | | repeat domain 74 (WDR74), mRNA. |
| 0.595 | E25A; BRICD2A | ITM2A | integral membrane protein 2A |
| 0.587 | HPF2 | ZNF84 | zinc finger protein 84 |
| 0.58 | SEK; HEK8; TYRO1 | EPHA4 | EPH receptor A4 |
| 0.578 | SID1; SID-1; FLJ20174; | SIDT1 | SID1 transmembrane family, member 1 |
| | B830021E24Rik | | |
| 0.557 | LTBP2; LTBP-3; pp6425; | LTBP3 | latent transforming growth factor beta |
| | FLJ33431; FLJ39893; | | binding protein 3 |
| | FLJ42533; FLJ44138; | | |
| | DKFZP586M2123 | | |
| 0.556 | V; RASGRP; hRasGRP1; | RASGRP1 | RAS guanyl releasing protein 1 (calcium |
| | MGC129998; MGC129999; | | and DAG-regulated) |
| | CALDAG-GEFI; CALDAG- | | |
| | GEFII | | |
| 0.546 | TTF; ARHH | RHOH | ras homolog gene family, member H |
| 0.545 | LAT3; LAT-2; y+LAT-2; | SLC7A6 | solute carrier family 7 (cationic amino acid |
| | KIAA0245; DKFZp686K15246 | | transporter, y+ system), member 6 |
| 0.541 | TP120 | CD6 | CD6 molecule |
| 0.537 | MGC29816 | CHMP7 | CHMP family, member 7 |
| 0.53 | DAGK; DAGK1; MGC12821; | DGKA | diacylglycerol kinase, alpha 80 kDa |
| | MGC42356; DGK-alpha | | |
| 0.523 | hly9; mLY9; CD229; SLAMF3 | LY9 | lymphocyte antigen 9 |
| 0.52 | EMT; LYK; PSCTK2; | ITK | IL2-inducible T-cell kinase |
| | MGC126257; MGC126258 | | |
| 0.519 | TACTILE; MGC22596; | CD96 | CD96 molecule |
| | DKFZp667E2122 | | |
| 0.518 | SEP2; SEPT2; KIAA0128; | 6-Sep | septin 6 |
| | MGC16619; MGC20339; RP5- | | |
| | 876A24.2 | | |
| 0.501 | SCAP1; SKAP55 | SCAP1 | src kinase associated phosphoprotein 1 |
| 0.49 | FLJ12884; MGC130014; | C10orf38 | chromosome 10 open reading frame 38 |
| | MGC130015 | | |
| 0.488 | T1; LEU1 | CD5 | CD5 molecule |
| 0.487 | MAL | MAL | mal, T-cell differentiation protein |
| 0.484 | SATB1 | SATB1 | SATB homeobox 1 |
| 0.48 | LDH-H; TRG-5 | LDHB | lactate dehydrogenase B |
| 0.473 | Ray; FLJ39121; | SH3YL1 | SH3 domain containing, Ysc84-like 1 (S. cerevisiae) |
| | DKFZP586F1318 | | |
| 0.466 | P19; SGRF; IL-23; IL-23A; | IL23A | interleukin 23, alpha subunit p19 |
| | IL23P19; MGC79388 | | |
| 0.465 | KE6; FABG; HKE6; FABGL; | HSD17B8 | hydroxysteroid (17-beta) dehydrogenase 8 |
| | RING2; H2-KE6; D6S2245E; | | |
| | dJ1033B10.9 | | |
| 0.456 | ARH; ARH1; ARH2; FHCB1; | LDLRAP1 | low density lipoprotein receptor adaptor |
| | FHCB2; MGC34705; | | protein 1 |
| | DKFZp586D0624 | | |
| 0.453 | MGC45416; | OCIAD2 | OCIA domain containing 2 |
| | DKFZp686C03164 | | |
| 0.451 | CD172g; SIRPB2; SIRP-B2; | SIRPB2 | signal-regulatory protein gamma |
| | bA77C3.1; SIRPgamma | | |
| 0.435 | GP40; TP41; Tp40; LEU-9 | CD7 | CD7 molecule |
| 0.427 | MGC15763 | MGC15763 | oxidoreductase NAD-binding domain |
| | | | containing 1 |
| 0.41 | AS160; DKFZp779C0666 | TBC1D4 | TBC1 domain family, member 4 |
| 0.404 | HMIC; MAN1C; MAN1A3; | MAN1C1 | mannosidase, alpha, class 1C, member 1 |
| | pp6318 | | |
| 0.401 | Tp44; MGC138290 | CD28 | CD28 molecule |
| 0.394 | FLJ12586 | ZNF329 | zinc finger protein 329 |
| 0.39 | TCF-1; MGC47735 | TCF7 | transcription factor 7 (T-cell specific, HMG- |
| | | | box) |
| 0.385 | ABLIM; LIMAB1; LIMATIN; | ABLIM1 | actin binding LIM protein 1 |
| | MGC1224; FLJ14564; | | |
| | KIAA0059; DKFZp781D0148 | | |
| 0.383 | NSE2; BCMP101 | FAM84B | family with sequence similarity 84, member B |
| 0.377 | TOSO | FAIM3 | Fas apoptotic inhibitory molecule 3 |
| 0.371 | EEIG1; C9orf132; MGC50853; | C9orf132 | family with sequence similarity 102, |
| | bA203J24.7 | | member A |
| 0.36 | RIT1; CTIP2; CTIP-2; hRIT1- | BCL11B | B-cell CLL/lymphoma 11B (zinc finger |
| | alpha | | protein) |
| 0.33 | CLP24; FLJ20898; | C16orf30 | chromosome 16 open reading frame 30 |
| | MGC111564 | | |
| 0.315 | TCF1ALPHA; | LEF1 | lymphoid enhancer-binding factor 1 |
| | DKFZp586H0919 | | |
| 0.29 | BLR2; EBI1; CD197; | CCR7 | chemokine (C-C motif) receptor 7 |
| | CDw197; CMKBR7 | | |
| 0.244 | STK37; PASKIN; KIAA0135; | PASK | PAS domain containing serine/threonine |
| | DKFZP434O051; | | kinase |
| | DKFZp686P2031 | | |
| 0.205 | NRP2 | NELL2 | NEL-like 2 (chicken) |

Genes in Modules M1.5

| Relative | | | |
|---|---|---|---|
| normalised | | | |
| expression | Common Name | Gene Symbol | Description |
| 2.384 | VHR | DUSP3 | dual specificity phosphatase 3 (vaccinia |
| | | | virus phosphatase VH1-related) |
| 2.139 | 4.1B; DAL1; DAL-1; | EPB41L3 | erythrocyte membrane protein band 4.1-like 3 |
| | FLJ37633; KIAA0987 | | |
| 2.014 | HXK3; HKIII | HK3 | hexokinase 3 (white cell) |
| 1.972 | HL14; MGC75071 | LGALS2 | lectin, galactoside-binding, soluble, 2 |
| 1.844 | KYNU | KYNU | kynureninase (L-kynurenine hydrolase) |
| 1.618 | BLVR; BVRA | BLVRA | biliverdin reductase A |
| 1.594 | RP35; SEMB; SEMAB; | SEMA4A | sema domain, immunoglobulin domain (Ig), |
| | CORD10; FLJ12287; RP11- | | transmembrane domain (TM) and short |
| | 54H19.2 | | cytoplasmic domain, (semaphorin) 4A |
| 1.535 | | GRN | |
| 1.531 | G6S; MGC21274 | GNS | glucosamine (N-acetyl)-6-sulfatase |
| | | | (Sanfilippo disease IIID) |
| 1.524 | FOAP-10; EMILIN-2; | EMILIN2 | elastin microfibril interfacer 2 |
| | FLJ33200 | | |
| 1.507 | cent-b; HSA272195 | CENTA2 | centaurin, alpha 2 |
| 1.449 | APPS; CPSB | CTSB | cathepsin B |
| 1.438 | ASGPR; CLEC4H1; Hs.12056 | ASGR1 | asialoglycoprotein receptor 1 |
| 1.433 | CD32; FCG2; FcGR; CD32A; | FCGR2A | Fc fragment of IgG, low affinity IIa, |
| | CDw32; FCGR2; IGFR2; | | receptor (CD32) |
| | FCGR2A1; MGC23887; | | |
| | MGC30032 | | |
| 1.425 | TIL4; CD282 | TLR2 | toll-like receptor 2 |
| 1.424 | PI; A1A; AAT; PI1; A1AT; | SERPINA1 | serpin peptidase inhibitor, clade A (alpha-1 |
| | MGC9222; PRO2275; | | antiproteinase, antitrypsin), member 1 |
| | MGC23330 | | |
| 1.413 | TEM7R; FLJ14623 | PLXDC2 | plexin domain containing 2 |
| 1.41 | CD14 | CD14 | CD14 molecule |
| 1.398 | Rab22B | RAB31 | RAB31, member RAS oncogene family |
| 1.386 | FEX1; FEEL-1; FELE-1; | STAB1 | stabilin 1 |
| | STAB-1; CLEVER-1; | | |
| | KIAA0246 | | |
| 1.352 | MYD88 | MYD88 | myeloid differentiation primary response |
| | | | gene (88) |
| 1.349 | MLN70; S100C | S100A11 | S100 calcium binding protein A11 |
| 1.347 | FLJ22662 | FLJ22662 | hypothetical protein FLJ22662 |
| 1.346 | CLN2; GIG1; LPIC; TPP I; | TPP1 | tripeptidyl peptidase I |
| | MGC21297 | | |
| 1.251 | p75; TBPII; TNFBR; TNFR2; | TNFRSF1B | tumor necrosis factor receptor superfamily, |
| | CD120b; TNFR80; TNF-R75; | | member 1B |
| | p75TNFR; TNF-R-II | | |
| 1.239 | JTK9 | HCK | hemopoietic cell kinase |
| 1.172 | IBA1; AIF-1; IRT-1 | AIF1 | allograft inflammatory factor 1 |

Genes in Modules M2.6

| Relative | | | |
|---|---|---|---|
| normalised | | | |
| expression | Common Name | Gene Symbol | Description |
| 2.409 | HsT287 | ZNF516 | zinc finger protein 516 |
| 2.286 | CRISP11; LCRISP2; | CRISPLD2 | cysteine-rich secretory protein LCCL |
| | MGC74865; DKFZP434B044 | | domain containing 2 |
| 2.177 | MAG1; GPAT3; AGPAT8; | HMFN0839 | lung cancer metastasis-associated protein |
| | MGC11324 | | |
| 2.095 | CDD | CDA | cytidine deaminase |
| 2.094 | CRBP4; CRBPIV; MGC70641 | RBP7 | retinol binding protein 7, cellular |
| 1.917 | SSC1; HsT17287 | AQP9 | aquaporin 9 |
| 1.916 | GMR; CD116; CSF2R; | CSF2RA | colony stimulating factor 2 receptor, alpha, |
| | CDw116; CSF2RX; CSF2RY; | | low-affinity (granulocyte-macrophage) |
| | GMCSFR; CSF2RAX; | | |
| | CSF2RAY; MGC3848; | | |
| | MGC4838; GM-CSF-R-alpha | | |
| 1.853 | G0S8 | RGS2 | regulator of G-protein signalling 2, 24 kDa |
| 1.734 | HKII; HXK2; | HK2 | hexokinase 2 |
| | DKFZp686M1669 | | |
| 1.734 | BB1 | LENG4 | leukocyte receptor cluster (LRC) member 4 |
| 1.701 | UB1; CEP3; BORG2; | CDC42EP3 | CDC42 effector protein (Rho GTPase |
| | FLJ46903 | | binding) 3 |
| 1.671 | SPAL2; FLJ23126; FLJ23632; | SIPA1L2 | signal-induced proliferation-associated 1 |
| | KIAA1389 | | like 2 |
| 1.669 | ST1; SYCL; MDA-9; TACIP18 | SDCBP | syndecan binding protein (syntenin) |
| 1.669 | CAN; CAIN; N214; D9S46E; | NUP214 | nucleoporin 214 kDa |
| | MGC104525 | | |
| 1.651 | | SLC19A1 | |
| 1.65 | LPB3; S1P3; EDG-3; S1PR3; | EDG3 | endothelial differentiation, sphingolipid G- |
| | FLJ37523; MGC71696 | | protein-coupled receptor, 3 |
| 1.642 | FPR; FMLP | FPR1 | formyl peptide receptor 1 |
| 1.61 | GPCR1; GPR86; GPR94; | P2RY13 | purinergic receptor P2Y, G-protein coupled, |
| | P2Y13; SP174; FKSG77 | | 13 |
| 1.606 | WDR80; FLJ00012 | ATG16L2 | ATG16 autophagy related 16-like 2 (S. cerevisiae) |
| 1.601 | LENG5; SEN34; SEN34L | TSEN34 | tRNA splicing endonuclease 34 homolog (S. cerevisiae) |
| 1.575 | FPF; p55; p60; TBP1; TNF-R; | TNFRSF1A | tumor necrosis factor receptor superfamily, |
| | TNFAR; TNFR1; p55-R; | | member 1A |
| | CD120a; TNFR55; TNFR60; | | |
| | TNF-R-I; TNF-R55; | | |
| | MGC19588 | | |
| 1.572 | PELI2 | PELI2 | pellino homolog 2 (Drosophila) |
| 1.562 | FLJ13052; FLJ37724; | NADK | NAD kinase |
| | dJ283E3.1; RP1-283E3.6 | | |
| 1.558 | 5-LO; 5LPG; LOG5; | ALOX5 | arachidonate 5-lipoxygenase |
| | MGC163204 | | |
| 1.534 | TMPIT | TMPIT | transmembrane protein induced by tumor |
| | | | necrosis factor alpha |
| 1.517 | FLJ31978 | GLT1D1 | glycosyltransferase 1 domain containing 1 |
| 1.517 | PFKFB4 | PFKFB4 | 6-phosphofructo-2-kinase/fructose-2,6- |
| | | | biphosphatase 4 |
| 1.516 | FLJ22470; KIAA1993; | ZBTB34 | zinc finger and BTB domain containing 34 |
| | MGC24652; RP11-106H5.1 | | |
| 1.482 | P39; VATX; VMA6; ATP6D; | ATP6V0D1 | ATPase, H+ transporting, lysosomal 38 kDa, |
| | ATP6DV; VPATPD | | V0 subunit d1 |
| 1.473 | PRAM-1; MGC39864 | PRAM1 | PML-RARA regulated adaptor molecule 1 |
| 1.471 | BIT; MFR; P84; SIRP; MYD- | PTPNS1 | signal-regulatory protein alpha |
| | 1; SHPS1; CD172A; PTPNS1; | | |
| | SHPS-1; SIRPalpha; | | |
| | SIRPalpha2; SIRP-ALPHA-1 | | |
| 1.463 | M130; MM130 | CD163 | CD163 molecule |
| 1.434 | AF-1; IFGR2; IFNGT1 | IFNGR2 | interferon gamma receptor 2 (interferon |
| | | | gamma transducer 1) |
| 1.405 | RALB | RALB | v-ral simian leukemia viral oncogene |
| | | | homolog B (ras related; GTP binding |
| | | | protein) |
| 1.405 | SLCO3A1 | SLCO3A1 | solute carrier organic anion transporter |
| | | | family, member 3A1; synonyms: OATP-D, |
| | | | OATP3A1, FLJ40478, SLC21A11; solute |
| | | | carrier family 21 (organic anion |
| | | | transporter), member 11; Homo sapiens |
| | | | solute carrier organic anion transporter |
| | | | family, member 3A1 (SLCO3A1), mRNA. |
| 1.397 | PTPE; HPTPE; | PTPRE | protein tyrosine phosphatase, receptor type, E |
| | DKFZp313F1310; R-PTP- | | |
| | EPSILON | | |
| 1.397 | RCC4; FLJ14784 | DIRC2 | disrupted in renal carcinoma 2 |
| 1.396 | DAP12; KARAP; PLOSL | TYROBP | TYRO protein tyrosine kinase binding |
| | | | protein |
| 1.371 | B144; LST-1; D6S49E; | LST1 | leukocyte specific transcript 1 |
| | MGC119006; MGC119007 | | |
| 1.359 | BFD; PFC; PFD; PROPERDIN | PFC | complement factor properdin |
| 1.31 | CAG4A; ERDA5; PRAT4A | TNRC5 | trinucleotide repeat containing 5 |
| 1.307 | CD18; TNFCR; D12S370; | LTBR | lymphotoxin beta receptor (TNFR |
| | TNFR-RP; TNFRSF3; TNFR2- | | superfamily, member 3) |
| | RP; LT-BETA-R; TNF-R-III | | |
| 1.305 | CEB | VAMP3 | vesicle-associated membrane protein 3 |
| | | | (cellubrevin) |
| 1.304 | CSC-21K | TIMP2 | TIMP metallopeptidase inhibitor 2 |
| 1.301 | BPOZ; EF1ABP; PP2259; | ABTB1 | ankyrin repeat and BTB (POZ) domain |
| | MGC20585 | | containing 1 |
| 1.294 | C6orf209; FLJ11240; | LMBRD1 | LMBR1 domain containing 1 |
| | bA810I22.1; RP11-810I22.1 | | |
| 1.266 | PBF; C21orf1; C21orf3 | PTTG1IP | pituitary tumor-transforming 1 interacting |
| | | | protein |
| 1.235 | ZFYVE10; FLJ32333; | MTMR3 | myotubularin related protein 3 |
| | KIAA0371; FYVE-DSP1 | | |
| 1.216 | CFP1; CBCP1; C10orf9 | C10orf9 | cyclin Y |
| 1.2 | SPT4H; SUPT4H | SUPT4H1 | suppressor of Ty 4 homolog 1 (S. cerevisiae) |

Genes in Module M2.2

| Relative | | | |
|---|---|---|---|
| normalised | | | |
| expression | Common Name | Gene Symbol | Description |
| 2.409 | HsT287 | ZNF516 | zinc finger protein 516 |
| 2.286 | CRISP11; LCRISP2; | CRISPLD2 | cysteine-rich secretory protein LCCL |
| | MGC74865; DKFZP434B044 | | domain containing 2 |
| 2.177 | MAG1; GPAT3; AGPAT8; | HMFN0839 | lung cancer metastasis-associated protein |
| | MGC11324 | | |
| 2.095 | CDD | CDA | cytidine deaminase |
| 2.094 | CRBP4; CRBPIV; MGC70641 | RBP7 | retinol binding protein 7, cellular |
| 1.917 | SSC1; HsT17287 | AQP9 | aquaporin 9 |
| 1.916 | GMR; CD116; CSF2R; | CSF2RA | colony stimulating factor 2 receptor, alpha, |
| | CDw116; CSF2RX; CSF2RY; | | low-affinity (granulocyte-macrophage) |
| | GMCSFR; CSF2RAX; | | |
| | CSF2RAY; MGC3848; | | |
| | MGC4838; GM-CSF-R-alpha | | |
| 1.853 | G0S8 | RGS2 | regulator of G-protein signalling 2, 24 kDa |
| 1.734 | HKII; HXK2; | HK2 | hexokinase 2 |
| | DKFZp686M1669 | | |
| 1.734 | BB1 | LENG4 | leukocyte receptor cluster (LRC) member 4 |
| 1.701 | UB1; CEP3; BORG2; | CDC42EP3 | CDC42 effector protein (Rho GTPase |
| | FLJ46903 | | binding) 3 |
| 1.671 | SPAL2; FLJ23126; FLJ23632; | SIPA1L2 | signal-induced proliferation-associated 1 |
| | KIAA1389 | | like 2 |
| 1.669 | ST1; SYCL; MDA-9; TACIP18 | SDCBP | syndecan binding protein (syntenin) |
| 1.669 | CAN; CAIN; N214; D9S46E; | NUP214 | nucleoporin 214 kDa |
| | MGC104525 | | |
| 1.651 | | SLC19A1 | |
| 1.65 | LPB3; S1P3; EDG-3; S1PR3; | EDG3 | endothelial differentiation, sphingolipid G- |
| | FLJ37523; MGC71696 | | protein-coupled receptor, 3 |
| 1.642 | FPR; FMLP | FPR1 | formyl peptide receptor 1 |
| 1.61 | GPCR1; GPR86; GPR94; | P2RY13 | purinergic receptor P2Y, G-protein coupled, |
| | P2Y13; SP174; FKSG77 | | 13 |
| 1.606 | WDR80; FLJ00012 | ATG16L2 | ATG16 autophagy related 16-like 2 (S. cerevisiae) |
| 1.601 | LENG5; SEN34; SEN34L | TSEN34 | tRNA splicing endonuclease 34 homolog (S. cerevisiae) |
| 1.575 | FPF; p55; p60; TBP1; TNF-R; | TNFRSF1A | tumor necrosis factor receptor superfamily, |
| | TNFAR; TNFR1; p55-R; | | member 1A |
| | CD120a; TNFR55; TNFR60; | | |
| | TNF-R-I; TNF-R55; | | |
| | MGC19588 | | |
| 1.572 | PELI2 | PELI2 | pellino homolog 2 (Drosophila) |
| 1.562 | FLJ13052; FLJ37724; | NADK | NAD kinase |
| | dJ283E3.1; RP1-283E3.6 | | |
| 1.558 | 5-LO; 5LPG; LOG5; | ALOX5 | arachidonate 5-lipoxygenase |
| | MGC163204 | | |
| 1.534 | TMPIT | TMPIT | transmembrane protein induced by tumor |
| | | | necrosis factor alpha |
| 1.517 | FLJ31978 | GLT1D1 | glycosyltransferase 1 domain containing 1 |
| 1.517 | PFKFB4 | PFKFB4 | 6-phosphofructo-2-kinase/fructose-2,6- |
| | | | biphosphatase 4 |
| 1.516 | FLJ22470; KIAA1993; | ZBTB34 | zinc finger and BTB domain containing 34 |
| | MGC24652; RP11-106H5.1 | | |
| 1.482 | P39; VATX; VMA6; ATP6D; | ATP6V0D1 | ATPase, H+ transporting, lysosomal 38 kDa, |
| | ATP6DV; VPATPD | | V0 subunit d1 |
| 1.473 | PRAM-1; MGC39864 | PRAM1 | PML-RARA regulated adaptor molecule 1 |
| 1.471 | BIT; MFR; P84; SIRP; MYD- | PTPNS1 | signal-regulatory protein alpha |
| | 1; SHPS1; CD172A; PTPNS1; | | |
| | SHPS-1; SIRPalpha; | | |
| | SIRPalpha2; SIRP-ALPHA-1 | | |
| 1.463 | M130; MM130 | CD163 | CD163 molecule |
| 1.434 | AF-1; IFGR2; IFNGT1 | IFNGR2 | interferon gamma receptor 2 (interferon |
| | | | gamma transducer 1) |
| 1.405 | RALB | RALB | v-ral simian leukemia viral oncogene |
| | | | homolog B (ras related; GTP binding |
| | | | protein) |
| 1.405 | SLCO3A1 | SLCO3A1 | solute carrier organic anion transporter |
| | | | family, member 3A1; synonyms: OATP-D, |
| | | | OATP3A1, FLJ40478, SLC21A11; solute |
| | | | carrier family 21 (organic anion |
| | | | transporter), member 11; Homo sapiens |
| | | | solute carrier organic anion transporter |
| | | | family, member 3A1 (SLCO3A1), mRNA. |
| 1.397 | PTPE; HPTPE; | PTPRE | protein tyrosine phosphatase, receptor type, E |
| | DKFZp313F1310; R-PTP- | | |
| | EPSILON | | |
| 1.397 | RCC4; FLJ14784 | DIRC2 | disrupted in renal carcinoma 2 |
| 1.396 | DAP12; KARAP; PLOSL | TYROBP | TYRO protein tyrosine kinase binding |
| | | | protein |
| 1.371 | B144; LST-1; D6S49E; | LST1 | leukocyte specific transcript 1 |
| | MGC119006; MGC119007 | | |
| 1.359 | BFD; PFC; PFD; PROPERDIN | PFC | complement factor properdin |
| 1.31 | CAG4A; ERDA5; PRAT4A | TNRC5 | trinucleotide repeat containing 5 |
| 1.307 | CD18; TNFCR; D12S370; | LTBR | lymphotoxin beta receptor (TNFR |
| | TNFR-RP; TNFRSF3; TNFR2- | | superfamily, member 3) |
| | RP; LT-BETA-R; TNF-R-III | | |
| 1.305 | CEB | VAMP3 | vesicle-associated membrane protein 3 |
| | | | (cellubrevin) |
| 1.304 | CSC-21K | TIMP2 | TIMP metallopeptidase inhibitor 2 |
| 1.301 | BPOZ; EF1ABP; PP2259; | ABTB1 | ankyrin repeat and BTB (POZ) domain |
| | MGC20585 | | containing 1 |
| 1.294 | C6orf209; FLJ11240; | LMBRD1 | LMBR1 domain containing 1 |
| | bA810I22.1; RP11-810I22.1 | | |
| 1.266 | PBF; C21orf1; C21orf3 | PTTG1IP | pituitary tumor-transforming 1 interacting |
| | | | protein |
| 1.235 | ZFYVE10; FLJ32333; | MTMR3 | myotubularin related protein 3 |
| | KIAA0371; FYVE-DSP1 | | |
| 1.216 | CFP1; CBCP1; C10orf9 | C10orf9 | cyclin Y |
| 1.2 | SPT4H; SUPT4H | SUPT4H1 | suppressor of Ty 4 homolog 1 (S. cerevisiae) |

Genes in Module 3.1

| Relative | | | |
|---|---|---|---|
| normalised | | | |
| expression | Common Name | Gene Symbol | Description |
| 17.93 | MGC22805 | ANKRD22 | ankyrin repeat domain 22 |
| 14.86 | C1IN; C1NH; HAE1; HAE2; | SERPING1 | serpin peptidase inhibitor, clade G (C1 |
| | C1INH | | inhibitor), member 1, (angioedema, |
| | | | hereditary) |
| 9.425 | cig5; vig1; 2510004L01Rik | RSAD2 | radical S-adenosyl methionine domain |
| | | | containing 2 |
| 8.938 | BRESI1; MGC29634 | EPSTI1 | epithelial stromal interaction 1 (breast) |
| 8.226 | GS3686; C1orf29 | IFI44L | interferon-induced protein 44-like |
| 7.566 | GBP1 | GBP1 | guanylate binding protein 1, interferon- |
| | | | inducible, 67 kDa |
| 5.677 | p44; MTAP44 | IFI44 | interferon-induced protein 44 |
| 4.701 | LAP; PEPS; LAPEP | LAP3 | leucine aminopeptidase 3 |
| 4.401 | IRG2; IFI60; IFIT4; ISG60; | IFIT3 | interferon-induced protein with |
| | RIG-G; CIG-49; GARG-49 | | tetratricopeptide repeats 3 |
| 4.091 | OIAS; IFI-4; OIASI | OAS1 | 2′,5′-oligoadenylate synthetase 1, 40/46 kDa |
| 3.947 | p100; MGC133260 | OAS3 | 2′-5′-oligoadenylate synthetase 3, 100 kDa |
| 3.944 | G1P2; UCRP; IFI15 | G1P2 | ISG15 ubiquitin-like modifier |
| 3.915 | UEF1; DRIF2; C7orf6; | SAMD9L | sterile alpha motif domain containing 9-like |
| | FLJ39885; KIAA2005 | | |
| 3.909 | MMTRA1B | PLSCR1 | phospholipid scramblase 1 |
| 3.792 | XAF1; BIRC4BP; | BIRC4BP | XIAP associated factor-1 |
| | HSXIAPAF1 | | |
| 3.731 | RIGE; SCA2; RIG-E; SCA-2; | LY6E | lymphocyte antigen 6 complex, locus E |
| | TSA-1 | | |
| 3.726 | C7; IFI10; INP10; IP-10; crg-2; | CXCL10 | chemokine (C—X—C motif) ligand 10 |
| | mob-1; SCYB10; gIP-10 | | |
| 3.668 | FBG2; FBS2; FBX6; Fbx6b | FBXO6 | F-box protein 6 |
| 3.652 | RNF94; STAF50; GPSTAF50 | TRIM22 | tripartite motif-containing 22 |
| 3.619 | LOC129607 | LOC129607 | hypothetical protein LOC129607 |
| 3.419 | ISGF-3; STAT91; | STAT1 | signal transducer and activator of |
| | DKFZp686B04100 | | transcription 1, 91 kDa |
| 3.398 | TRIP14; p59OASL | OASL | 2′-5′-oligoadenylate synthetase-like |
| 3.284 | IFP35; FLJ21753 | IFI35 | interferon-induced protein 35 |
| 3.154 | LOC26010; DNAPTP6; | DNAPTP6 | viral DNA polymerase-transactivated |
| | DKFZp564A2416 | | protein 6 |
| 3.076 | BAL; BAL1; FLJ26637; | PARP9 | poly (ADP-ribose) polymerase family, |
| | FLJ41418; MGC: 7868; | | member 9 |
| | DKFZp666B0810; | | |
| | DKFZp686M15238 | | |
| 3.032 | BAL2; KIAA1268 | PARP14 | poly (ADP-ribose) polymerase family, |
| | | | member 14 |
| 2.977 | RIG-B; UBCH8; MGC40331 | UBE2L6 | ubiquitin-conjugating enzyme E2L 6 |
| 2.839 | APT1; PSF1; ABC17; ABCB2; | TAP1 | transporter 1, ATP-binding cassette, sub- |
| | RING4; TAP1N; D6S114E; | | family B (MDR/TAP) |
| | FLJ26666; FLJ41500; | | |
| | TAP1*0102N | | |
| 2.814 | MX; MxA; IFI78; IFI-78K | MX1 | myxovirus (influenza virus) resistance 1, |
| | | | interferon-inducible protein p78 (mouse) |
| 2.632 | | IRF7 | |
| 2.511 | GCH; DYT5; GTPCH1; GTP- | GCH1 | GTP cyclohydrolase 1 (dopa-responsive |
| | CH-1 | | dystonia) |
| 2.434 | 9-27; CD225; IFI17; LEU13 | IFITM1 | interferon induced transmembrane protein 1 |
| | | | (9-27) |
| 2.415 | G10P2; IFI54; ISG54; cig42; | IFIT2 | interferon-induced protein with |
| | IFI-54; GARG-39; ISG-54K | | tetratricopeptide repeats 2 |
| 2.414 | Hlcd; MDA5; MDA-5; | IFIH1 | interferon induced with helicase C domain 1 |
| | IDDM19; MGC133047 | | |
| 2.378 | P113; ISGF-3; STAT113; | STAT2 | signal transducer and activator of |
| | MGC59816 | | transcription 2, 113 kDa |
| 2.321 | TL2; APO2L; CD253; TRAIL; | TNFSF10 | tumor necrosis factor (ligand) superfamily, |
| | Apo-2L | | member 10 |
| 2.32 | TEL2; TELB; TEL-2 | ETV7 | ets variant gene 7 (TEL2 oncogene) |
| 2.214 | OIAS; IFI-4; OIASI | OAS1 | 2′,5′-oligoadenylate synthetase 1, 40/46 kDa |
| 2.206 | APT2; PSF2; ABC18; ABCB3; | TAP2 | transporter 2, ATP-binding cassette, sub- |
| | RING11; D6S217E | | family B (MDR/TAP) |
| 2.134 | MGC78578 | OAS2 | 2′-5′-oligoadenylate synthetase 2, 69/71 kDa |
| 2 | VRK2 | VRK2 | vaccinia related kinase 2 |
| 1.975 | PN-I; PSN1; UMPH; UMPH1; | NT5C3 | 5′-nucleotidase, cytosolic III |
| | P5′N-1; cN-III; MGC27337; | | |
| | MGC87109; MGC87828 | | |
| 1.895 | RNF88; TRIM5alpha | TRIM5 | tripartite motif-containing 5 |
| 1.89 | CGI-34; PNAS-2; C9orf83; | CHMP5 | chromatin modifying protein 5 |
| | HSPC177; SNF7DC2 | | |
| 1.863 | ZC3H1; PARP-12; ZC3HDC1; | PARP12 | poly (ADP-ribose) polymerase family, |
| | FLJ22693 | | member 12 |
| 1.845 | PKR; PRKR; EIF2AK1; | EIF2AK2 | eukaryotic translation initiation factor 2- |
| | MGC126524 | | alpha kinase 2 |
| 1.842 | 90K; MAC-2-BP | LGALS3BP | lectin, galactoside-binding, soluble, 3 |
| | | | binding protein |
| 1.807 | RNF88; TRIM5alpha | TRIM5 | tripartite motif-containing 5 |
| 1.743 | C15; onzin | PLAC8 | placenta-specific 8 |
| 1.732 | p48; IRF9; IRF-9; ISGF3 | ISGF3G | interferon-stimulated transcription factor 3, |
| | | | gamma 48 kDa |
| 1.713 | CD317 | BST2 | bone marrow stromal cell antigen 2 |
| 1.665 | ESNA1; ERAP140; FLJ45605; | NCOA7 | nuclear receptor coactivator 7 |
| | MGC88425; Nbla00052; | | |
| | Nbla10993; dJ187J11.3 | | |
| 1.649 | FLJ39275; MGC131926 | ZNFX1 | zinc finger, NFX1-type containing 1 |
| 1.628 | VODI; IFI41; IFI75; FLJ22835 | SP110 | SP110 nuclear body protein |
| 1.627 | EFP; Z147; RNF147; ZNF147 | TRIM25 | tripartite motif-containing 25 |
| 1.523 | NMI | NMI | N-myc (and STAT) interactor |
| 1.505 | TRAP; KIAA1529; | TDRD7 | tudor domain containing 7 |
| | PCTAIRE2BP; RP11- | | |
| | 508D10.1 | | |
| 1.499 | DSH; G1P1; IFI4; p136; | ADAR | adenosine deaminase, RNA-specific |
| | ADAR1; DRADA; DSRAD; | | |
| | IFI-4; K88dsRBP | | |
| 1.494 | C1GALT; T-synthase | C1GALT1 | core 1 synthase, glycoprotein-N- |
| | | | acetylgalactosamine 3-beta- |
| | | | galactosyltransferase, 1 |
| 1.478 | | PHF11 | |
| 1.461 | SCOTIN | SCOTIN | scotin |
| 1.433 | FLJ00340; FLJ34579; | SP100 | SP100 nuclear antigen |
| | DKFZp686E07254 | | |
| 1.415 | FLJ45064 | AGRN | agrin |
| 1.351 | NFTC; OEF1; OEF2; C7orf5; | SAMD9 | sterile alpha motif domain containing 9 |
| | FLJ20073; KIAA2004 | | |
| 1.26 | MEL; RAB8 | RAB8A | RAB8A, member RAS oncogene family |
| 1.215 | 6-16; G1P3; FAM14C; IFI616; | G1P3 | interferon, alpha-inducible protein 6 |
| | IFI-6-16 | | |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. WHO. (World Health Organization, Geneva, 2008).
2. Anderson, S. R., Maguire, H. & Carless, J. Tuberculosis in London: a decade and a half of no decline [corrected]. Thorax 62, 162-7 (2007).
3. Trunz, B. B., Fine, P. & Dye, C. Effect of BCG vaccination on childhood tuberculous meningitis and miliary tuberculosis worldwide: a meta-analysis and assessment of cost-effectiveness. Lancet 367, 1173-80 (2006).
4. Young, D. B., Perkins, M. D., Duncan, K. & Barry, C. E., 3rd. Confronting the scientific obstacles to global control of tuberculosis. J Clin Invest 118, 1255-65 (2008).
5. Center for Communicable Disease Control and Prevention. (ed. U.S. Department of Health and Human Services, C.) XX (Atlanta, Ga., 2007).
6. Pfyffer, G. E., Cieslak, C., Welscher, H. M., Kissling, P. & Rusch-Gerdes, S. Rapid detection of mycobacteria in clinical specimens by using the automated BACTEC 9000 MB system and comparison with radiometric and solid-culture systems. J Clin Microbiol 35, 2229-34 (1997).
7. Schoch, O. D. et al. Diagnostic yield of sputum, induced sputum, and bronchoscopy after radiologic tuberculosis screening. Am J Respir Crit Care Med 175, 80-6 (2007).
8. Storla, D. G., Yimer, S. & Bjune, G. A. A systematic review of delay in the diagnosis and treatment of tuberculosis. BMC Public Health 8, 15 (2008).
9. Comstock, G. W., Livesay, V. T. & Woolpert, S. F. The prognosis of a positive tuberculin reaction in childhood and adolescence. Am J Epidemiol 99, 131-8 (1974).
10. Vynnycky, E. & Fine, P. E. Lifetime risks, incubation period, and serial interval of tuberculosis. Am J Epidemiol 152, 247-63 (2000).
11. Young, D. B., Gideon, H. P. & Wilkinson, R. J. Eliminating latent tuberculosis. Trends Microbiol 17, 183-8 (2009).
12. National Institute for Health and Clinical Excellence. (Royal College of Physicians, UK, 2006).
13. Ottenhoff, T. H. Overcoming the global crisis: “yes, we can”, but also for TB . . . ? Eur J Immunol 39, 2014-20 (2009).
14. Casanova, J. L. & Abel, L. Genetic dissection of immunity to mycobacteria: the human model. Annu Rev Immunol 20, 581-620 (2002).
15. Cooper, A. M. Cell-mediated immune responses in tuberculosis. Annu Rev Immunol 27, 393-422 (2009).
16. Flynn, J. L. & Chan, J. Immunology of tuberculosis. Annu Rev Immunol 19, 93-129 (2001).
17. Keane, J. et al. Tuberculosis associated with infliximab, a tumor necrosis factor alpha-neutralizing agent. N Engl J Med 345, 1098-104 (2001).
18. Chaussabel, D. et al. A modular analysis framework for blood genomics studies: application to systemic lupus erythematosus. Immunity 29, 150-64 (2008).
19. Pascual, V. et al. How the study of children with rheumatic diseases identified interferon-alpha and interleukin-1 as novel therapeutic targets. Immunol Rev 223, 39-59 (2008).
20. Benoist, C., Germain, R. N. & Mathis, D. A plaidoyer for ‘systems immunology’. Immunol Rev 210, 229-34 (2006).
21. Allmark, P. Should research samples reflect the diversity of the population? J Med Ethics 30, 185-9 (2004).
22. Cottin, V. et al. Small-cell lung cancer: patients included in clinical trials are not representative of the patient population as a whole. Ann Oncol 10, 809-15 (1999).
23. Simon, R., Radmacher, M. D., Dobbin, K. & McShane, L. M. Pitfalls in the use of DNA microarray data for diagnostic and prognostic classification. J Natl Cancer Inst 95, 14-8 (2003).
24. Barry, C. E., 3rd et al. The spectrum of latent tuberculosis: rethinking the biology and intervention strategies. Nat Rev Microbiol 7, 845-55 (2009).
25. Center for Communicable Disease Control and Prevention. Misdiagnosis of tuberculosis resulting from laboratory cross-contamination of Mycobacterium tuberculosis cultures. MMWR, New Jersey 49, 413-16 (2000).
26. Pankla, R. et al. Genomic Transcriptional Profiling Identifies a Candidate Blood Biomarker Signature for the Diagnosis of Septicemic Melioidosis. Genome Biol Re-submitted (2009).
27. Beck, J. S., Potts, R. C., Kardjito, T. & Grange, J. M. T4 lymphopenia in patients with active pulmonary tuberculosis. Clin Exp Immunol 60, 49-54 (1985).
28. Rodrigues, D. S. et al Immunophenotypic characterization of peripheral T lymphocytes in Mycobacterium tuberculosis infection and disease. Clin Exp Immunol 128, 149-54 (2002).
29. Auffray, C., Sieweke, M. H. & Geissmann, F. Blood monocytes: development, heterogeneity, and relationship with dendritic cells. Annu Rev Immunol 27, 669-92 (2009).
30. Sher, A. & Coffman, R. L. Regulation of immunity to parasites by T cells and T cell-derived cytokines. Annu Rev Immunol 10, 385-409 (1992).
31. Theofilopoulos, A. N., Baccala, R., Beutler, B. & Kono, D. H. Type I interferons (alpha/beta) in immunity and autoimmunity Annu Rev Immunol 23, 307-36 (2005).
32. Auerbuch, V., Brockstedt, D. G., Meyer-Morse, N., O'Riordan, M. & Portnoy, D. A. Mice lacking the type I interferon receptor are resistant to Listeria monocytogenes. J Exp Med 200, 527-33 (2004).
33. Carrero, J. A., Calderon, B. & Unanue, E. R. Type I interferon sensitizes lymphocytes to apoptosis and reduces resistance to Listeria infection. J Exp Med 200, 535-40 (2004).
34. O'Connell, R. M. et al. Type I interferon production enhances susceptibility to Listeria monocytogenes infection. J Exp Med 200, 437-45 (2004).
35. Bouchonnet, F., Boechat, N., Bonay, M. & Hance, A. J. Alpha/beta interferon impairs the ability of human macrophages to control growth of Mycobacterium bovis BCG. Infect Immun 70, 3020-5 (2002).
36. Manca, C. et al. Hypervirulent M. tuberculosis W/Beijing strains upregulate type I IFNs and increase expression of negative regulators of the Jak-Stat pathway. J Interferon Cytokine Res 25, 694-701 (2005).
37. Stanley, S. A., Johndrow, J. E., Manzanillo, P. & Cox, J. S. The Type I IFN response to infection with Mycobacterium tuberculosis requires ESX-1-mediated secretion and contributes to pathogenesis. J Immunol 178, 3143-52 (2007).
38. Cooper, A. M., Pearl, J. E., Brooks, J. V., Ehlers, S. & Orme, I. M. Expression of the nitric oxide synthase 2 gene is not essential for early control of Mycobacterium tuberculosis in the murine lung. Infect Immun 68, 6879-82 (2000).
39. Shi, S. et al. Expression of many immunologically important genes in Mycobacterium tuberculosis-infected macrophages is independent of both TLR2 and TLR4 but dependent on IFN-alphabeta receptor and STAT1. J Immunol 175, 3318-28 (2005).
40. Farah, R. & Awad, J. The association of interferon with the development of pulmonary tuberculosis. Int J Clin Pharmacol Ther 45, 598-600 (2007).
41. Telesca, C. et al. Interferon-alpha treatment of hepatitis D induces tuberculosis exacerbation in an immigrant. J Infect 54, e223-6 (2007).
42. Eum, S. Y. et al. Neutrophils are the predominant infected phagocytic cells in the airways of patients with active pulmonary tuberculosis. Chest (2009).
43. Eruslanov, E. B. et al. Neutrophil responses to Mycobacterium tuberculosis infection in genetically susceptible and resistant mice. Infect Immun 73, 1744-53 (2005).
44. Barber, D. L. et al. Restoring function in exhausted CD8 T cells during chronic viral infection. Nature 439, 682-7 (2006).
45. Day, C. L. et al. PD-1 expression on HIV-specific T cells is associated with T-cell exhaustion and disease progression. Nature 443, 350-4 (2006).
46. Jurado, J. O. et al. Programmed death (PD)-1:PD-ligand 1/PD-ligand 2 pathway inhibits T cell effector functions during human tuberculosis. J Immunol 181, 116-25 (2008).
47. Boasso, A. et al. PDL-1 upregulation on monocytes and T cells by HIV via type I interferon: restricted expression of type I interferon receptor by CCR5-expressing leukocytes. Clin Immunol 129, 132-44 (2008).
48. Einarsdottir, T., Lockhart, E. & Flynn, J. L. Cytotoxicity and secretion of gamma interferon are carried out by distinct CD8 T cells during Mycobacterium tuberculosis infection. Infect Immun 77, 4621-30 (2009).
49. Ha, S. J., West, E. E., Araki, K., Smith, K. A. & Ahmed, R. Manipulating both the inhibitory and stimulatory immune system towards the success of therapeutic vaccination against chronic viral infections. Immunol Rev 223, 317-33 (2008).
50. Jacobsen, M. et al. Candidate biomarkers for discrimination between infection and disease caused by Mycobacterium tuberculosis. J Mol Med 85, 613-21 (2007).
51. Mistry, R. et al. Gene-expression patterns in whole blood identify subjects at risk for recurrent tuberculosis. J Infect Dis 195, 357-65 (2007).

1-25. (canceled)

26. A method for distinguishing between active and latent Mycobacterium tuberculosis (TB) infection in a patient suspected of being infected with TB, the method comprising:
obtaining a gene expression dataset from a sample from the patient;
determining the expression pattern of four or more genes selected from the genes listed in Table 2 that distinguish between active and latent infection; and
distinguishing between active and latent TB infection based on the expression pattern of the four or more genes.

27. The method of claim 26, wherein the patient gene expression dataset is obtained from cells obtained from at least one of whole blood, peripheral blood mononuclear cells, or sputum.

28. The method of claim 27, wherein the sample is a whole blood sample.

29. The method of claim 26, comprising determining the expression pattern of 5, 10, 15, 20, 40, 50, 70, 80, 90, 100, 125, 150, 200, 250, 300, 350 or 393 genes selected from the genes listed in Table 2 that distinguish between active and latent infection.

30. The method of claim 26, wherein the four or more genes are selected from NAIP, AGMAT, PRDM1, LOC730092, FAM102A, KRT72, KIAA0748, MORC2, CD151, CR1, SPOCK2, SOCS3, DHRS9, P2RY14, BCAS4, MGC22014, RHBDF2, SOCS1, KIAA1026, TLR2, LBH, TPM2, TPD52, FCRLA, NAT6, CLUAP1, PASK, ATP6V0E2, POLR1E, MGC42367, HNRPA1L-2, ALDH1A1, ID3, ZNF429, SNORD13, CD38, C16orf30, CXCL6, SLC30A1, TNFRSF25, ASGR2, MAGEE1, LOC642606, KIAA1641, LOC650795, BMX, CXCL10, EVI2A, LIN7A, ETV7, CLEC12A, TXNDC3, NDRG2, CECR6, TIMM10, MYC, SOD2, ISG15, TXNDC12, IF144L, FER1L3, IFIT5, ZAK, ATP8B2, XAF1, GAS6, PIK31P1, SIPA1L2, H1ST2H2BF, ABLIM1, IKZF3, FAM26F, CAPN12, QPCT, GADD45G, TMEM51, TSHZ2, CD3D, B3GNT8, NR3C2, HERC5, TRIMS, LOC642161, IF16, TCN2, C11orf1, IGF2BP3, LOC728014, LOC648984, DHRS12, ADAM7, BIN1, XRN1, DKFZp761E198, LIMK2, LOC653867, IRF7, KLF12, DKFZp761P0423, PVRIG, CLYBL, ENTPD1, RSAD2, PARP10, CD27, ABHD14A, SATB1, FCGBP, PARP9, C9orf66, EPB41L3, CMPK2, LOC648099, C11orf82, CASP5, FCAR, SRPK1, L00552891, GPR141, PML, CREB5, EPHA4, LOC652755, ZBP1, URG4, CACNA1I, FRMD3, C4 orf18, GPR84, ZNF525, C12orf57, C9orf72, GRAP, NELL2, BLK, AGPAT3, AFF1, IFI44, SP140, TRIB2, GNG10, LOC650799, DDX60, ECGF1, DOCK9, SUCNR1, GZMK, KIAA1618, SIGLECP16, WDFY3, DYSF, ASPHD2, MGC52498, CTSL1, PIK3C2B, SIRPG, ZDHHC19, EPSTI1, STOM, RAB20, SAMD9L, PARP14, LAP3, TRAFD1, SCO2, DTX3L, SESN1, C11orf75, MAPK14, RTP4, LRFN3, FFAR2, CENTA2, UBE2L6, PRRG4, CASP4, MAFB, APOL1, GK, CHMP5, TIFA, STAT1, SESTD1, STAT2, SEPT4, LACTB, DRAM, LOC728744, PSTPIP2, FBXO6, LOC400759, LHFPL2, GADD45B, LOC440731, SQRDL, ACOT9, ANKRD22, C16orf7, DYNLT1, BATF2, PHTF1, WDFY1, SRBD1, SORT1, FLVCR2, BRSK1, and MTHFD2.

31. The method of claim 26, wherein the four or more genes are selected from LOC653610, LILRB4, DUSP3, SLC22A4, SMARCD3, CACNA1E, LILRA5, H1ST2H2AA3, H1ST2H2AC, PLSCR1, SLC26A8, CEACAM1, INCA, COP1, LPCAT2, PFKFB3, ZNF438, KREMEN1, KCNJ15, GYG1, KIF1B, C19orf59, ADM, CDK5RAP2, MEF2D, LOC728519, FCGR1B, LMNB1, GPR109A, ANXA3, and GPR109B.

32. The method of claim 26, further comprising generating a prognosis using the expression pattern of the four or more genes.

33. The method of claim 32, wherein the prognosis comprises identifying a latent TB infection that is likely to become an active TB infection.

34. The method of claim 26, further comprising generating a treatment plan using the expression pattern of the four or more genes.

35. The method of claim 26, further comprising determining whether a treatment has been effective, the method further comprising:
obtaining a second gene expression dataset from a second sample from the patient; and
determining a second expression pattern of the four or more genes;
wherein the treatment has been effective if the second gene expression dataset does not identify an active TB infection.

36. The method of claim 35, comprising determining the expression pattern of 5, 10, 15, 20, 40, 50, 70, 80, 90, 100, 125, 150, 200, 250, 300, 350 or 393 genes selected from the genes listed in Table 2 that distinguish between active and latent infection.

37. The method of claim 35, wherein the four or more genes are selected from NAIP, AGMAT, PRDM1, LOC730092, FAM102A, KRT72, KIAA0748, MORC2, CD151, CR1, SPOCK2, SOCS3, DHRS9, P2RY14, BCAS4, MGC22014, RHBDF2, SOCS1, KIAA1026, TLR2, LBH, TPM2, TPD52, FCRLA, NAT6, CLUAP1, PASK, ATP6V0E2, POLR1E, MGC42367, HNRPA1L-2, ALDH1A1, ID3, ZNF429, SNORD13, CD38, C16orf30, CXCL6, SLC30A1, TNFRSF25, ASGR2, MAGEE1, LOC642606, KIAA1641, LOC650795, BMX, CXCL10, EVI2A, LIN7A, ETV7, CLEC12A, TXNDC3, NDRG2, CECR6, TIMM10, MYC, SOD2, ISG15, TXNDC12, IF144L, FER1L3, IFIT5, ZAK, ATP8B2, XAF1, GAS6, PIK31P1, SIPA1L2, HIST2H2BF, ABLIM1, IKZF3, FAM26F, CAPN12, QPCT, GADD45G, TMEM51, TSHZ2, CD3D, B3GNT8, NR3C2, HERC5, TRIMS, LOC642161, IF16, TCN2, C11orf1, IGF2BP3, LOC728014, LOC648984, DHRS12, ADAM7, BIN1, XRN1, DKFZp761E198, LIMK2, LOC653867, IRF7, KLF12, DKFZp761P0423, PVRIG, CLYBL, ENTPD1, RSAD2, PARP10, CD27, ABHD14A, SATB1, FCGBP, PARP9, C9orf66, EPB41L3, CMPK2, LOC648099, C11orf82, CASP5, FCAR, SRPK1, L00552891, GPR141, PML, CREB5, EPHA4, LOC652755, ZBP1, URG4, CACNA1I, FRMD3, C4 orf18, GPR84, ZNF525, C12orf57, C9orf72, GRAP, NELL2, BLK, AGPAT3, AFF1, IF144, SP140, TRIB2, GNG10, LOC650799, DDX60, ECGF1, DOCK9, SUCNR1, GZMK, KIAA1618, SIGLECP16, WDFY3, DYSF, ASPHD2, MGC52498, CTSL1, PIK3C2B, SIRPG, ZDHHC19, EPSTI1, STOM, RAB20, SAMD9L, PARP14, LAP3, TRAFD1, SCO2, DTX3L, SESN1, C11orf75, MAPK14, RTP4, LRFN3, FFAR2, CENTA2, UBE2L6, PRRG4, CASP4, MAFB, APOL1, GK, CHMP5, TIFA, STAT1, SESTD1, STAT2, SEPT4, LACTB, DRAM, LOC728744, PSTPIP2, FBXO6, LOC400759, LHFPL2, GADD45B, LOC440731, SQRDL, ACOT9, ANKRD22, C16orf7, DYNLT1, BATF2, PHTF1, WDFY1, SRBD1, SORT1, FLVCR2, BRSK1, and MTHFD2.

38. The method of claim 26, wherein the four or more genes are selected from LOC653610, LILRB4, DUSP3, SLC22A4, SMARCD3, CACNA1E, LILRA5, HIST2H2AA3, HIST2H2AC, PLSCR1, SLC26A8, CEACAM1, INCA, COP1, LPCAT2, PFKFB3, ZNF438, KREMEN1, KCNJ15, GYG1, KIF1B, C19orf59, ADM, CDK5RAP2, MEF2D, LOC728519, FCGR1B, LMNB1, GPR109A, ANXA3, and GPR109B.

39. The method of claim 35, wherein the four or more genes are selected from PGLYRP1, IL15, GBP5, and GBP2.

40. The method of claim 35, wherein the four or more genes are selected from CD40LG, OASL, ETS1, HLA-DPB1, CLEC4D, OAS2, DDX58, C5, SERPINA1, CD274, OAS3, IL18RAP, TCF7, OAS1, CD59, BCL6, CCR6, TNFSF13B, FAIM3, CD5, IL7R, EBI2, IFIT3, NBN, IL1RN, TNFAIP6, SERPING1, IFIH1, CD28, GBP6, IF116, IFIT2, TNFSF10, PSME1, TAP2, TRIM22, GBP1, TAP1, PSME2, APOL2, and PSMB9.

41. The method of claim 35, wherein the four or more genes are selected from ABCG1, HK2, MMP9, SOX8, NOV, CDK5R1, SELM, GCH1, ACTA2, SIGLEC5, ATF3, PLAUR, WARS, JAK2, GBP4, STX11, VAMPS, and APOL6.

42. The method of claim 35, wherein the four or more genes are selected from C1QB, OSM, TLR5, IFI35, HPSE, AIM2, FCER1G, LTB4R, IFITM1, FCGR1A, and IFITM3.
